(19) United States
(12) Patent Application Publication
(10) Pub. No.: US 2015/0141714 A1
(43) Pub. Date: May 21, 2015

(54) ENGINEERING PLANTS WITH RATE LIMITING FARNESENE METABOLIC GENES

(71) Applicants: CHROMATIN, INC., Chicago, IL (US); THE OHIO STATE UNIVERSITY, Columbus, OH (US)

(72) Inventors: Joshua Blakeslee, Wooster, OH (US); Katrina Cornish, Wooster, OH (US); Oswald Crasta, Carmel, IN (US); Otto Folkerts, Urbana, IL (US); Dave Jessen, Chanhassen, MN (US); Ramesh Nair, Naperville, IL (US)

(21) Appl. No.: 14/371,765

(22) PCT Filed: Jan. 14, 2013
(86) PCT No.: PCT/US2013/021501

§ 371 Date: Jul. 11, 2014

Related U.S. Application Data
(60) Provisional application No. 61/586,632, filed on Jan. 13, 2012.

(51) Int. Cl. C12P 5/00; C07C 11/21

(52) U.S. Cl. CPC C12P 5/007; C07C 11/21; C12Y 101/01088; C12Y 202/01007; C12Y 205/0101; C12Y 402/03047
USPC 585/16; 435/419; 435/190; 435/193; 435/232; 435/257.2; 435/412; 435/411; 435/415; 435/167

(57) ABSTRACT

The disclosed invention provides methods and compositions for increasing terpenoid production, such as sesquiterpenoids, such as farnesene, in plant cells.

### CROSS REFERENCE TO RELATED APPLICATION

This application claims priority to Blakeslee, J. et al., U.S. Provisional Application No. 61/586,632, “ENGINEERING PLANTS WITH RATE-LIMITING FARNESENE METABOLIC GENES,” filed Jan. 13, 2012, and which is incorporated by reference herein in its entirety.

### GOVERNMENT SUPPORT

The subject matter of this application was in part funded by the Department of Energy, the Advanced Research Projects Agency-Energy under the award “Plant Based Sesquiterpene Biofuels,” DE-AR0000208. The government may have certain rights in this invention.

### FIELD OF THE INVENTION

The present invention relates to engineering plants to express higher levels than endogenous amounts of terpenoids, such as farnesene.

### COMPACT DISC FOR SEQUENCE LISTINGS AND TABLES

Not applicable.

### BACKGROUND OF THE INVENTION

### All Citations are Incorporated Herein by Reference

Agricultural and aquacultural crops have the potential to meet escalating global demands for affordable and sustainable production of food, fuels, fibers, therapeutics, and biofeedstocks.

Development of sustainable sources of domestic energy is crucial for the US to achieve energy independence. In 2010, the US produced 13.2 billion gallons of ethanol from corn grain and 315 million gallons of biodiesel from soybeans as the predominant forms of liquid biofuels (Board, 2011; RFA, 2011). It is expected that biofuels based on corn grain and soybeans will not exceed 15.8 billion gallons in the long term. Although efforts to convert biomass to biofuel by either enzymatic or thermochemical processes will continue to contribute towards energy independence (Lin and Tanaka, 2006; Nigam and Singh, 2011), this process alone is not enough to achieve the target goals of biofuel production. It is projected that only 12% of all liquid fuels produced in the US can be derived from renewable sources by 2035, far below the mandated 30%(Newell, 2011). To reach the target levels of 30% of all liquid fuels consumed in US by 2035, new and innovative biofuel production methodologies must be employed. The research proposed here achieves this goal by producing plants that accumulate μ-farnesene-rich terpene resins that can be converted to liquid fuels. Such crops will yield liquid fuel requiring little external processing, and will keep the US on the cutting-edge of biofuels technology (Connor and Atsumi, 2010).

The terpenoid biosynthetic pathway is ubiquitous in plants and produces over 40,000 structures, forming the largest class of plant metabolites (Bohlmann and Keeling, 2008). To date, research on terpenoids has focused primarily on uses as flavor components or scent compounds (Cheng et al., 2007). Because of their abundance and high energy content terpenoids provide an attractive alternative to current biofuels (Bohlmann and Keeling, 2008; Pourbafrani et al., 2010; Wu et al., 2006). To date, terpene based biofuel production has focused on the use of micro-organisms, including yeast and bacterial systems, to generate poly-terpenoid fuels (Fischer et al., 2008; Nigam and Singh, 2011; Peralta-Yahya and Keasling, 2010). However, it is unclear whether this microorganism-based approach will allow production of isoprenoid resins at sufficient quantities to supplement and/or replace liquid fossil fuel consumption. Further, this process is energy-intensive, requiring a supply of plant-based sugars for large scale fermentation, constant maintenance of temperature and nutrition to micro-organism cultures, and the development of immense infrastructure to support meaningful, large-scale micro-organism growth. Attempts have been made to overcome these obstacles by engineering the production of biodiesel hydrocarbons in algal systems and thus defray some of the energy cost by harnessing the photosynthetic capacity of these organisms. Algal systems still require significant inputs of energy to maintain temperature and salt equilibria, and have failed to produce biodiesel in sufficient quantities to offset the costs of building the large-scale bio-reactors necessary for algal biodiesel production.

Guayule, a dicotyledonous desert shrub native to the Southwestern US and Mexico thrives in semi-arid desert environments and marginal lands not currently used for food production (Bonner, 1943; Hammond, 1965; Tipton and Gregg, 1982). Guayule has long been established as a source of natural rubber, resins, and bioactive terpenoid compounds. In addition to producing hydrocarbon rubber polymers during the winter (Cornish and Backhaus, 2003), guayule produces and stores a high-energy hydrocarbon terpenoid resin in specialized resin vessels throughout the year (Coffelt et al., 2009b). Further, guayule can be grown with greatly reduced inputs of water (Dierig et al., 2001) and pesticides (compared to traditional crops such as nuts, alfalfa, and cotton), and on lands in the Southwestern US not currently utilized for food production (Whitworth, 1991).

Guayule has been successfully transformed to express several genes involved in the synthesis of terpenoid precursors; mono-, sesqui- and di-terpenoid molecules; and isoprenoid rubber polymers using Agrobacterium-mediated transformation (Veatch et al., 2005). Further, methods have been developed for the optimal extraction of resin and terpenoid moieties from harvested guayule tissues (Pearson et al., 2010; Salvucci et al., 2009). Finally, transgenic guayule lines have been successfully brought to field trials, where they have been demonstrated to accumulate increased accumulations of terpenoid-rich resins (Veatch et al., 2005).

Recent plant breeding efforts to improve guayule have resulted in the development of twenty publically-available improved guayule lines (with maximum yield of 830-1000 lb/rubber/acre/year)(Dierig, 1996; Estilai, 1985; Estilai, 1986; Estilai, 1994; Niehaus, 1983; Ray et al., 1999; Tysdal et al., 1983) with 7-15% resin.

Sorghum, a C4 monocotyledonous grass grown in the southwestern, central and Midwestern US, has high photosynthetic efficiency, water and nutrient efficiency, stress tolerance, and is unmatched in its diversity of germplasm including starch (grain) types, high sugar (sweet) types, and high-biomass photoperiod sensitive (forage) types. Sorghum outperforms corn in regions with low annual rainfall, making it an ideal crop for the semi-arid regions (Zhan et al., 2003). Sorghum is suited to acreage where corn, soybean and cotton are cultivated on an additional 70 million Ha in the US.

### SUMMARY OF THE INVENTION

In a first aspect, the invention is directed to methods of making a plant cell having increased production of at least one terpenoid native to a plant, the method comprising expressing in a plant cell a heterologous nucleic acid encoding for (a) HMG-CoA reductase, (b) 1-deoxy-D-xylulose-5-phosphate synthase, (c) farnesyl pyrophosphate synthase, and (d) β-farnesene synthase, wherein production of the at least one terpenoid is significantly increased when compared to a wild-type plant cell not encoding the heterologous nucleic acids. In further aspects, the HMG-CoA reductase is an Arabidopsis, Oryza, Saccharomyces, or Hevea HMG-CoA reductase; the 1-deoxy-D-xyululose-5-phophate is an Arabidopsis, Oryza, Saccharomyces, or Zea 1-deoxy-D-xyululose; the farnesyl pyrophosphate synthase is an Arabidopsis, Oryza, or Solanum farnesyl pyrophosphate; or the β-farnesene synthase is an Arabidopsis, Oryza, or Artemisia β-farnesene synthase. In yet additional aspects, the HMG-CoA reductase is an Arabidopsis thaliana, Oryza sativa, Saccharomyces cerevisiae, or Hevea HMG-CoA reductase; the 1-deoxy-D-xyululose-5-phophate is an Arabidopsis thaliana, Oryza sativa, Saccharomyces cerevisiae, or Zea mays 1-deoxy-D-xyululose; the farnesyl pyrophosphate synthase is an Arabidopsis thaliana, Oryza sativa, or Solanum lycopersicon farnesyl pyrophosphate; the β-farnesene synthase is an Arabidopsis thaliana, Oryza sativa, or Artemisia annua β-farnesene synthase. In even further aspects, at least one of the heterologous nucleic acids is codon-optimized for expression in a plant. In additional aspects, the HMG-CoA reductase is encoded by a polynucleotide having at least 70% sequence identity to a nucleic acid sequence selected from the group consisting of SEQ ID NOs:1, 2, 3, 16, 17, and 28; the 1-deoxy-D-xyululose-5-phophate is encoded by a polynucleotide having at least 70% sequence identity to a nucleic acid sequence selected from the group consisting of SEQ ID NOs: 4, 5, 6, 18, 19 and 20; the farnesyl pyrophosphate synthase is encoded by a polynucleotide having at least 70% sequence identity to a nucleic acid sequence selected from the group consisting of SEQ ID NOs:7, 8, 9, 21, 22, 23, 24 and 29; the β-farnesene synthase is encoded by a polynucleotide having at least 70% sequence identity to a nucleic acid sequence selected from the group consisting of SEQ ID NOs:10, 11, 12, 25, and 26; and the AVP1/OMP1 may be encoded by a polynucleotide having at least 70% sequence identity to a nucleic acid sequence selected from the group consisting of SEQ ID NOs:13, 14, 15, or 27. In additional aspects, the HMG-CoA reductase is encoded by a polynucleotide having at a nucleic acid sequence of SEQ ID NOs:1, 2, 3, 16, 17, or 28; a 1-deoxy-D-xyululose-5-phophate is encoded by a polynucleotide having at a nucleic acid sequence of SEQ ID NOs:4, 5, 6, 18, 19, or 20; a farnesyl pyrophosphate synthase is encoded by a polynucleotide having at a nucleic acid sequence selected from the group consisting of SEQ ID NOs:7, 8, 9, 21, 22, 23, 24, or 29; optionally; the aspects may further comprise an AVP1/OMP1 is encoded by a polynucleotide having at a nucleic acid sequence selected from the group consisting of SEQ ID NOs:13, 14, 15, or 27; or a β-farnesene synthase is encoded by a polynucleotide having at a nucleic acid sequence selected from the group consisting of SEQ ID NOs:10, 11, 12, 25 or 26. Such methods may further include heterologous polynucleotides that comprise a nucleic acid sequence encoding an FVE or a GWD gene.

In additional aspects, the methods comprise making a plant cell comprising HMG-CoA reductase, farnesyl pyrophosphate synthase, β-farnesene synthase and AVP1/OMP1 heterologous nucleic acids; in further aspects, such heterologous nucleic acids are operably linked to constitutive promoters. In an additional aspect, the methods comprising making a plant cell comprising plant HMG-CoA reductase, farnesyl pyrophosphate synthase, and β-farnesene synthase heterologous nucleic acids; in further aspects, such heterologous nucleic acids are operably linked to tissue or developmental specific promoters, such as lignin-specific promoters. In yet an additional aspect, the methods comprise making a plant cell comprising 1-deoxy-D-xylulose-5-phosphate synthase, farnesyl pyrophosphate synthase and β-farnesene synthase heterologous nucleic acids; in further such aspects, the heterologous nucleic acids target the encoded polypeptides to the chloroplast; in yet further aspects, such heterologous nucleic acids are operably linked to constitutive promoters. In any of these previous aspects, the HMG-CoA reductase may be encoded by a polynucleotide having at least 70% sequence identity to a nucleic acid sequence selected from the group consisting of SEQ ID NOs:1, 2, 3, 16, 17, and 28; the 1-deoxy-D-xyululose-5-phophate may be encoded by a polynucleotide having at least 70% sequence identity to a nucleic acid sequence selected from the group consisting of SEQ ID NOs: 4, 5, 6, 18, 19 and 20; the farnesyl pyrophosphate synthase may be encoded by a polynucleotide having at least 70% sequence identity to a nucleic acid sequence selected from the group consisting of SEQ ID NOs:7, 8, 9, 21, 22, 23, 24 and 29; the β-farnesene synthase may be encoded by a polynucleotide having at least 70% sequence identity to a nucleic acid sequence selected from the group consisting of SEQ ID NOs:10, 11, 12, 25, and 26; and the AVP1/OMP1 may be encoded by a polynucleotide having at least 70% sequence identity to a nucleic acid sequence selected from the group consisting of SEQ ID NOs:13, 14, 15, or 27. In additional aspects, the HMG-CoA reductase may be encoded by a polynucleotide having at a nucleic acid sequence of SEQ ID NOs:1, 2, 3, 16, 17, or 28; a 1-deoxy-D-xyululose-5-phophate may be encoded by a polynucleotide having at a nucleic acid sequence of SEQ ID NOs:4, 5, 6, 18, 19, or 20; a farnesyl pyrophosphate synthase may be encoded by a polynucleotide having at a nucleic acid sequence selected from the group consisting of SEQ ID NOs:7, 8, 9, 21, 22, 23, 24, or 29; an AVP1/OMP1 may be encoded by a polynucleotide having at a nucleic acid sequence selected from the group consisting of SEQ ID NOs:13, 14, 15, or 27; or a β-farnesene synthase may be encoded by a polynucleotide having at a nucleic acid sequence selected from the group consisting of SEQ ID NOs:10, 11, 12, 25 or 26.

In further aspects, the methods of the invention comprise plant cells that are from a plant selected from the group consisting of a green algae, a vegetable crop plant, a fruit crop plant, a vine crop plant, a field crop plant, a biomass plant, a bedding plant, and a tree; such plants may be selected from the group consisting of corn, soybean, Brassica, tomato, sorghum, sugarcane, guayule, miscanthus, switchgrass, wheat, barley, oat, rye, wheat, rice, beet, green algae and cotton.

In yet further aspects, the methods of the invention are directed to making plant cells that are guayule plant cells, and the cells express an HMG-CoA reductase is encoded by a polynucleotide having at least 70% sequence identity to a nucleic acid sequence of SEQ ID NOs:1, 2, 3, 16, 17, or 28; a 1-deoxy-D-xyululose-5-phophate is encoded by a polynucleotide having at least 70% sequence identity to a nucleic acid sequence of SEQ ID NOs:4, 5, 6, 18, 19, or 20; a farnesyl pyrophosphate synthase is encoded by a polynucleotide having at least 70% sequence identity to a nucleic acid sequence selected from the group consisting of SEQ ID NOs:7, 8, 9, 21, 22, 23, 24, or 29; an AVP1/OMP1 is encoded by a polynucleotide having at least 70% sequence identity to a nucleic acid sequence selected from the group consisting of SEQ ID NOs:13, 14, 15, or 27; or a β-farnesene synthase is encoded by a polynucleotide having at least 70% sequence identity to a nucleic acid sequence selected from the group consisting of SEQ ID NOs:10, 11, 12, 25 or 26. In yet further such aspect, the methods comprising making guayule plant cells the further express an HMG-CoA reductase is encoded by a polynucleotide having a nucleic acid sequence of SEQ ID NOs:1, 2, 3, 16, 17, or 28; a 1-deoxy-D-xyululose-5-phophate is encoded by a polynucleotide having a nucleic acid sequence of SEQ ID NOs:4, 5, 6, 18, 19, or 20; a farnesyl pyrophosphate synthase is encoded by a polynucleotide having a nucleic acid sequence selected from the group consisting of SEQ ID NOs:7, 8, 9, 21, 22, 23, 24, or 29; an AVP1/OMP1 is encoded by a polynucleotide having a nucleic acid sequence selected from the group consisting of SEQ ID NOs:13, 14, 15, or 27; or a β-farnesene synthase is encoded by a polynucleotide having a nucleic acid sequence selected from the group consisting of SEQ ID NOs:10, 11, 12, 25 or 26.

In further aspects, the invention is directed to methods of making sorghum plant cells, and the cell expresses an HMG-CoA reductase is encoded by a polynucleotide having at least 70% sequence identity to a nucleic acid sequence of SEQ ID NOs:1, 2, 3, 16, 17, or 28; a 1-deoxy-D-xyululose-5-phophate is encoded by a polynucleotide having at least 70% sequence identity to a nucleic acid sequence of SEQ ID NOs:4, 5, 6, 18, 19, or 20; a farnesyl pyrophosphate synthase is encoded by a polynucleotide having at least 70% sequence identity to a nucleic acid sequence selected from the group consisting of SEQ ID NOs:7, 8, 9, 21, 22, 23, 24, or 29; an AVP1/OMP1 is encoded by a polynucleotide having at least 70% sequence identity to a nucleic acid sequence selected from the group consisting of SEQ ID NOs:13, 14, 15, or 27; or a β-farnesene synthase is encoded by a polynucleotide having at least 70% sequence identity to a nucleic acid sequence selected from the group consisting of SEQ ID NOs:10, 11, 12, 25 or 26. In further such aspects, the methods of making sorghum plant cells express an HMG-CoA reductase is encoded by a polynucleotide having a nucleic acid sequence of SEQ ID NOs:1, 2, 3, 16, 17, or 28; a 1-deoxy-D-xyululose-5-phophate is encoded by a polynucleotide having a nucleic acid sequence of SEQ ID NOs:4, 5, 6, 18, 19, or 20; a farnesyl pyrophosphate synthase is encoded by a polynucleotide having a nucleic acid sequence selected from the group consisting of SEQ ID NOs:7, 8, 9, 21, 22, 23, 24, or 29; an AVP1/OMP1 is encoded by a polynucleotide having a nucleic acid sequence selected from the group consisting of SEQ ID NOs:13, 14, 15, or 27; or a β-farnesene synthase is encoded by a polynucleotide having a nucleic acid sequence selected from the group consisting of SEQ ID NOs:10, 11, 12, 25 or 26.

In further aspects, the invention is directed to methods of making sugarcane plant cells, and the cell expresses an HMG-CoA reductase is encoded by a polynucleotide having at least 70% sequence identity to a nucleic acid sequence of SEQ ID NOs:1, 2, 3, 16, 17, or 28; a 1-deoxy-D-xyululose-5-phophate is encoded by a polynucleotide having at least 70% sequence identity to a nucleic acid sequence of SEQ ID NOs:4, 5, 6, 18, 19, or 20; a farnesyl pyrophosphate synthase is encoded by a polynucleotide having at least 70% sequence identity to a nucleic acid sequence selected from the group consisting of SEQ ID NOs:7, 8, 9, 21, 22, 23, 24, or 29; an AVP1/OMP1 is encoded by a polynucleotide having at least 70% sequence identity to a nucleic acid sequence selected from the group consisting of SEQ ID NOs:13, 14, 15, or 27; or a β-farnesene synthase is encoded by a polynucleotide having at least 70% sequence identity to a nucleic acid sequence selected from the group consisting of SEQ ID NOs:10, 11, 12, 25 or 26. In further such aspects, the methods of making sugarcane plant cells express an HMG-CoA reductase is encoded by a polynucleotide having a nucleic acid sequence of SEQ ID NOs:1, 2, 3, 16, 17, or 28; a 1-deoxy-D-xyululose-5-phophate is encoded by a polynucleotide having a nucleic acid sequence of SEQ ID NOs:4, 5, 6, 18, 19, or 20; a farnesyl pyrophosphate synthase is encoded by a polynucleotide having a nucleic acid sequence selected from the group consisting of SEQ ID NOs:7, 8, 9, 21, 22, 23, 24, or 29; an AVP1/OMP1 is encoded by a polynucleotide having a nucleic acid sequence selected from the group consisting of SEQ ID NOs:13, 14, 15, or 27; or a β-farnesene synthase is encoded by a polynucleotide having a nucleic acid sequence selected from the group consisting of SEQ ID NOs:10, 11, 12, 25 or 26.

In all previous aspects, the at least one terpenoid is a sesquiterpenoid, wherein the sesquiterpenoid is farnesene.

In the above aspects, the methods may further comprise theat least one heterologous nucleic acid is operably linked to a constitutive promoter or to an inducible or tissue-specific promoter.

In the above aspects, the methods may further comprise making the plant cells comprising an autonomous DNA construct in the plant cell that comprises at least one heterologous nucleic acid. Such autonomous DNA constructs may be mini-chromosomes, and wherein such mini-chromosomes may comprise a centromere derived from the species of the plant cell.

In the above aspects, the methods may further comprise isolating the farnesene; such isolated farnesene may further be processed into farnesene.

In a second aspect, the invention is directed to a plant cell having increased production of at least one terpenoid native to a plant, the method comprising expressing in a plant cell a heterologous nucleic acid encoding for (a) HMG-CoA reductase, (b) 1-deoxy-D-xylulose-5-phosphate synthase, (c) farnesyl pyrophosphate synthase, and (d) β-farnesene synthase, wherein production of the at least one terpenoid is significantly increased when compared to a wild-type plant cell not encoding the heterologous nucleic acids. In further aspects, the HMG-CoA reductase is an Arabidopsis, Oryza, Saccharomyces, or Hevea HMG-CoA reductase; the 1-deoxy-D-xyululose-5-phophate is an Arabidopsis, Oryza, Saccharomyces, or Zea 1-deoxy-D-xyululose; the farnesyl pyrophosphate synthase is an Arabidopsis, Oryza, or Solanum farnesyl pyrophosphate; or the β-farnesene synthase is an Arabidopsis, Oryza, or Artemisia β-farnesene synthase. In yet additional aspects, the HMG-CoA reductase is an Arabidopsis thaliana, Oryza sativa, Saccharomyces cerevisiae, or Hevea HMG-CoA reductase; the 1-deoxy-D-xyululose-5-phophate is an Arabidopsis thaliana, Oryza sativa, Saccharomyces cerevisiae, or Zea mays 1-deoxy-D-xyululose; the farnesyl pyrophosphate synthase is an Arabidopsis thaliana, Oryza sativa, or Solanum lycopersicon farnesyl pyrophosphate; the β-farnesene synthase is an Arabidopsis thaliana, Oryza sativa, or Artemisia annua β-farnesene synthase. In even further aspects, at least one of the heterologous nucleic acids is codon-optimized for expression in a plant. In additional aspects, the HMG-CoA reductase is encoded by a polynucleotide having at least 70% sequence identity to a nucleic acid sequence selected from the group consisting of SEQ ID NOs:1, 2, 3, 16, 17, and 28; the 1-deoxy-D-xyululose-5-phophate is encoded by a polynucleotide having at least 70% sequence identity to a nucleic acid sequence selected from the group consisting of SEQ ID NOs: 4, 5, 6, 18, 19 and 20; the farnesyl pyrophosphate synthase is encoded by a polynucleotide having at least 70% sequence identity to a nucleic acid sequence selected from the group consisting of SEQ ID NOs:7, 8, 9, 21, 22, 23, 24 and 29; the β-farnesene synthase is encoded by a polynucleotide having at least 70% sequence identity to a nucleic acid sequence selected from the group consisting of SEQ ID NOs:10, 11, 12, 25, and 26; and the AVP1/OMP1 may be encoded by a polynucleotide having at least 70% sequence identity to a nucleic acid sequence selected from the group consisting of SEQ ID NOs:13, 14, 15, or 27. In additional aspects, the HMG-CoA reductase is encoded by a polynucleotide having at a nucleic acid sequence of SEQ ID NOs:1, 2, 3, 16, 17, or 28; a 1-deoxy-D-xyululose-5-phophate is encoded by a polynucleotide having at a nucleic acid sequence of SEQ ID NOs:4, 5, 6, 18, 19, or 20; a farnesyl pyrophosphate synthase is encoded by a polynucleotide having at a nucleic acid sequence selected from the group consisting of SEQ ID NOs:7, 8, 9, 21, 22, 23, 24, or 29; optionally; the aspects may further comprise an AVP1/OMP1 is encoded by a polynucleotide having at a nucleic acid sequence selected from the group consisting of SEQ ID NOs:13, 14, 15, or 27; or a β-farnesene synthase is encoded by a polynucleotide having at a nucleic acid sequence selected from the group consisting of SEQ ID NOs:10, 11, 12, 25 or 26. Such cells may further include heterologous polynucleotides that comprise a nucleic acid sequence encoding an FVE or a GWD gene.

In additional aspects, the invention is directed to a plant cell comprising HMG-CoA reductase, farnesyl pyrophosphate synthase, β-farnesene synthase and AVP1/OMP1 heterologous nucleic acids; in further aspects, such heterologous nucleic acids are operably linked to constitutive promoters. In an additional aspect, the plant cell comprises plant HMG-CoA reductase, farnesyl pyrophosphate synthase, and β-farnesene synthase heterologous nucleic acids; in further aspects, such heterologous nucleic acids are operably linked to tissue or developmental specific promoters, such as lignin-specific promoters. In yet an additional aspect, the plant cell comprises 1-deoxy-D-xylulose-5-phosphate synthase, farnesyl pyrophosphate synthase and β-farnesene synthase heterologous nucleic acids; in further such aspects, the heterologous nucleic acids target the encoded polypeptides to the chloroplast; in yet further aspects, such heterologous nucleic acids are operably linked to constitutive promoters. In any of these previous aspects, the HMG-CoA reductase may be encoded by a polynucleotide having at least 70% sequence identity to a nucleic acid sequence selected from the group consisting of SEQ ID NOs:1, 2, 3, 16, 17, and 28; the 1-deoxy-D-xyululose-5-phophate may be encoded by a polynucleotide having at least 70% sequence identity to a nucleic acid sequence selected from the group consisting of SEQ ID NOs: 4, 5, 6, 18, 19 and 20; the farnesyl pyrophosphate synthase may be encoded by a polynucleotide having at least 70% sequence identity to a nucleic acid sequence selected from the group consisting of SEQ ID NOs:7, 8, 9, 21, 22, 23, 24 and 29; the β-farnesene synthase may be encoded by a polynucleotide having at least 70% sequence identity to a nucleic acid sequence selected from the group consisting of SEQ ID NOs:10, 11, 12, 25, and 26; and the AVP1/OMP1 may be encoded by a polynucleotide having at least 70% sequence identity to a nucleic acid sequence selected from the group consisting of SEQ ID NOs:13, 14, 15, or 27. In additional aspects, the HMG-CoA reductase may be encoded by a polynucleotide having at a nucleic acid sequence of SEQ ID NOs:1, 2, 3, 16, 17, or 28; a 1-deoxy-D-xyululose-5-phophate may be encoded by a polynucleotide having at a nucleic acid sequence of SEQ ID NOs:4, 5, 6, 18, 19, or 20; a farnesyl pyrophosphate synthase may be encoded by a polynucleotide having at a nucleic acid sequence selected from the group consisting of SEQ ID NOs:7, 8, 9, 21, 22, 23, 24, or 29; an AVP1/OMP1 may be encoded by a polynucleotide having at a nucleic acid sequence selected from the group consisting of SEQ ID NOs:13, 14, 15, or 27; or a β-farnesene synthase may be encoded by a polynucleotide having at a nucleic acid sequence selected from the group consisting of SEQ ID NOs:10, 11, 12, 25 or 26.

In further aspects, the methods of the invention comprise plant cells that are from a plant selected from the group consisting of a green algae, a vegetable crop plant, a fruit crop plant, a vine crop plant, a field crop plant, a biomass plant, a bedding plant, and a tree; such plants may be selected from the group consisting of corn, soybean, Brassica, tomato, sorghum, sugarcane, guayule, miscanthus, switchgrass, wheat, barley, oat, rye, wheat, rice, beet, green algae and cotton.

In yet further aspects, the plant cells of the invention are directed to plant cells that are guayule plant cells, and the cells express an HMG-CoA reductase is encoded by a polynucleotide having at least 70% sequence identity to a nucleic acid sequence of SEQ ID NOs:1, 2, 3, 16, 17, or 28; a 1-deoxy-D-xyululose-5-phophate is encoded by a polynucleotide having at least 70% sequence identity to a nucleic acid sequence of SEQ ID NOs:4, 5, 6, 18, 19, or 20; a farnesyl pyrophosphate synthase is encoded by a polynucleotide having at least 70% sequence identity to a nucleic acid sequence selected from the group consisting of SEQ ID NOs:7, 8, 9, 21, 22, 23, 24, or 29; an AVP1/OMP1 is encoded by a polynucleotide having at least 70% sequence identity to a nucleic acid sequence selected from the group consisting of SEQ ID NOs:13, 14, 15, or 27; or a β-farnesene synthase is encoded by a polynucleotide having at least 70% sequence identity to a nucleic acid sequence selected from the group consisting of SEQ ID NOs:10, 11, 12, 25 or 26. In yet further such aspect, the plant cells comprise guayule plant cells that further express an HMG-CoA reductase is encoded by a polynucleotide having a nucleic acid sequence of SEQ ID NOs:1, 2, 3, 16, 17, or 28; a 1-deoxy-D-xyululose-5-phophate is encoded by a polynucleotide having a nucleic acid sequence of SEQ ID NOs:4, 5, 6, 18, 19, or 20; a farnesyl pyrophosphate synthase is encoded by a polynucleotide having a nucleic acid sequence selected from the group consisting of SEQ ID NOs:7, 8, 9, 21, 22, 23, 24, or 29; an AVP1/OMP1 is encoded by a polynucleotide having a nucleic acid sequence selected from the group consisting of SEQ ID NOs:13, 14, 15, or 27; or a β-farnesene synthase is encoded by a polynucleotide having a nucleic acid sequence selected from the group consisting of SEQ ID NOs:10, 11, 12, 25 or 26.

In further aspects, the invention is directed to sorghum plant cells, and the cell expresses an HMG-CoA reductase is encoded by a polynucleotide having at least 70% sequence identity to a nucleic acid sequence of SEQ ID NOs:1, 2, 3, 16, 17, or 28; a 1-deoxy-D-xyululose-5-phophate is encoded by a polynucleotide having at least 70% sequence identity to a nucleic acid sequence of SEQ ID NOs:4, 5, 6, 18, 19, or 20; a farnesyl pyrophosphate synthase is encoded by a polynucleotide having at least 70% sequence identity to a nucleic acid sequence selected from the group consisting of SEQ ID NOs:7, 8, 9, 21, 22, 23, 24, or 29; an AVP1/OMP1 is encoded by a polynucleotide having at least 70% sequence identity to a nucleic acid sequence selected from the group consisting of SEQ ID NOs:13, 14, 15, or 27; or a β-farnesene synthase is encoded by a polynucleotide having at least 70% sequence identity to a nucleic acid sequence selected from the group consisting of SEQ ID NOs:10, 11, 12, 25 or 26. In further such aspects, the sorghum plant cells express an HMG-CoA reductase is encoded by a polynucleotide having a nucleic acid sequence of SEQ ID NOs:1, 2, 3, 16, 17, or 28; a 1-deoxy-D-xyululose-5-phophate is encoded by a polynucleotide having a nucleic acid sequence of SEQ ID NOs:4, 5, 6, 18, 19, or 20; a farnesyl pyrophosphate synthase is encoded by a polynucleotide having a nucleic acid sequence selected from the group consisting of SEQ ID NOs:7, 8, 9, 21, 22, 23, 24, or 29; an AVP1/OMP1 is encoded by a polynucleotide having a nucleic acid sequence selected from the group consisting of SEQ ID NOs:13, 14, 15, or 27; or a β-farnesene synthase is encoded by a polynucleotide having a nucleic acid sequence selected from the group consisting of SEQ ID NOs:10, 11, 12, 25 or 26.

In further aspects, the invention is directed to sugarcane plant cells, and the cell expresses an HMG-CoA reductase is encoded by a polynucleotide having at least 70% sequence identity to a nucleic acid sequence of SEQ ID NOs:1, 2, 3, 16, 17, or 28; a 1-deoxy-D-xyululose-5-phophate is encoded by a polynucleotide having at least 70% sequence identity to a nucleic acid sequence of SEQ ID NOs:4, 5, 6, 18, 19, or 20; a farnesyl pyrophosphate synthase is encoded by a polynucleotide having at least 70% sequence identity to a nucleic acid sequence selected from the group consisting of SEQ ID NOs:7, 8, 9, 21, 22, 23, 24, or 29; an AVP1/OMP1 is encoded by a polynucleotide having at least 70% sequence identity to a nucleic acid sequence selected from the group consisting of SEQ ID NOs:13, 14, 15, or 27; or a β-farnesene synthase is encoded by a polynucleotide having at least 70% sequence identity to a nucleic acid sequence selected from the group consisting of SEQ ID NOs:10, 11, 12, 25 or 26. In further such aspects, the sugarcane plant cells express an HMG-CoA reductase is encoded by a polynucleotide having a nucleic acid sequence of SEQ ID NOs:1, 2, 3, 16, 17, or 28; a 1-deoxy-D-xyululose-5-phophate is encoded by a polynucleotide having a nucleic acid sequence of SEQ ID NOs:4, 5, 6, 18, 19, or 20; a farnesyl pyrophosphate synthase is encoded by a polynucleotide having a nucleic acid sequence selected from the group consisting of SEQ ID NOs:7, 8, 9, 21, 22, 23, 24, or 29; an AVP1/OMP1 is encoded by a polynucleotide having a nucleic acid sequence selected from the group consisting of SEQ ID NOs:13, 14, 15, or 27; or a β-farnesene synthase is encoded by a polynucleotide having a nucleic acid sequence selected from the group consisting of SEQ ID NOs:10, 11, 12, 25 or 26.

In all previous aspects, the at least one terpenoid is a sesquiterpenoid, wherein the sesquiterpenoid is farnesene.

In the above aspects, the plant cells may further comprise the at least one heterologous nucleic acid is operably linked to a constitutive promoter or to an inducible or tissue-specific promoter.

In the above aspects, the plant cells may further comprise an autonomous DNA construct in the plant cell that comprises at least one heterologous nucleic acid. Such autonomous DNA constructs may be mini-chromosomes, and wherein such mini-chromosomes may comprise a centromere derived from the species of the plant cell.

In the above aspects, farnesene may be isolated from the plant cells of the invention; such isolated farnesene may further be processed into farnesene.

The invention is also directed to fuels comprising a terpenoid made according to any of the methods of the invention, or made by a plant cell of the invention. In such fuels, the terpenoid is a sesquiterpenoid, such as farnesene.

### BRIEF DESCRIPTION OF SEVERAL VIEWS OF THE DRAWINGS

FIG. 1 shows a schema of β-farnesene production strategies. Glycolysis breaks sucrose into pyruvate which is processed into the terpenoid precursors DMAPP/IPP via the MVA (cytosol) or MEP (chloroplast) pathway. IPP subunits are assembled into farnesyl-pyrophosphate (FPP), which is then converted into β-farnesene. Proteins catalyzing rate-limiting steps are HMG-CoA reductase, FPP synthase, β-farnesene synthase, and 1-deoxy-D-xylulose-5-phosphate synthase.

FIG. 2 shows GC-eiMS quantitation of AL2 leaf extract (Sc-HMGR, Sc-FPPS, Aa-bFS, Os-VP1; constitutive). Internal standard trichlorobenzene (Rt 4.1 min.) is present at 0.73 micrograms/mL. Unidentified sesquiterpenes present at Rₜ ca. 5.9, 6.2, and 6.5 minutes. Monoterpenes would elute near 4 minutes under these conditions. See Example 7 for further details.

FIG. 3 shows GC trace of AL414 extract (CTP-Os-DXS, CTP-Aa-bFS, CTP-Sc-FPPS; constitutive). Internal standard trichlorobenzene (Rt 4.1 min.) is present at 0.73 micrograms/mL. Trace amounts of sesquiterpenes may be present at Rₜ ca. 5.9 and 6.5 minutes. Monoterpenes would elute near 4 minutes under these conditions. See Example 7 for further details.

### DETAILED DESCRIPTION OF THE INVENTION

### I. Introduction

The present invention provides for plants that accumulate β-farnesene-rich terpene resins that can be converted to liquid fuels. Such crops yield liquid fuel requiring little external processing (Connor and Atsumi, 2010).

The invention represents a departure from current biofuel approaches, as it creates crop systems that can generate liquid terpenoid, such as sesquiterpenoid, resin biofuels in sufficient quantities to meet 30% of annual US energy needs (Newell, 2011). This approach offers several advantages over current biofuel technologies. Unlike starch or cellulose based ethanol production this process does not require harsh pretreatment steps, saccharification and fermentation, thus reducing the expensive infrastructure needed for biofuel production. The fuel itself has unique properties such as immiscibility with water, thus avoiding expensive distillation processes needed to concentrate fuel produced by starch and cellulosic technologies. Compared to current biodiesel production, extraction of β-farnesene from biomass and conversion to farnesane requires a simple extraction process, reducing overall production cost, and conversion of β-farnesene to farnesane is a one-step hydrogenation process. Unlike biodiesel currently produced from soy or canola seed oil, the whole plant can be used, providing opportunities for higher biofuel yields per hectare and reduced competition between food and feed.

The invention takes a unique approach to overcome hurdles encountered in current efforts to generate biofuels from terpenoid and biodiesel production in microorganisms, such as yeasts and algae. In some embodiments, energy inputs are drastically reduced by utilizing the photosynthetic capacity of an entire plant and funneling all non-essential carbon into the production of β-farnesene-enriched resins, such as is possible in plants like guayule or sweet sorghum. These resins can be used as a readily-extractable liquid biofuel. Furthermore production of biofuel in crops do not require the cost associated with developing microbial fermentation processes and facilities and can capitalize on a vast existing agricultural infrastructure.

In some embodiments of the invention, guayule or sweet sorghum is modified to produce large quantities of the terpenoids. Guayule can be grown on approximately 40 million Ha of currently uncultivated marginal land. Drought-tolerant sorghum can be grown on more than 70 million Ha where bioenergy crops are currently farmed. Production of liquid β-farnesene biofuel in these two geographically distinct crops produce low-cost transportation fuel and allow diversification of feedstock supply and land use with minimal impact on food crops. In contrast, 1 Ha of soybeans can produce about 150-250 gallons of biodiesel, while engineered plants containing, for example, 20% by dry weight of farnesene at 39-56 t/Ha of harvested yield have the production potential of 1800-2800 gallons of biofuel/Ha. Further, engineered plants containing 20% farnesene by dry weight when processed, can produce 250-388 GJ/Ha/year of biofuel with an energy density of 47.5 MJ/L, with an estimated process cost at scale of $8.46-9.14/GJ. Production of high farnesene biofuel from guayule and sorghum on 110 million Ha has the theoretical potential to produce over 30 EJ/yr (30% US annual energy requirement). These crops are thus advantageous because they can provide greater biofuel production on far less acreage and with fewer agronomic inputs than any other current biofuel production system, reduce greenhouse gas emissions, provide energy security to the US and enable US leadership in biofuel production.

The invention provides plant cells and plants to produce β-farnesene and related alkene sesquiterpenes in high yields that can be readily extracted and converted to low-cost liquid biofuels. In some embodiments, mini-chromosome (MC) gene stacking technology is used to advantageously engineer β-farnesene production into plant cells and plants; in further embodiments, such plants are guayule (Parthenium argentatum) and sorghum (Sorghum bicolor). The invention also provides for methods to extract and process farnesene produced by such engineered plant cells and plants into the biofuel molecule farnesane.

### II. Making and Using the Invention

### Note: Definitions are Found at the End of the Detailed Description, Before the Examples

To maximize production of high farnesene, multiple genes are transgenically expressed and that encode proteins that catalyze rate-limiting steps in farnesene production. Furthermore, total carbon flux and re-routing of non-essential carbon into farnesene synthesis by simultaneous regulation of several pathway enzymes and through addition of carbon enhancement technologies is used. Plants with high free carbon stores, such as sorghum genotypes with high-sugar content, high-energy density and photoperiod sensitivity, sugarcane, and guayule genotypes with high resin content and rapid growth, can be used to maximize the flux distribution into the sesquiterpenoid metabolic pathway in some embodiments. To minimize adverse effects of sesquiterpene accumulation on plant growth and development, synthesis of sesquiterpenes is confined to specific cells by the use of tissue-specific promoters for enzyme expression in some embodiments.

The invention also provides for extraction of farnesene from biomass (from plant cells and plants) and efficient processing technology to convert farnesene into the biofuel molecule farnesane. Such engineered plants, such as sorghum and guayule, can be intergressed into elite germplasm or into publically available (and alternatively, improved) lines, to facilitate commercial production.

Genetic Engineering of Increased β-Farnesene Synthesis in Guayule and Sorghum.

Selection of Key Genes for β-Farnesene Metabolic Engineering:

To maximize the production of high β-farnesene terpene resins in plants, such as guayule and sorghum, multiple key pathway enzymes are simultaneously regulated. In order to ensure proper carbon routing to create an effective carbon sink, the invention uses genes encoding proteins catalyzing rate-limiting steps in terpenoid, such as farnesene, production (Table 1, the amino acid sequences of the cited polypeptides are shown in Table 2). In addition to the genes contemplated in Table 1, one of skill in the art will understand that other can be used in addition to those exemplified in Table 1. Furthermore, nucleic acid sequences encoding functional polypeptides, or the active domains, wherein the sequences have sequence identity of at least 70%, 80%, 90%, 95%, 96%, 97%, 98%, or 99% with the proteins listed in Tables 1 and 2. Furthermore, the genomic and non-genomic forms of such sequences can be used. Additionally, plant-optimized polynucleotide sequences can be used, which are generated from the amino acid sequences, for example, shown in Tables 1 and 2; such sequences are codon optimized for expression plants, using for example, the OptimumGene™ Gene Design system (GenScript, New Jersy, USA; see also Burgess-Brown N A, Sharma S, Sobott F, Loenarz C, Oppermann U, Gileadi O. Codon optimization can improve expression of human genes in Escherichia coli: A multi-gene study. Protein Expr Purif. May 2008; 59(1): 94-102). Examples of such plant optimized sequences are shown in Table 3. The polynucleotides shown in Table 3 (SEQ ID NOs:16-27) and those having at least approximately 70%-99% nucleic acid sequence identity to such polynucleotides, including those having at least approximately 70%, 75%, 80%, 81%, 82%, 83%, 84%, 85%, 86%, 87%, 88%, 89%, 90%, 91%, 92%, 93%, 94%, 95%, 96%, 97%, 98%, 99%, and 100% nucleic acid sequence identity to any of SEQ ID NOs:16-27 or to other such codon-optimized sequences, wherein the polypeptide retains the enzymatic activity, can be used.

Genes encoding proteins catalyzing rate-limiting steps and/or the synthesis of crucial intermediates have been identified in both dicot (Arabidopsis) and monocot (rice and maize) systems. These genes are transformed into a plant cells; in some embodiments, the plant cells are from guayule or sorghum, to up-regulate terpenoid synthesis and route carbon into the production of β-farnesene-enriched resins.

TABLE 1
Proteins catalyzing rate-limiting steps in terpenoid production and example proteins from various sources

| | | | Gene ID Number (SEQ | Exemplary |
|---|---|---|---|---|
| | | | ID NO:) (Sequences | Destination |
| Gene | Reaction Catalyzed | Source Organism | found in Table 2) | Species |
| HMG-CoA | Production of HMG-CoA; | Arabidopsis | At1g76490 (1) | Guayule |
| Reductase (3- | rate-limiting step of MVA | (Arabidopsis thaliana) | | |
| hydroxy-3- | pathway | Rice (Oryza sativa) | Os09g0492700 (2) | Sorghum |
| methylglutaryl- | | Brazilian rubber tree | AY706757 (3) | Guayule, |
| coenzyme A | | (Hevea brasiliensis) | | Sorghum |
| reductase) | | | | |
| 1-deoxy-D- | Formation of 1- | Arabidopsis | At4g15560 (4) | Guayule |
| xylulose-5- | deoxy-D-xylulose 5- | (Arabidopsis thaliana) | | |
| phosphate | phosphate (DXP); | Rice (Oryza sativa) | Os05g0408900 (5) | Sorghum |
| synthase (DXS) | rate-limiting step of MEP | Maize (Zea mays) | ABP88134.1 (6) | Guayule, |
| | pathway | | | Sorghum |
| Farnesyl pyro- | Production of FPP | Arabidopsis | At4g17190 (7) | Guayule |
| phosphate | from IPP precursors | (Arabidopsis thaliana) | | |
| synthase (FPPS) | | Rice (Oryza sativa) | Os01g0703400 (8) | Sorghum |
| (farnesyl | | Tomato | AAC73051 (9) | Guayule, |
| diphosphate | | (Solanum lycopersicon) | | Sorghum |
| synthase) | | | | |
| β-Farnesene | Production of β- | Maize (Zea mays) | NP_001105850 (10) | Guayule |
| Synthase | farnesene from FPP | Maize (Zea mays) | NP_001105850 (11; | Sorghum |
| | | | duplicate of 10)) | |
| | | Sweet Wormwood | AY835398 (12) | Guayule, |
| | | (Artemisia annua) | | Sorghum |
| AVP1/OVP1 | Hydrolysis of | AVP1, Arabidopsis | At1g15690 (13) | Guayule |
| | pyrophosphate; | (Arabidopsis thaliana) | | |
| | transport of protons | OVP1, Rice | Os06g0644200 (14) | Sorghum |
| | | (Oryza sativa) | | |
| | | Wheat | AAP55210.1 (15) | Guayule, |
| | | (Triticum aestivum) | | Sorghum |

TABLE 2
Exemplary sequences for proteins catalyzing rate-limiting
steps in terpenoid production

HMG-CoA Reductase)
SEQ ID NO: 1

| | |
|---|---|
| MPSIEVGTVG GGTQLASQSA CLNLLGVKGA STESPGMNAR RLATIVAGAVLAGELSLMSA | 60 |
| IAAGQLVRSH MKYNRSSRDI SGATTTTTTT T | 91 |

SEQ ID NO: 2

| | |
|---|---|
| MAVEGRRRVP LPLPPPTRRG KQQQQQGGER ARRVQAGDAL PLPIRHTNLI FSALFAASLA | 60 |
| YLMRRWREKI RTSTPLHVVG LAEILAICGL VASLIYLLSF FGIAFVQSVV SNSDDEEEEE | 120 |
| DFLIDSRAAG PVAAQATPPP APAPFSLLGS ACAAPKKMPE EDEEIVAEVV AGKIPSYVLE | 180 |
| TRLGDCRRAA GIRREALRRT TGREIRGLPL DGFDYASILG QCCELPVGYV QLPVGVAGPL | 240 |
| VLDGERFYVP MATTEGCLVA STNRGCKAIA ESGGATSVVL QDGMTRAPVA RFPSARRAAE | 300 |
| LKGFLENPAN FDTLAMVFNR SSRFARLQRV KCAVAGRNLY MRFSCSTGDA MGMNMVSKGV | 360 |
| QNVLDYLQDD FPDMDVISIS GNFCSDKKSA AVNWIEGRGK SVVCEAVIKE EVVKKVLKTN | 420 |
| VQSLVELNVI KNLAGSAVAG ALGGFNAHAS NIVTAIFIAT GQDPAQNVES SQCITMLEAV | 480 |
| NDGKDLHISV TMPSIEVGTV GGGTQLASQS ACLDLLGVKG ANRESPGSNA RLLAAVVAGA | 540 |
| VLAGELSLIS AQAAGHLVQS HMKYNRSSKD MSKVAS | 576 |

SEQ ID NO: 3

| | |
|---|---|
| MDTTGRLHHR KHATPVEDRS PTTPKASDAL PLPLYLTNAV FFTLFFSVAY YLLHRWRDKI | 60 |
| RNSTPLHIVT LSEIVAIVSL IASFIYLLGF FGIDFVQSFI ARASHDVWDL EDTDPNYLID | 120 |
| EDHRLVTCPP ANISTKTTII AAPTKLPTSE PLIAPLVSEE DEMIVNSVVD GKIPSYSLES | 180 |
| KLGDCKRAAA IRREALQRMT RRSLEGLPVE GFDYESILGQ CCEMPVGYVQ IPVGIAGPLL | 240 |
| LNGREYSVPM ATTEGCLVAS TNRGCKAIYL SGGATSVLLK DGMTRAPVVR FASATRAAEL | 300 |
| KFFLEDPDNF DTLAVVFNKS SRFARLQGIK CSIAGKNLYI RFSYSTGDAM GMNMVSKGVQ | 360 |
| NVLEFLQSDF SDMDVIGISG NFCSDKKPAA VNWIEGRGKS VVCEAIIKEE VVKKVLKTNV | 420 |
| ASLVELNMLK NLAGSAVAGA LGGFNAHAGN IVSAIFIATG QDPAQNVESS HCITMMEAVN | 480 |
| DGKDLHISVT MPSIEVGTVG GGTQLASQSA CLNLLGVKGA NKESPGSNSR LLAAIVAGSV | 540 |
| LAGELSLMSA IAAGQLVKSH MKYNRSSKDM SKAAS | 575 |
| 1-deoxy-D-xylulose-5-phosphate synthase (DXS) (SEQ ID NOs: 4-6) | |

SEQ ID NO: 4

| | |
|---|---|
| MASSAFAFPS YIITKGGLST DSCKSTSLSS SRSLVTDLPS PCLKPNNNSH SNRRAKVCAS | 60 |
| LAEKGEYYSN RPPTPLLDTI NYPIHMKNLS VKELKQLSDE LRSDVIFNVS KTGGHLGSSL | 120 |
| GVVELTVALH YIFNTPQDKI LWDVGHQSYP HKILTGRRGK MPTMRQTNGL SGFTKRGESE | 180 |
| HDCFGTGHSS TTISAGLGMA VGRDLKGKNN NVVAVIGDGA MTAGQAYEAM NNAGYLDSDM | 240 |
| IVILNDNKQV SLPTATLDGP SPPVGALSSA LSRLQSNPAL RELREVAKGM TKQIGGPMHQ | 300 |
| LAAKVDEYAR GMISGTGSSL FEELGLYYIG PVDGHNIDDL VAILKEVKST RTTGPVLIHV | 360 |
| VTEKGRGYPY AERADDKYHG VVKFDPATGR QFKTTNKTQS YTTYFAEALV AEAEVDKDVV | 420 |
| AIHAAMGGGT GLNLFQRRFP TRCFDVGIAE QHAVTFAAGL ACEGLKPFCA IYSSFMQRAY | 480 |
| DQVVHDVDLQ KLPVRFAMDR AGLVGADGPT HCGAFDVTFM ACLPNMIVMA PSDEADLFNM | 540 |
| VATAVAIDDR PSCFRYPRGN GIGVALPPGN KGVPIEIGKG RILKEGERVA LLGYGSAVQS | 600 |
| CLGAAVMLEE RGLNVTVADA RFCKPLDRAL IRSLAKSHEV LITVEEGSIG GFGSHVVQFL | 660 |
| ALDGLLDGKL KWRPMVLPDR YIDHGAPADQ LAEAGLMPSH IAATALNLIG APREALF | 717 |

SEQ ID NO: 5

| | |
|---|---|
| MALTTFSISR GGFVGALPQE GHFAPAAAEL SLHKLQSRPH KARRRSSSSI SASLSTEREA | 60 |
| AEYHSQRPPT PLLDTVNYPI HMKNLSLKEL QQLADELRSD VIFHVSKTGG HLGSSLGVVE | 120 |
| LTVALHYVFN TPQDKILWDV GHQSYPHKIL TGRRDKMPTM RQTNGLSGFT KRSESEYDSF | 180 |
| GTGHSSTTIS AALGMAVGRD LKGGKNNVVA VIGDGAMTAG QAYEAMNNAG YLDSDMIVIL | 240 |
| NDNKQVSLPT ATLDGPAPPV GALSSALSKL QSSRPLRELR EVAKGVTKQI GGSVHELAAK | 300 |
| VDEYARGMIS GSGSTLFEEL GLYYIGPVDG HNIDDLITIL REVKSTKTTG PVLIHVVTEK | 360 |
| GRGYPYAERA ADKYHGVAKF DPATGKQFKS PAKTLSYTNY FAEALIAEAE QDNRVVAIHA | 420 |
| AMGGGTGLNY FLRRFPNRCF DVGIAEQHAV TFAAGLACEG LKPFCAIYSS FLQRGYDQVV | 480 |
| HDVDLQKLPV RFAMDRAGLV GADGPTHCGA FDVTYMACLP NMVVMAPSDE AELCHMVATA | 540 |
| AAIDDRPSCF RYPRGNGIGV PLPPNYKGVP LEVGKGRVLL EGERVALLGY GSAVQYCLAA | 600 |
| ASLVERHGLK VTVADARFCK PLDQTLIRRL ASSHEVLLTV EEGSIGGFGS HVAQFMALDG | 660 |
| LLDGKLKWRP LVLPDRYIDH GSPADQLAEA GLTPSHIAAT VFNVLGQARE ALAIMTVPNA | 720 |

SEQ ID NO: 6

| | |
|---|---|
| MALSTFSVPR GFLGVPAQDS HFASAVELHV NKLLQARPIN LKPRRRPACV SASLSSEREA | 60 |
| EYYSQRPPTP LLDTINYPVH MKNLSVKELR QLADELRSDV IFHVSKTGGH LGSSLGVVEL | 120 |
| TVALHYVFNA PQDRILWDVG HQSYPHKILT GRRDKMPTMR QTNGLAGFTK RAESEYDSFG | 180 |
| TGHSSTTISA ALGMAVGRDL KGGKNNVVAV IGDGAMTAGQ AYEAMNNAGY LDSDMIVILN | 240 |
| DNKQVSLPTA TLDGPVPPVG ALSSALSKLQ SSRPLRELRE VAKGVTKQIG GSVHELAAKV | 300 |
| DEYARGMISG PGSSLFEELG LYYIGPVDGH NIDDLITILN DVKSTKTTGP VLIHVVTEKG | 360 |
| RGYPYAERAA DKYHGVAKFD PATGKQFKSP AKTLSYTNYF AEALIAEAEQ DSKIVAIHAA | 420 |
| MGGGTGLNYF LRRFPSRCFD VGIAEQHAVT FAAGLACEGL KPFCAIYSSF LQRGYDQVVH | 480 |
| DVDLQKLPVR FAMDRAGLVG ADGPTHCGAF DVAYMACLPN MVVMAPSDEA ELCHMVATAA | 540 |
| AIDDRPSCFR YPRGNGVGVP LPPNYKGTPL EVGKGRILLE GDRVALLGYG SAVQYCLTAA | 600 |
| SLVQRHGLKV TVADARFCKP LDHALIRSLA KSHEVLITVE EGSIGGFGSH IAQFMALDGL | 660 |
| LDGKLKWRPL VLPDRYIDHG SPADQLAEAG LTPSHIAASV FNILGQNREA LAIMAVPNA | 719 |
| Farnesyl pyrophosphate synthase (FPPS) (farnesyl disphosphate | |
| synthase) (SEQ ID NOs: 7-9) | |

SEQ ID NO: 7

| | |
|---|---|
| MADLKSTFLD VYSVLKSDLL QDPSFEFTHE SRQWLERMLD YNVRGGKLNR GLSVVDSYKL | 60 |
| LKQGQDLTEK ETFLSCALGW CIEWLQAYFL VLDDIMDNSV TRRGQPCWFR KPKVGMIAIN | 120 |
| DGILLRNHIH RILKKHFREM PYYVDLVDLF NEVEFQTACG QMIDLITTFD GEKDLSKYSL | 180 |
| QIHRRIVEYK TAYYSFYLPV ACALLMAGEN LENHTDVKTV LVDMGIYFQV QDDYLDCFAD | 240 |
| PETLGKIGTD IEDFKCSWLV VKALERCSEE QTKILYENYG KAEPSNVAKV KALYKELDLE | 300 |
| GAFMEYEKES YEKLTKLIEA HQSKAIQAVL KSFLAKIYKR QK | 342 |

SEQ ID NO: 8

| | |
|---|---|
| MAAAVVANGA SGDSSKAAFA EIYSRLKEEM LEDPAFEFTD ESLQWIDRML DYNVLGGKCN | 60 |
| RGISVIDSFK MLKGTDVLNK EETFLACTLG WCIEWLQAYF LVLDDIMDNS QTRRGQPCWF | 120 |
| RVPQVGLIAV NDGIILRNHI SRILQRHFKG KLYYVDLIDL FNEVEFKTAS GQLLDLITTH | 180 |
| EGEKDLTKYN LTVHRRIVQY KTAYYSFYLP VACALLLSGE NLDNFGDVKN ILVEMGTYFQ | 240 |
| VQDDYLDCYG DPEFIGKIGT DIEDYKCSWL VVQALERADE NQKHILFENY GKPDPECVAK | 300 |
| VKDLYKELNL EAVFHEYERE SYNKLIADIE AHPNKAVQNV LKSFLHKIYK RQK | 353 |

SEQ ID NO: 9

| | |
|---|---|
| MADLKKKFLD VYSVLKSDLL EDTAFEFTDD SRKWVDKMLD YNVPGGKLNR GLSVIDSLSL | 60 |
| LKDGKELTAD EIFKASALGW CIEWLQAYFL VLDDIMDGSH TRRGQPCWYN LEKVGMIAIN | 120 |
| DGILLRNHIT RILKKYFRPE SYYVDLLDLF NEVEFQTASG QMIDLITTLV GEKDLSKYSL | 180 |
| SIHRRIVQYK TAYYSFYLPV ACALLMVGEN LDKHVDVKKI LIDMGIYFQV QDDYLDCFAD | 240 |
| PEVLGKIGTD IQDFKCSWLV VKALELCNEE QKKILFENYG KDNAACIAKI KALYNDLKLE | 300 |
| EVFLEYEKTS YEKLTTSIAA HPSKAVQAVL LSFLGKIYKR QK | 342 |
| β-Farnesene Synthase (SEQ ID NOs: 10-12) | |

SEQ ID NOs: 10 and 11

| | |
|---|---|
| MDATAFHPSL WGDFFVKYKP PTAPKRGHMT ERAELLKEEV RKTLKAAANQ ITNALDLIIT | 60 |
| LQRLGLDHHY ENEISELLRF VYSSSDYDDK DLYVVSLRFY LLRKHGHCVS SDVFTSFKDE | 120 |
| EGNFVVDDTK CLLSLYNAAY VRTHGEKVLD EAITFTRRQL EASLLDPLEP ALADEVHLTL | 180 |
| QTPLFRRLRI LEAINYIPIY GKEAGRNEAI LELAKLNFNL AQLIYCEELK EVTLWWKQLN | 240 |
| VETNLSFIRD RIVECHFWMT GACCEPQYSL SRVIATKMTA LITVLDDMMD TYSTTEEAML | 300 |
| LAEAIYRWEE NAAELLPRYM KDFYLYLLKT IDSCGDELGP NRSFRTFYLK EMLKVLVRGS | 360 |
| SQEIKWRNEN YVPKTISEHL EHSGPTVGAF QVACSSFVGM GDSITKESFE WLLTYPELAK | 420 |
| SLMNISRLLN DTASTKREQN AGQHVSTVQC YMLKHGTTMD EACEKIKELT EDSWKDMMEL | 480 |
| YLTPTEHPKL IAQTIVDFAR TADYMYKETD GFTFSHTIKD MIAKLFVDPI SLF | 533 |

SEQ ID NO: 12

| | |
|---|---|
| MSTLPISSVS FSSSTSPLVV DDKVSTKPDV IRHTMNFNAS IWGDQFLTYD EPEDLVMKKQ | 60 |
| LVEELKEEVK KELITIKGSN EPMQHVKLIE LIDAVQRLGI AYHFEEEIEE ALQHIHVTYG | 120 |
| EQWVDKENLQ SISLWFRLLR QQGFNVSSGV FKDFMDEKGK FKESLCNDAQ GILALYEAAF | 180 |
| MRVEDETILD NALEFTKVHL DIIAKDPSCD SSLRTQIHQA LKQPLRRRLA RIEALHYMPI | 240 |
| YQQETSHDEV LLKLAKLDFS VLQSMHKKEL SHICKWWKDL DLQNKLPYVR DRVVEGYFWI | 300 |
| LSIYYEPQHA RTRMFLMKTC MWLVVLDDTF DNYGTYEELE IFTQAVERWS ISCLDMLPEY | 360 |
| MKLIYQELVN LHVEMEESLE KEGKTYQIHY VKEMAKELVR NYLVEARWLK EGYMPTLEEY | 420 |
| MSVSMVTGTY GLMIARSYVG RGDIVTEDTF KWVSSYPPII KASCVIVRLM DDIVSHKEEQ | 480 |
| ERGHVASSIE CYSKESGASE EEACEYISRK VEDAWKVINR ESLRPTAVPF PLLMPAINLA | 540 |
| RMCEVLYSVN DGFTHAEGDM KSYMKSFFVH PMVV | 574 |
| AVP1/OVP1 (SEQ ID NOs: 13-15) | |

SEQ ID NO: 13

| | |
|---|---|
| MVAPALLPEL WTEILVPICA VIGIAFSLFQ WYVVSRVKLT SDLGASSSGG ANNGKNGYGD | 60 |
| YLIEEEEGVN DQSVVAKCAE IQTAISEGAT SFLFTEYKYV GVFMIFFAAV IFVFLGSVEG | 120 |
| FSTDNKPCTY DTTRTCKPAL ATAAFSTIAF VLGAVTSVLS GFLGMKIATY ANARTTLEAR | 180 |
| KGVGKAFIVA FRSGAVMGFL LAASGLLVLY ITINVFKIYY GDDWEGLFEA ITGYGLGGSS | 240 |
| MALFGRVGGG IYTKAADVGA DLVGKIERNI PEDDPRNPAV IADNVGDNVG DIAGMGSDLF | 300 |
| GSYAEASCAA LVVASISSFG INHDFTAMCY PLLISSMGIL VCLITTLFAT DFFEIKLVKE | 360 |
| IEPALKNQLI ISTVIMTVGI AIVSWVGLPT SFTIFNFGTQ KVVKNWQLFL CVCVGLWAGL | 420 |
| IIGFVTEYYT SNAYSPVQDV ADSCRTGAAT NVIFGLALGY KSVIIPIFAI AISIFVSFSF | 480 |
| AAMYGVAVAA LGMLSTIATG LAIDAYGPIS DNAGGIAEMA GMSHRIRERT DALDAAGNTT | 540 |
| AAIGKGFAIG SAALVSLALF GAFVSRAGIH TVDVLTPKVI IGLLVGAMLP YWFSAMTMKS | 600 |
| VGSAALKMVE EVRRQFNTIP GLMEGTAKPD YATCVKISTD ASIKEMIPPG CLVMLTPLIV | 660 |
| GFFFGVETLS GVLAGSLVSG VQIAISASNT GGAWDNAKKY IEAGVSEHAK SLGPKGSEPH | 720 |
| KAAVIGDTIG DPLKDTSGPS LNILIKLMAV ESLVFAPFFA THGGILFKYF | 770 |

SEQ ID NO: 14

| | |
|---|---|
| MNPSARISQV AMAAILPDLA TQVLVPAAAV VGIAFAVVQW VLVSKVKMTA ERRGGEGSPG | 60 |
| AAAGKDGGAA SEYLIEEEEG LNEHNVVEKC SEIQHAISEG ATSFLFTEYK YVGLFMGIFA | 120 |
| VLIFLFLGSV EGFSTKSQPC HYSKDRMCKP ALANAIFSTV AFVLGAVTSL VSGFLGMKIA | 180 |
| TYANARTTLE ARKGVGKAFI TAFRSGAVMG FLLAASGLVV LYIAINLFGI YYGDDWEGLF | 240 |
| EAITGYGLGG SSMALFGRVG GGIYTKAADV GADLVGKVER NIPEDDPRNP AVIADNVGDN | 300 |
| VGDIAGMGSD LFGSYAESSC AALVVASISS FGINHEFTPM LYPLLISSVG IIACLITTLF | 360 |
| ATDFFEIKAV DEIEPALKKQ LIISTVVMTV GIALVSWLGL PYSFTIFNFG AQKTVYNWQL | 420 |
| FLCVAVGLWA GLIIGFVTEY YTSNAYSPVQ DVADSCRTGA ATNVIFGLAL GYKSVIIPIF | 480 |
| AIAFSIFLSF SLAAMYGVAV AALGMLSTIA TGLAIDAYGP ISDNAGGIAE MAGMSHRIRE | 540 |
| RTDALDAAGN TTAAIGKGFA IGSAALVSLA LFGAFVSRAA ISTVDVLTPK VFIGLIVGAM | 600 |
| LPYWFSAMTM KSVGSAALKM VEEVRRQFNS IPGLMEGTTK PDYATCVKIS TDASIKEMIP | 660 |
| PGALVMLSPL IVGIFFGVET LSGLLAGALV SGVQIAISAS NTGGAWDNAK KYIEAGASEH | 720 |
| ARTLGPKGSD CHKAAVIGDT IGDPLKDTSG PSLNILIKLM AVESLVFAPF FATHGGILFK | 780 |
| WF | 782 |

SEQ ID NO: 15

| | |
|---|---|
| MAILGELGTE ILIPVCGVVG IVFAVAQWFI VSKVKVTPGA ASAAGGGKNG YGDYLIEEEE | 60 |
| GLNDHNVVVK CAEIQTAISE GATSFLFTMY QYVGMFMVVF AAVIFVFLGS IEGFSTKGQP | 120 |
| CTYSTGTCKP ALYTALFSTA SFLLGAITSL VSGFLGMKIA TYANARTTLE ARKGVGKAFI | 180 |
| TAFRSGAVMG FLLSSSGLGV LYITINVFKM YYGDDWEGLF ESITGYGLGG SSMALFGRVG | 240 |
| GGIYTKAADV GADLVGKVER NIPEDGPRNP AVIADNVGDN VGDIAGMGSD LFGSYAESSC | 300 |
| AALVVASISS FGINHDFTAM CYPLLVSSVG IIVCLLTTLF ATDFFEIKAA SEIEPALKKQ | 360 |
| LIIFTALMTI GVAVINWLAL PAKFTIFNFG AQKDVSNWGL FFCVAVGLWA GLIIGFVTEY | 420 |
| YTSNAYSPVQ DVADSCRTGA ATNVIFGLAL GYKSVIIPIF AIAVSIYVSF SIAAMYGIAM | 480 |
| AALGMLSTTA TGLAIDAYGP ISDNAGGIAE MAGMSHRIRE RTDALDAAGN TTAAIGKGFA | 540 |
| IGSAALVSLA LFGAFVSRAG VKVVDVLSPK VFIGLIVGAM LPYWFSAMTR RVCESAALKM | 600 |
| VEKVRRQFNT IPGLMKGTAK PDYATCVKIS TDASIREMIP PGALVMLTPL IVGTLFGVET | 660 |
| LSGVLAGALV SGVQIAISAS NTGGAWDNAK KYIEAGNSEH ARSLGPKGSD CHKAAVIGDT | 720 |
| IGDPLKDTSG PSLNILIKLM AVESLVFAPF FATYGGVLFK YI | 762 |

TABLE 3
Examples of plant-optimized polynucleotide sequences

HMG CoA reductase (3-hydroxy-3-methylglutaryl coenzyme A reductase)
(3 examples; (3-hydroxy-3-methylglutaryl-coenzyme A reductase)
(SEQ ID NOs: 1-3; SEQ ID NO: 28 is based on Saccharomyces cerevisiae
polypeptide sequence)

SEQ ID NO: 16

| | |
|---|---|
| GGATCCGAGC TCATGGATGT TAGGAGAAGA CCAACCAGCG GCAAGACGAT TCATTCCGTT | 60 |
| AAGCCCAAGT CAGTGGAGGA CGAGTCGGCA CAGAAGCCCT CCGACGCCTT GCCACTCCCG | 120 |
| CTGTACCTTA TCAACGCTCT CTGCTTCACA GTGTTCTTTT ACGTGGTCTA TTTTCTCCTG | 180 |
| TCGCGGTGGA GAGAAAAGAT TCGCACGTCC ACTCCCCTTC ACGTTGTGGC TTTGAGCGAG | 240 |
| ATCGCCGCTA TTGTCGCGTT CGTTGCATCT TTTATCTATC TTTTGGGGTT CTTTGGTATC | 300 |
| GATTTCGTCC AGTCATTGAT TCTCCGGCCA CCGACGGACA TGTGGGCCGT TGACGATGAC | 360 |
| GAGGAAGAGA CAGAAGAGGG CATTGTGCTC CGGGAGGATA CGAGAAAGCT GCCGTGCGGG | 420 |
| CAAGCCCTTG ACTGTTCATT GTCGGCGCCT CCCCTCTCTA GGGCAGTCGT TTCCAGCCCC | 480 |
| AAGGCCATGG ACCCAATCGT CCTGCCTAGC CCCAAGCCAA AGGTTTTCGA CGAAATTCCG | 540 |
| TTTCCTACCA CAACGACTAT CCCCATTCTC GGCGATGAGG ACGAAGAGAT CATTAAGTCG | 600 |
| GTGGTCGCGG GCACTATCCC ATCCTACAGC CTCGAATCCA AGCTGGGGGA TTGCAAGAGA | 660 |
| GCAGCAGCAA TCAGGAGAGA GGCACTCCAG AGGATTACCG GAAAGTCTCT GTCAGGCCTG | 720 |
| CCCCTTGAAG GGTTCGACTA CGAGAGCATC CTGGGCCAGT GCTGTGAGAT GCCAGTGGGG | 780 |
| TATGTCCAAA TCCCGGTGGG AATTGCCGGC CCTCTCCTGC TTGATGGCAA GGAATATAGC | 840 |
| GTGCCAATGG CCACCACAGA GGGTTGCCTG GTCGCTTCTA CCAACCGCGG CTGTAAGGCC | 900 |
| ATCCATCTTT CCGGAGGAGC TACGAGCGTC TTGCTCAGGG ATGGCATGAC TAGGGCCCCA | 960 |
| GTTGTGCGGT TCGGGACCGC AAAGAGAGCT GCACAGTTGA AGCTCTACCT GGAAGACCCT | 1020 |
| GCCAACTTTG AGACCCTCTC GACATCCTTC AATAAGTCTT CAAGGTTTGG TCGCCTTCAA | 1080 |
| TCCATCAAGT GCGCAATTGC CGGAAAGAAT CTCTATATGC GCTTCTGCTG TTCTACAGGG | 1140 |
| GACGCCATGG GTATGAACAT GGTGTCAAAG GGCGTTCAGA ACGTGCTCAA TTTCCTGCAA | 1200 |
| AATGATTTTC CGGATATGGA CGTGATCGGG CTGTCTGGTA ACTTCTGCTC AGACAAGAAG | 1260 |
| CCTGCAGCCG TCAATTGGAT TGAAGGAAGG GGCAAGAGCG TCGTTTGTGA GGCGATCATT | 1320 |
| AAGGGCGACG TGGTCAAGAA GGTGCTCAAG ACTAACGTGG AAGCACTTGT CGAGTTGAAC | 1380 |
| ATGCTCAAGA ATCTGACCGG TTCAGCTATG GCGGGAGCAC TGGGTGGATT CAACGCCCAC | 1440 |
| GCTTCGAATA TCGTCACCGC CATCTACATT GCTACAGGCC AGGACCCAGC GCAAAACGTC | 1500 |
| GAATCGTCCA ATTGCATCAC AATGATGGAG GCAGTTAATG ATGGTCAGGA CCTCCATGTT | 1560 |
| TCGGTGACGA TGCCATCCAT TGAGGTCGGC ACGGTTGGCG GGGGTACTCA GCTTGCGAGC | 1620 |
| CAATCTGCAT GTTTGAACCT GCTTGGAGTG AAGGGAGCAT CCAAGGAGAC CCCAGGTGCA | 1680 |
| AATAGCAGAG TCCTTGCCTC TATCGTTGCT GGATCAGTGT TGGCTGCGGA GCTTTCATTG | 1740 |
| ATGTCGGCCA TTGCAGCCGG CCAGCTGGTT AACTCCCACA TGAAGTACAA CAGGGCTAAT | 1800 |
| AAGGAGGCTG CGGTCAGCAA GCCTAGCTCT TGAGGTACCT CTAGAAAGCT T | 1851 |

SEQ ID NO: 17

| | |
|---|---|
| GGATCCGAGC TCATGGCTGC CGATCAACTG GTGAAGACCG AGGTTACTAA GAAGTCGTTT | 60 |
| ACTGCCCCTG TCCAAAAGGC GTCCACTCCC GTGCTGACCA ACAAGACCGT TATCTCGGGT | 120 |
| TCCAAGGTGA AGTCCCTCTC CAGCGCCCAG TCTTCATCGT CCGGACCATC CTCCTCCTCC | 180 |
| GAGGAAGACG ATTCGCGGGA CATCGAGTCC CTGGATAAGA AGATTAGACC TCTCGAGGAA | 240 |
| CTGGAAGCCC TCCTGTCCAG CGGCAACACA AAGCAACTCA AGAATAAGGA GGTTGCCGCT | 300 |
| CTCGTGATCC ACGGCAAGCT CCCCTTGTAC GCTCTTGAAA AGAAGTTGGG AGACACCACA | 360 |
| AGGGCGGTTG CAGTGAGGCG CAAGGCGCTT TCGATTTTGG CCGAGGCTCC GGTGCTCGCA | 420 |
| TCAGATAGGC TGCCTTATAA GAACTACGAC TATGATCGCG TGTTCGGCGC CTGCTGTGAG | 480 |
| AATGTCATCG GGTACATGCC ACTTCCGGTC GGTGTTATCG GACCCCTCGT GATCGACGGC | 540 |
| ACATCTTATC ATATCCCAAT GGCGACGACT GAGGGTTGCC TCGTCGCAAG CGCAATGAGA | 600 |
| GGCTGTAAGG CCATTAACGC TGGCGGGGGT GCAACCACAG TGCTGACTAA GGACGGTATG | 660 |
| ACCAGGGGAC CAGTGGTCCG CTTCCCTACG CTTAAGCGCT CTGGCGCCTG CAAGATTTGG | 720 |
| CTCGATTCAG AGGAAGGGCA GAACGCGATT AAGAAGGCAT TCAATAGCAC ATCTAGGTTT | 780 |
| GCGCGCCTCC AGCACATCCA AACGTGTCTG GCAGGTGACC TTTTGTTCAT GCGGTTTAGA | 840 |
| ACAACTACCG GCGATGCTAT GGGGATGAAT ATGATTTCAA AGGGCGTTGA GTACTCGCTC | 900 |
| AAGCAAATGG TGGAGGAATA TGGTTGGGAG GACATGGAAG TTGTGTCAGT GTCGGGAAAC | 960 |
| TACTGCACTG ATAAGCCCGC GGCAATCAAT TGGATTGAGG GAAGGGGGAA GTCCGTCGTT | 1020 |
| GCAGAAGCTA CCATCCCAGG CGACGTGGTC AGAAAGGTCC TGAAGTCTGA TGTCTCAGCC | 1080 |
| CTCGTTGAGC TGAACATTGC TAAGAATCTT GTCGGTAGCG CGATGGCAGG ATCTGTTGGA | 1140 |
| GGCTTCAACG CCCATGCCGC TAATCTGGTG ACAGCCGTCT TTCTCGCTCT GGGCCAGGAC | 1200 |
| CCTGCTCAAA ACGTGGAGTC TTCAAATTGC ATCACGCTCA TGAAGGAAGT CGACGGGGAT | 1260 |
| CTGCGGATTT CCGTCAGCAT GCCGAGCATC GAGGTTGGCA CAATTGGGGG TGGAACGGTT | 1320 |
| CTTGAACCTC AGGGGGCGAT GTTGGATCTC CTGGGCGTCA GAGGACCACA CGCAACAGCT | 1380 |
| CCAGGCACGA ACGCGCGGCA ACTCGCAAGA ATCGTGGCAT GCGCAGTCCT GGCAGGAGAG | 1440 |
| CTTTCCTTGT GTGCGGCACT TGCCGCTGGG CATTTGGTGC AGAGCCACAT GACTCATAAC | 1500 |
| AGGAAGCCTG CCGAGCCCAC TAAGCCAAAC AATCTTGACG CTACCGATAT CAATCGCTTG | 1560 |
| AAGGACGGCT CCGTCACCTG CATTAAGAGC TAAGGTACCA AGCTT | 1605 |

SEQ ID NO: 28

| | |
|---|---|
| GGATCCGAGC TCATGGATGT TAGGAGAAGA CCAACCAGCG GCAAGACGAT TCATTCCGTT | 60 |
| AAGCCCAAGT CAGTGGAGGA CGAGTCGGCA CAGAAGCCCT CCGACGCCTT GCCACTCCCG | 120 |
| CTGTACCTTA TCAACGCTCT CTGCTTCACA GTGTTCTTTT ACGTGGTCTA TTTTCTCCTG | 180 |
| TCGCGGTGGA GAGAAAAGAT TCGCACGTCC ACTCCCCTTC ACGTTGTGGC TTTGAGCGAG | 240 |
| ATCGCCGCTA TTGTCGCGTT CGTTGCATCT TTTATCTATC TTTTGGGGTT CTTTGGTATC | 300 |
| GATTTCGTCC AGTCATTGAT TCTCCGGCCA CCGACGGACA TGTGGGCCGT TGACGATGAC | 360 |
| GAGGAAGAGA CAGAAGAGGG CATTGTGCTC CGGGAGGATA CGAGAAAGCT GCCGTGCGGG | 420 |
| CAAGCCCTTG ACTGTTCATT GTCGGCGCCT CCCCTCTCTA GGGCAGTCGT TTCCAGCCCC | 480 |
| AAGGCCATGG ACCCAATCGT CCTGCCTAGC CCCAAGCCAA AGGTTTTCGA CGAAATTCCG | 540 |
| TTTCCTACCA CAACGACTAT CCCCATTCTC GGCGATGAGG ACGAAGAGAT CATTAAGTCG | 600 |
| GTGGTCGCGG GCACTATCCC ATCCTACAGC CTCGAATCCA AGCTGGGGGA TTGCAAGAGA | 660 |
| GCAGCAGCAA TCAGGAGAGA GGCACTCCAG AGGATTACCG GAAAGTCTCT GTCAGGCCTG | 720 |
| CCCCTTGAAG GGTTCGACTA CGAGAGCATC CTGGGCCAGT GCTGTGAGAT GCCAGTGGGG | 780 |
| TATGTCCAAA TCCCGGTGGG AATTGCCGGC CCTCTCCTGC TTGATGGCAA GGAATATAGC | 840 |
| GTGCCAATGG CCACCACAGA GGGTTGCCTG GTCGCTTCTA CCAACCGCGG CTGTAAGGCC | 900 |
| ATCCATCTTT CCGGAGGAGC TACGAGCGTC TTGCTCAGGG ATGGCATGAC TAGGGCCCCA | 960 |
| GTTGTGCGGT TCGGGACCGC AAAGAGAGCT GCACAGTTGA AGCTCTACCT GGAAGACCCT | 1020 |
| GCCAACTTTG AGACCCTCTC GACATCCTTC AATAAGTCTT CAAGGTTTGG TCGCCTTCAA | 1080 |
| TCCATCAAGT GCGCAATTGC CGGAAAGAAT CTCTATATGC GCTTCTGCTG TTCTACAGGG | 1140 |
| GACGCCATGG GTATGAACAT GGTGTCAAAG GGCGTTCAGA ACGTGCTCAA TTTCCTGCAA | 1200 |
| AATGATTTTC CGGATATGGA CGTGATCGGG CTGTCTGGTA ACTTCTGCTC AGACAAGAAG | 1260 |
| CCTGCAGCCG TCAATTGGAT TGAAGGAAGG GGCAAGAGCG TCGTTTGTGA GGCGATCATT | 1320 |
| AAGGGCGACG TGGTCAAGAA GGTGCTCAAG ACTAACGTGG AAGCACTTGT CGAGTTGAAC | 1380 |
| ATGCTCAAGA ATCTGACCGG TTCAGCTATG GCGGGAGCAC TGGGTGGATT CAACGCCCAC | 1440 |
| GCTTCGAATA TCGTCACCGC CATCTACATT GCTACAGGCC AGGACCCAGC GCAAAACGTC | 1500 |
| GAATCGTCCA ATTGCATCAC AATGATGGAG GCAGTTAATG ATGGTCAGGA CCTCCATGTT | 1560 |
| TCGGTGACGA TGCCATCCAT TGAGGTCGGC ACGGTTGGCG GGGGTACTCA GCTTGCGAGC | 1620 |
| CAATCTGCAT GTTTGAACCT GCTTGGAGTG AAGGGAGCAT CCAAGGAGAC CCCAGGTGCA | 1680 |
| AATAGCAGAG TCCTTGCCTC TATCGTTGCT GGATCAGTGT TGGCTGCGGA GCTTTCATTG | 1740 |
| ATGTCGGCCA TTGCAGCCGG CCAGCTGGTT AACTCCCACA TGAAGTACAA CAGGGCTAAT | 1800 |
| AAGGAGGCTG CGGTCAGCAA GCCTAGCTCT TGAGGTACCT CTAGAAAGCT T | 1851 |
| 1-deoxy-D-xyulose-5-phosphate synthase (3 examples) | |
| (with chloroplast targeting sequence) | |

SEQ ID NO: 18

| | |
|---|---|
| GGATCCGAGC TCATGGCGTT GACTACATTT TCGATTTCAC GGGGGGGTTT CGTTGGAGCC | 60 |
| CTGCCGCAAG AAGGACACTT TGCACCTGCC GCTGCTGAGC TTTCGTTGCA CAAGCTGCAG | 120 |
| TCCCGGCCTC ATAAGGCAAG GAGACGGTCC AGCTCTTCAA TCAGCGCATC TCTCTCAACG | 180 |
| GAGCGGGAAG CCGCTGAGTA CCACTCTCAA AGACCACCGA CGCCTCTCCT GGACACTGTG | 240 |
| AACTATCCCA TCCATATGAA GAATCTCAGC CTGAAGGAGC TTCAGCAATT GGCGGACGAA | 300 |
| CTGCGCTCCG ATGTCATTTT CCACGTTAGC AAGACGGGCG GGCATCTTGG ATCGTCCTTG | 360 |
| GGAGTGGTCG AGCTGACGGT GGCACTGCAC TACGTCTTTA ACACTCCGCA GGACAAGATC | 420 |
| CTCTGGGATG TCGGACACCA ATCCTATCCT CATAAGATTC TGACTGGCAG AAGGGACAAG | 480 |
| ATGCCCACGA TGAGGCAGAC TAATGGTCTC TCCGGATTCA CCAAGCGCTC GGAGTCCGAA | 540 |
| TACGATTCGT TTGGAACAGG CCATAGCTCT ACCACAATCT CCGCAGCATT GGGAATGGCA | 600 |
| GTGGGTAGGG ACCTCAAGGG TGGAAAGAAC AATGTTGTGG CAGTCATTGG GGATGGTGCG | 660 |
| ATGACCGCAG GACAGGCCTA CGAGGCTATG AACAATGCCG GCTATCTGGA CAGCGATATG | 720 |
| ATCGTTATTC TTAACGACAA TAAGCAAGTG TCTCTGCCTA CCGCAACACT TGATGGACCA | 780 |
| GCACCTCCAG TGGGTGCGCT GTCATCGGCA CTCAGCAAGC TGCAGTCCAG CCGCCCTCTT | 840 |
| CGGGAGTTGA GAGAAGTGGC CAAGGGCGTC ACCAAGCAAA TCGGCGGGTC CGTTCACGAG | 900 |
| CTGGCCGCTA AGGTGGACGA ATACGCTCGG GGGATGATTA GCGGATCTGG CTCAACACTC | 960 |
| TTCGAGGAAC TTGGCTTGTA CTATATCGGA CCCGTGGATG GCCATAACAT TGACGATCTT | 1020 |
| ATCACGATTT TGAGAGAGGT GAAGTCCACT AAGACGACTG GCCCAGTCCT CATCCACGTC | 1080 |
| GTTACGGAGA AGGGGAGGGG TTACCCGTAT GCGGAACGCG CGGCAGACAA GTACCATGGG | 1140 |
| GTCGCGAAGT TCGATCCAGC AACTGGCAAG CAGTTTAAGA GCCCGGCAAA GACCTTGTCT | 1200 |
| TACACAAACT ATTTCGCCGA GGCTCTTATC GCGGAGGCAG AACAAGACAA TAGGGTGGTC | 1260 |
| GCTATTCACG CAGCTATGGG TGGAGGCACC GGCCTCAACT ATTTCCTGCG CCGGTTTCCA | 1320 |
| AATCGCTGCT TCGATGTCGG CATCGCCGAG CAGCATGCTG TTACATTTGC GGCAGGATTG | 1380 |
| GCCTGCGAAG GCCTCAAGCC GTTCTGTGCT ATCTACTCTT CATTTCTGCA GAGGGGCTAT | 1440 |
| GACCAAGTTG TGCACGACGT CGATCTCCAG AAGCTGCCTG TTCGGTTCGC GATGGACAGA | 1500 |
| GCAGGACTCG TCGGAGCTGA TGGTCCAACC CATTGCGGAG CCTTTGACGT TACATACATG | 1560 |
| GCTTGTCTTC CAAACATGGT CGTTATGGCC CCGTCCGATG AGGCTGAACT CTGCCACATG | 1620 |
| GTGGCAACCG CAGCTGCAAT CGACGATAGA CCAAGCTGTT TCCGCTACCC ACGCGGAAAC | 1680 |
| GGCATTGGGG TCCCTCTGCC ACCGAATTAT AAGGGCGTTC CCCTTGAGGT CGGCAAGGGA | 1740 |
| CGGGTGCTTT TGGAGGGTGA AAGAGTCGCG CTCCTGGGCT ACGGGTCTGC AGTTCAGTAT | 1800 |
| TGCCTGGCAG CCGCTTCACT TGTGGAGAGA CACGGACTGA AGGTGACGGT CGCCGACGCT | 1860 |
| AGATTCTGTA AGCCACTTGA TCAAACTTTG ATCAGAAGGC TCGCCTCGTC CCACGAGGTC | 1920 |
| CTTTTGACCG TTGAGGAAGG ATCAATTGGG GGTTTCGGCT CGCATGTGGC CCAGTTTATG | 1980 |
| GCTTTGGACG GGCTCCTGGA TGGCAAGCTC AAGTGGAGGC CTCTCGTCCT GCCCGACCGC | 2040 |
| TACATCGATC ACGGGTCACC AGCAGACCAG TTGGCAGAGG CAGGTCTCAC CCCGTCGCAT | 2100 |
| ATCGCGGCAA CAGTTTTCAA CGTGCTGGGA CAAGCAAGAG AAGCCCTTGC TATTATGACA | 2160 |
| GTGCCGAATG CTTGAGGTAC CTCTAGAAAG CTT | 2193 |

SEQ ID NO: 19

| | |
|---|---|
| GGATCCGAGC TCATGGCCCT CTCTGCGTGT TCGTTCCCT GCTCATGTTGA CAAGGCGACT | 60 |
| ATCAGCGACC TCCAAAAGTA TGGTTATGTG CCCAGCCGC AGCCTCTGGAG AACGGACCTC | 120 |
| CTGGCCCAGA GCTTGGGAAG GCTCAACCAG GCTAAGTCT AAGAAGGGACC TGGAGGAATC | 180 |
| TGCGCTTCCC TGAGCGAGAG AGGCGAATAC CACTCACAG AGGCCACCGAC TCCTCTTTTG | 240 |
| GACACCACAA ACTATCCCAT CCATATGAAG AATCTTAGC ATTAAGGAGCT GAAGCAACTT | 300 |
| GCCGACGAAT TGCGCTCGGA TGTGATCTTC AACGTCTCC CGGACGGGTGG ACACTTGGGC | 360 |
| TCCTCCCTCG GAGTGGTCGA GCTGACTGTT GCGCTTCAT TACGTGTTCTC AGCACCTCGG | 420 |
| GACAAGATCC TTTGGGATGT GGGGCACCAG TCCTACCCC CATAAGATCCT CACCGGTAGG | 480 |
| CGCGAGAAGA TGTATACGAT TCGCCAAACT AATGGCCTC TCTGGGTTCAC CAAGCGGTCT | 540 |
| GAGTCAGAAT ACGACTGCTT TGGAACAGGC CACTCTTCA ACGACTATCTC CGCAGGACTC | 600 |
| GGTATGGCAG TGGGAAGGGA CCTGAAGGGC AAGAAGAAC AACGTTGTGGC AGTCATTGGA | 660 |
| GATGGCGCGA TGACAGCAGG GCAGGCCTAC GAGGCTATG AACAATGCCGG TTATCTTGAC | 720 |
| TCAGATATGA TCGTTATCTT GAACGACAAT AAGCAAGTG TCGCTCCCTAC CGCCACACTG | 780 |
| GATGGACCAA TCCCTCCAGT GGGCGCGCTG TCGTCCGCA TTGTCGAGACT CCAGTCCAAC | 840 |
| AGGCCTCTGC GCGAGCTTCG GGAAGTTGCA AAGGGCGTG ACCAAGCAAAT CGGAGGACCA | 900 |
| ATGCACGAGT GGGCAGCTAA GGTGGACGAA TACGCCCGC GGCATGATTTC GGGGTCCGGT | 960 |
| AGCACACTCT TCGAGGAACT TGGCTTGTAC TATATCGGG CCTGTCGATGG TCATAATATT | 1020 |
| GACGATTTGA TCGCTATTCT CAAGGAGGTG AAGTCCACG AAGACCACAGG CCCAGTCCTG | 1080 |
| ATCCACGTCG TTACTGAGAA GGGACGCGGC TACCCGTAT GCGGAAAAGGC GGCAGACAAG | 1140 |
| TACCATGGCG TCACCAAGTT CGATCCCGCG ACAGGAAAG CAGTTTAAGGG CTCAGCAATC | 1200 |
| ACGCAATCGT ACACGACTTA TTTCGCCGAG GCTCTCATT GCGGAGGCAGA AGTCGACAAG | 1260 |
| GATATCGTTG CCATTCACGC AGCTATGGGT GGAGGCACG GGGCTCAACCT GTTCCTTCGG | 1320 |
| AGATTTCCAA CTCGCTGCTT CGACGTCGGC ATCGCCGAG CAGCATGCTGT TACCTTTGCG | 1380 |
| GCAGGGCTTG CCTGCGAAGG TTTGAAGCCG TTCTGTGCT ATCTACAGCTC TTTTATGCAG | 1440 |
| CGGGCGTATG ATCAAGTGGT CCACGACGTG GATTTGCAG AAGCTCCCAGT CCGCTTCGCG | 1500 |
| ATGGACAGAG CAGGTCTCGT GGGAGCAGAT GGACCAACC CATTGCGGAGC ATTCGACGTC | 1560 |
| ACCTTCATGG CTTGTCTGCC AAATATGGTT GTGATGGCC CCGAGCGATGA GGCTGAACTT | 1620 |
| TTCCACATGG TGGCAACCGC AGCTGCAATC GACGATAGA CCATCTTGTTT TAGATACCCG | 1680 |
| AGGGGGAACG GTGTCGGAGT TCAGCTGCCA CCGGGGAAT AAGGGTATTCC GCTCGAGGTC | 1740 |
| GGCAAGGGAC GCATCCTGAT TGAGGGCGAA CGGGTTGCG CTCCTGGGTTA TGGAACCGCA | 1800 |
| GTGCAGTCCT GCCTCGCAGC AGCTAGCCTG GTCGAGCCT CACGGCCTTTT GATCACCGTT | 1860 |
| GCCGACGCTA GATTCTGTAA GCCCCTGGAT CACACACTT ATTAGGAGCTT GGCCAAGTCT | 1920 |
| CATGAGGTCC TCATCACAGT TGAGGAAGGG TCTATTGGG GGTTTCGGTTC ACACGTGGCC | 1980 |
| CACTTCCTCG CTCTCGACGG ACTCCTGGAT GGCAAGCTG AAGTGGAGACC TCTGGTTCTT | 2040 |
| CCCGACAGGT ACATCGATCA CGGATCTCCA TCAGTCCAG CTTATTGAGGC TGGATTGACG | 2100 |
| CCAAGCCATG TGGCAGCAAC TGTCCTGAAC ATCCTTGGC AATAAGAGGGA AGCGCTGCAA | 2160 |
| ATTATGTCAT CGTGAGGTAC CTCTAGAAAG CTT | 2193 |
| (with chloroplast targeting sequence) | |

SEQ ID NO: 20

| | |
|---|---|
| GGATCCGAGC TCATGGCGTT GACTACATTT TCGATTTCAC GGGGGGGTTT CGTTGGAGCC | 60 |
| CTGCCGCAAG AAGGACACTT TGCACCTGCC GCTGCTGAGC TTTCGTTGCA CAAGCTGCAG | 120 |
| TCCCGGCCTC ATAAGGCAAG GAGACGGTCC AGCTCTTCAA TCAGCGCGTC TCTGTCAGAG | 180 |
| AGAGGCGAAT ACCACAGCCA GAGGCCACCG ACACCTCTTT TGGACACGAC TAACTATCCC | 240 |
| ATCCATATGA AGAATCTTTC TATTAAGGAG CTGAAGCAAC TTGCCGACGA ACTCCGCTCC | 300 |
| GATGTGATCT TCAACGTCAG CCGGACCGGA GGACACTTGG GGTCCAGCCT CGGTGTGGTC | 360 |
| GAGCTGACAG TTGCGCTTCA TTACGTGTTC AGCGCACCTC GCGACAAGAT CCTGTGGGAT | 420 |
| GTCGGACACC AGTCTTACCC CCATAAGATC CTTACGGGCA GGCGCGAGAA GATGTATACC | 480 |
| ATTAGACAAA CAAATGGTCT CTCCGGATTC ACGAAGAGGT CGGAGTCCGA ATACGACTGC | 540 |
| TTTGGGACTG GTCACTCTTC AACCACAATC TCCGCAGGAC TCGGAATGGC AGTGGGAAGG | 600 |
| GACCTGAAGG GCAAGAAGAA CAATGTTGTG GCAGTCATTG GGGATGGTGC CATGACCGCT | 660 |
| GGACAGGCGT ACGAGGCCAT GAACAACGCC GGCTATCTTG ACTCGGATAT GATCGTTATT | 720 |
| TTGAACGACA ATAAGCAAGT GTCCCTCCCT ACGGCTACTC TGGATGGACC AATCCCTCCA | 780 |
| GTGGGTGCCC TGTCGTCCGC TTTGTCCCGC CTCCAGAGCA ACCGGCCACT GAGAGAGCTT | 840 |
| CGCGAAGTTG CAAAGGGCGT GACCAAGCAA ATCGGTGGAC CGATGCACGA GTGGGCCGCT | 900 |
| AAGGTGGACG AATACGCCCG GGGGATGATT AGCGGATCTG GCTCAACACT CTTCGAGGAA | 960 |
| CTTGGTTTGT ACTATATCGG ACCTGTCGAT GGCCATAATA TTGACGATTT GATCGCTATT | 1020 |
| CTCAAGGAGG TGAAGTCCAC CAAGACGACT GGCCCAGTCC TGATCCACGT CGTTACAGAG | 1080 |
| AAGGGGCGCG GTTACCCGTA TGCGGAAAAG GCGGCAGACA AGTACCATGG CGTCACGAAG | 1140 |
| TTCGATCCGG CGACTGGGAA GCAGTTTAAG GGTTCGGCAA TCACCCAATC CTACACCACA | 1200 |
| TATTTCGCCG AGGCTCTCAT TGCGGAGGCA GAAGTCGACA AGGATATCGT TGCCATTCAC | 1260 |
| GCAGCTATGG GAGGAGGCAC CGGCCTCAAC CTGTTCCTTC GGAGATTTCC TACAAGATGC | 1320 |
| TTCGACGTCG GCATCGCGGA GCAGCATGCA GTTACATTTG CGGCAGGACT TGCCTGCGAA | 1380 |
| GGCTTGAAGC CCTTCTGTGC TATCTACAGC TCTTTTATGC AGAGGGCGTA TGATCAAGTG | 1440 |
| GTCCACGACG TGGATTTGCA GAAGCTCCCA GTCCGCTTCG CCATGGACAG AGCTGGACTC | 1500 |
| GTGGGAGCAG ATGGTCCAAC GCATTGCGGA GCCTTCGACG TCACTTTTAT GGCTTGTCTC | 1560 |
| CCAAACATGG TTGTGATGGC CCCGTCAGAT GAGGCTGAAC TGTTCCACAT GGTGGCTACC | 1620 |
| GCAGCTGCAA TCGACGATAG ACCATCCTGT TTTCGCTACC CGAGAGGAAA CGGCGTCGGA | 1680 |
| GTTCAGCTGC CACCGGGAAA TAAGGGCATT CCGCTCGAGG TCGGCAAGGG ACGCATCCTG | 1740 |
| ATTGAGGGCG AACGGGTTGC GCTCCTGGGC TATGGGACGG CAGTGCAGAG CTGCCTCGCA | 1800 |
| GCAGCTTCTC TGGTCGAGCC TCATGGCCTT TTGATCACGG TTGCCGACGC TCGCTTCTGT | 1860 |
| AAGCCCCTGG ATCACACTCT TATTCGGTCT TTGGCCAAGT CACATGAGGT CCTCATCACT | 1920 |
| GTTGAGGAAG GATCAATTGG AGGCTTCGGC TCGCACGTGG CGCACTTCCT CGCACTCGAC | 1980 |
| GGGCTCCTGG ATGGCAAGCT CAAGTGGAGA CCTCTGGTTC TTCCCGACAG GTACATCGAT | 2040 |
| CACGGGTCGC CATCCGTGCA GCTTATTGAG GCTGGTTTGA CCCCGAGCCA TGTGGCGGCA | 2100 |
| ACAGTCCTGA ACATCCTTGG CAATAAGAGG GAAGCGCTGC AAATTATGTC ATCGTGAGGT | 2160 |
| ACCTCTAGAA AGCTT | 2175 |
| Farnesyl pyrophosphate synthase (farnesyl disphosphate synthase) | |
| (5 examples; SEQ ID NO: 29 is based on | |
| Saccharomyces cerevisiae polypeptide sequence) | |
| (with chloroplast targeting sequence) | |

SEQ ID NO: 21

| | |
|---|---|
| GGATCCGAGC TCATGGCACC GACAGTTATG GCATCATCCG CTACAGCCGT TGCTCCTTTC | 60 |
| CAGGGGTTGA AGTCCACCGC TACTCTTCCC GTTGCGAGGA GGTCCACCAC CTCCTTCGCG | 120 |
| AAGGTGTCAA ACGGCGGGAG GATCAGGTGC ATGGCATCGG AGAAGGAAAT TAGGCGCGAG | 180 |
| CGCTTCCTGA ACGTCTTTCC TAAGCTGGTT GAGGAACTTA ATGCCTCGCT CCTGGCTTAC | 240 |
| GGCATGCCCA AGGAGGCCTG TGACTGGTAC GCTCACTCCC TCAACTATAA TACGCCAGGT | 300 |
| GGAAAGTTGA ACAGGGGGCT CAGCGTGGTC GATACGTACG CCATCCTGTC TAATAAGACT | 360 |
| GTCGAGCAGC TTGGTCAAGA GGAATATGAA AAGGTTGCTA TCTTGGGATG GTGCATTGAG | 420 |
| CTTTTGCAGG CGTACTTCCT GGTCGCAGAC GATATGATGG ACAAGTCCAT CACCCGGAGA | 480 |
| GGCCAACCAT GTTGGTATAA GGTTCCGGAA GTGGGGGAAA TCGCGATTAA CGACGCATTC | 540 |
| ATGCTGGAGG CCGCTATCTA CAAGCTCCTG AAGTCACACT TTCGCAACGA GAAGTACTAT | 600 |
| ATCGACATTA CGGAGCTGTT CCATGAAGTT ACGTTTCAGA CTGAGCTGGG CCAACTGATG | 660 |
| GATCTTATCA CTGCGCCCGA AGACAAGGTG GATCTGTCTA AGTTCTCACT TAAGAAGCAC | 720 |
| TCCTTCATTG TCACCTTTAA GACAGCCTAC TATAGCTTTT ACCTGCCTGT GGCGCTTGCA | 780 |
| ATGTATGTCG CCGGCATCAC AGACGAGAAG GATCTTAAGC AGGCTCGGGA CGTGTTGATC | 840 |
| CCGCTCGGCG AGTACTTCCA GATTCAAGAC GATTATCTCG ATTGCTTTGG AACCCCTGAG | 900 |
| CAGATCGGCA AGATTGGGAC AGACATCCAA GATAACAAGT GTTCTTGGGT TATTAATAAG | 960 |
| GCCCTTGAGT TGGCCTCAGC TGAACAGAGA AAGACCCTGG ACGAGAACTA CGGCAAGAAG | 1020 |
| GATAGCGTGG CGGAAGCAAA GTGCAAGAAG ATTTTCAACG ACTTGAAGAT TGAGCAGCTC | 1080 |
| TACCATGAAT ATGAGGAATC TATCGCCAAG GATCTCAAGG CTAAGATTTC GCAAGTCGAC | 1140 |
| GAGTCCCGGG GCTTCAAGGC GGATGTTTTG ACAGCATTTC TCAATAAGGT GTACAAGAGA | 1200 |
| TCCAAGTGAG GTACCTCTAG AAAGCTT | 1227 |

SEQ ID NO: 22

| | |
|---|---|
| GGATCCGAGC TCATGGCTGA TCTGAAGTCG ACGTTTTTGA AGGTGTATTC CGTTCTGAAG | 60 |
| CAGGAGTTGC TGGAGGACCC CGCATTTGAG TGGACCCCTG ACTCCAGGCA GTGGGTCGAG | 120 |
| CGCATGCTCG ATTACAACGT TCCTGGCGGG AAGCTCAATC GGGGCCTGTC TGTGATTGAC | 180 |
| TCATATAAGC TCCTGAAGGA GGGGCAAGAA CTTACCGAGG AAGAGATTTT CCTCGCGTCC | 240 |
| GCATTGGGTT GGTGCATTGA GTGGTTGCAG GCCTACTTTC TCGTCCTGGA CGATATCATG | 300 |
| GACTCCAGCC ACACAAGGCG CGGCCAACCT TGTTGGTTCA GGGTGCCCAA GGTCGGACTG | 360 |
| ATCGCAGCTA ACGATGGGAT TCTTTTGCGG AATCACATCC CCCGCATCCT CAAGAAGCAT | 420 |
| TTTCGCGGCA AGGCTTACTA TGTTGACCTC CTGGATTTGT TCAACGAAGT GGAGTTTCAG | 480 |
| ACCGCGTCTG GTCAAATGAT CGACCTCATT ACCACACTGG AAGGAGAGAA GGATCTCTCG | 540 |
| AAGTACACCC TTTCCTTGCA CCGGAGAATC GTCCAGTACA AGACAGCATA CTATAGCTTC | 600 |
| TATCTGCCAG TTGCCTGCGC TCTTTTGATT GCCGGCGAGA ACCTCGACAA TCATATCGTG | 660 |
| GTCAAGGATA TTCTGGTGCA GATGGGTATC TACTTCCAGG TCCAAGACGA TTATCTCGAC | 720 |
| TGTTTTGGAG ATCCGGAGAC GATCGGCAAG ATCGGAACTG ACATCGAAGA TTTCAAGTGC | 780 |
| TCCTGGCTCG TTGTGAAGGC ACTCGAGCTG TGTAACGAGG AGCAGAAGAA GGTGCTGTAC | 840 |
| GAACACTATG GCAAGGCCGA CCCAGCAAGC GTCGCCAAGG TCAAGGTTCT TTACAACGAG | 900 |
| CTTAAGTTGC AAGGGGTTTT CACGGAATAC GAGAACGAGT CATATAAGAA GCTGGTCACT | 960 |
| AGCATCGAGG CTCATCCATC TAAGCCGGTT CAGGCTGTGC TTAAGTCGTT TTTGGCGAAG | 1020 |
| ATATACAAGA GGCAAAAGTG AGGTACCTCT AGAAAGCTT | 1059 |
| (with chloroplast targeting sequence) | |

SEQ ID NO: 23

| | |
|---|---|
| GGATCCGAGCTCATGGCACCAACCGTCATGGCATCGTCCGCAACCGCCGTCGCACCTTTC | 60 |
| CAGGGTCTGAAGTCAACAGCAACACTCCCAGTCGCAAGAAGGTCTACCACATCATTCGCA | 120 |
| AAGGTGTCCAACGGCGGGAGGATCAGGTGCATGGCCGACCTTAAGTCCACGTTCTTGAAG | 180 |
| GTGTACAGCGTCCTCAAGCAGGAGCTGCTCGAGGACCCAGCTTTTGAGTGGACTCCCGAT | 240 |
| TCACGGCAATGGGTGGAAAGAATGCTGGACTACAACGTCCCAGGTGGCAAGCTCAATCGC | 300 |
| GGTTTGTCCGTGATCGATTCCTACAAGCTCTTGAAGGAGGGACAGGAACTTACCGAGGAA | 360 |
| GAGATTTTCCTCGCGTCCGCACTGGGCTGGTGCATTGAGTGGTTGCAGGCCTACTTTCTT | 420 |
| GTCTTGGACGATATCATGGACTCCAGCCACACAAGGCGCGGGCAACCATGTTGGTTCCGG | 480 |
| GTTCCGAAAGTGGGTCTCATCGCCGCTAACGATGGCATCCTCCTGAGGAATCACATCCCG | 540 |
| CGCATTCTTAAGAAGCATTTTAGAGGCAAGGCATACTATGTCGACCTTTTGGATTTGTTC | 600 |
| AACGAAGTTGAGTTTCAGACGGCCAGCGGCCAAATGATCGACCTTATTACGACTTTGGAA | 660 |
| GGGGAGAAGGATCTTAGCAAGTACACGCTCTCTCTGCACCGGAGAATCGTGCAGTACAAG | 720 |
| ACTGCTTACTATTCTTTCTATCTGCCTGTCGCCTGCGCTCTCCTGATTGCGGGCGAGAAC | 780 |
| CTCGACAATCATATCGTGGTCAAGGATATTCTGGTTCAGATGGGCATCTACTTCCAGGTG | 840 |
| CAAGACGATTATCTGGACTGTTTTGGCGACCCAGAGACCATCGGCAAGATTGGGACAGAC | 900 |
| ATCGAAGATTTCAAGTGCTCGTGGCTCGTTGTGAAGGCTCTTGAGTTGTGTAACGAGGAG | 960 |
| CAGAAGAAGGTTCTGTACGAGCACTATGGCAAGGCGGACCCAGCATCCGTCGCCAAGGTC | 1020 |
| AAGGTTCTCTACAACGAGCTGAAGCTGCAAGGAGTGTTCACCGAATACGAGAACGAGTCT | 1080 |
| TATAAGAAGCTGGTCACATCAATCGAGGCGCATCCATCGAAGCCGGTCCAGGCTGTTCTC | 1140 |
| AAGTCATTTCTGGCGAAGATATACAAGCGGCAAAAGTGAGGTACCTCTAGAAAGCTT | 1197 |

SEQ ID NO: 24

| | |
|---|---|
| GGATCCGAGC TCATGGCGTC AGAGAAGGAG ATTAGAAGGG AGAGGTTTTT GAATGTTTTC | 60 |
| CCCAAGCTGG TTGAAGAGTT GAATGCGTCA CTGCTGGCAT ACGGTATGCC TAAGGAGGCG | 120 |
| TGCGACTGGT ACGCACACTC CCTGAACTAT AATACCCCCG GCGGGAAGTT GAACCGGGGA | 180 |
| CTCTCGGTGG TCGATACCTA CGCCATCCTG TCCAATAAGA CAGTTGAGCA GCTTGGCCAA | 240 |
| GAGGAATATG AAAAGGTGGC TATCTTGGGG TGGTGCATTG AGCTGCTGCA GGCCTACTTC | 300 |
| CTCGTTGCTG ACGATATGAT GGACAAGTCT ATCACAAGGC GCGGTCAACC ATGTTGGTAT | 360 |
| AAGGTTCCGG AAGTGGGAGA AATCGCCATT AACGACGCTT TCATGCTGGA GGCCGCTATC | 420 |
| TACAAGCTCT TGAAGAGCCA CTTTCGCAAC GAGAAGTACT ATATCGACAT TACCGAGCTG | 480 |
| TTCCATGAAG TCACCTTTCA GACAGAGCTT GGTCAATTGA TGGATCTCAT CACAGCCCCT | 540 |
| GAAGACAAGG TCGATCTGTC CAAGTTCAGC CTTAAGAAGC ACAGCTTCAT TGTTACGTTT | 600 |
| AAGACTGCGT ACTATTCTTT CTACCTGCCG GTCGCGCTTG CAATGTATGT TGCGGGCATC | 660 |
| ACGGACGAGA AGGATCTGAA GCAGGCAAGG GACGTGCTGA TCCCACTTGG CGAGTACTTC | 720 |
| CAGATTCAAG ACGATTATCT TGATTGCTTT GGGACGCCGG AGCAGATCGG CAAGATCGGA | 780 |
| ACTGACATCC AAGATAACAA GTGTTCATGG GTCATCAACA AGGCCCTCGA GCTGGCATCG | 840 |
| GCTGAACAGC GCAAGACGCT GGACGAGAAC TACGGCAAGA AGGATTCCGT CGCGGAAGCA | 900 |
| AAGTGCAAGA AGATTTTCAA CGACTTGAAG ATTGAGCAGC TCTACCATGA ATATGAGGAA | 960 |
| AGCATCGCGA AGGATCTCAA GGCAAAGATT TCTCAAGTCG ACGAGTCACG GGGGTTCAAG | 1020 |
| GCCGATGTGT TGACTGCTTT TCTCAACAAG GTCTACAAGA GATCCAAGTA AGGTACCAAG | 1080 |
| CTT | 1083 |

SEQ ID NO: 29

| | |
|---|---|
| ATGGCGTCAG AGAAGGAGAT TAGAAGGGAG AGGTTTTTGA ATGTTTTCCC CAAGCTGGTT | 60 |
| GAAGAGTTGA ATGCGTCACT GCTGGCATAC GGTATGCCTA AGGAGGCGTG CGACTGGTAC | 120 |
| GCACACTCCC TGAACTATAA TACCCCCGGC GGGAAGTTGA ACCGGGGACT CTCGGTGGTC | 180 |
| GATACCTACG CCATCCTGTC CAATAAGACA GTTGAGCAGC TTGGCCAAGA GGAATATGAA | 240 |
| AAGGTGGCTA TCTTGGGGTG GTGCATTGAG CTGCTGCAGG CCTACTTCCT CGTTGCTGAC | 300 |
| GATATGATGG ACAAGTCTAT CACAAGGCGC GGTCAACCAT GTTGGTATAA GGTTCCGGAA | 360 |
| GTGGGAGAAA TCGCCATTAA CGACGCTTTC ATGCTGGAGG CCGCTATCTA CAAGCTCTTG | 420 |
| AAGAGCCACT TTCGCAACGA GAAGTACTAT ATCGACATTA CCGAGCTGTT CCATGAAGTC | 480 |
| ACCTTTCAGA CAGAGCTTGG TCAATTGATG GATCTCATCA CAGCCCCTGA AGACAAGGTC | 540 |
| GATCTGTCCA AGTTCAGCCT TAAGAAGCAC AGCTTCATTG TTACGTTTAA GACTGCGTAC | 600 |
| TATTCTTTCT ACCTGCCGGT CGCGCTTGCA ATGTATGTTG CGGGCATCAC GGACGAGAAG | 660 |
| GATCTGAAGC AGGCAAGGGA CGTGCTGATC CCACTTGGCG AGTACTTCCA GATTCAAGAC | 720 |
| GATTATCTTG ATTGCTTTGG GACGCCGGAG CAGATCGGCA AGATCGGAAC TGACATCCAA | 780 |
| GATAACAAGT GTTCATGGGT CATCAACAAG GCCCTCGAGC TGGCATCGGC TGAACAGCGC | 840 |
| AAGACGCTGG ACGAGAACTA CGGCAAGAAG GATTCCGTCG CGGAAGCAAA GTGCAAGAAG | 900 |
| ATTTTCAACG ACTTGAAGAT TGAGCAGCTC TACCATGAAT ATGAGGAAAG CATCGCGAAG | 960 |
| GATCTCAAGG CAAAGATTTC TCAAGTCGAC GAGTCACGGG GGTTCAAGGC CGATGTGTTG | 1020 |
| ACTGCTTTTC TCAACAAGGT CTACAAGAGA TCCAAGTAA | 1059 |
| β-farnesene synthase (two examples) | |
| (with chloroplast targeting sequence) | |

SEQ ID NO: 25

| | |
|---|---|
| GGATCCGAGC TCATGGCCCC TACGGTCATG GCGTCCTCAG CGACTGCGGT TGCACCCTTT | 60 |
| CAAGGTCTCA AGAGCACGGC GACACTCCCT GTGGCACGGA GATCGACCAC ATCCTTCGCC | 120 |
| AAGGTTTCCA ACGGCGGGAG AATCAGGTGC ATGGACACGC TGCCAATTTC CAGCGTCTCA | 180 |
| TTTTCTTCAT CGACTTCGCC TCTTGTGGTC GACGATAAGG TTTCGACGAA GCCCGACGTG | 240 |
| ATCAGGCACA CTATGAACTT CAATGCTTCA ATTTGGGGCG ATCAGTTTCT GACCTACGAC | 300 |
| GAGCCAGAGG ACCTCGTGAT GAAGAAGCAA CTCGTTGAGG AACTGAAGGA GGAAGTGAAG | 360 |
| AAGGAGCTGA TCACAATTAA GGGTAGCAAT GAGCCGATGC AGCACGTGAA GCTCATCGAG | 420 |
| TTGATTGACG CGGTCCAACG CTTGGGAATC GCATACCATT TCGAGGAAGA GATCGAAGAG | 480 |
| GCCCTTCAGC ACATTCATGT CACCTACGGC GAGCAGTGGG TTGATAAGGA AAACTTGCAA | 540 |
| TCAATTTCGC TCTGGTTCCG CCTCCTGCGG CAGCAAGGTT TTAATGTGTC CAGCGGAGTC | 600 |
| TTCAAGGACT TTATGGATGA GAAGGGCAAG TTCAAGGAAT CTCTCTGCAA CGACGCGCAG | 660 |
| GGAATCCTTG CATTGTACGA GGCCGCTTTC ATGCGGGTGG AGGACGAAAC CATTCTTGAT | 720 |
| AATGCGTTGG AGTTTACAAA GGTCCACTTG GATATCATTG CAAAGGACCC GTCATGTGAT | 780 |
| TCTTCACTCA GAACCCAGAT CCATCAAGCC CTCAAGCAGC CACTGAGGAG AAGACTTGCA | 840 |
| AGGATCGAGG CACTGCACTA CATGCCGATC TACCAGCAAG AGACATCCCA TGACGAAGTT | 900 |
| CTTTTGAAGC TCGCTAAGCT GGATTTCTCG GTGTTGCAGT CCATGCACAA GAAGGAGCTG | 960 |
| AGCCATATCT GCAAGTGGTG GAAGGACCTC GATCTGCAAA ACAAGCTGCC TTACGTGCGC | 1020 |
| GACCGGGTTG TGGAGGGCTA TTTCTGGATT CTCTCCATCT ACTATGAGCC CCAGCACGCG | 1080 |
| AGAACCAGGA TGTTTCTGAT GAAGACATGC ATGTGGCTTG TCGTTTTGGA CGATACGTTC | 1140 |
| GACAATTACG GTACTTATGA AGAGCTGGAG ATTTTCACCC AAGCAGTGGA ACGCTGGTCC | 1200 |
| ATTAGCTGTC TCGATATGCT GCCTGAGTAC ATGAAGCTCA TCTATCAGGA GCTTGTTAAC | 1260 |
| TTGCACGTGG AGATGGAGGA GAGCCTGGAG AAGGAAGGGA AGACGTACCA AATTCATTAT | 1320 |
| GTCAAGGAGA TGGCCAAGGA ACTGGTGAGA AATTACCTTG TCGAGGCTAG GTGGCTGAAG | 1380 |
| GAAGGCTACA TGCCCACCCT TGAAGAGTAT ATGTCTGTCT CAATGGTTAC GGGCACTTAC | 1440 |
| GGGCTCATGA TCGCGCGCTC TTATGTGGGT CGGGGAGACA TTGTCACCGA GGATACATTC | 1500 |
| AAGTGGGTCT CGTCCTACCC ACCGATCATT AAGGCGTCCT GCGTTATCGT GCGCCTGATG | 1560 |
| GACGATATTG TCAGCCACAA GGAAGAGCAG GAGCGGGGCC ATGTTGCAAG CTCTATCGAG | 1620 |
| TGCTACAGCA AGGAATCTGG GGCCTCCGAA GAGGAGGCCT GCGAGTATAT CTCTCGCAAG | 1680 |
| GTTGAAGACG CCTGGAAGGT CATCAACAGA GAGTCACTGA GGCCAACGGC TGTGCCTTTC | 1740 |
| CCCCTCCTGA TGCCGGCCAT CAACTTGGCT CGGATGTGTG AGGTCCTCTA CAGCGTTAAT | 1800 |
| GACGGCTTCA CTCACGCCGA GGGGGATATG AAGAGCTATA TGAAGTCTTT CTTTGTCCAT | 1860 |
| CCTATGGTGG TCTGAGGTAC CTCTAGAAAG CTT | 1893 |

SEQ ID NO: 26

| | |
|---|---|
| GGATCCGAGC TCATGGATAC CCTGCCTATT TCGTCCGTCT CGTTCTCCTC TTCTACGTCG | 60 |
| CCACTGGTCG TCGATGATAA GGTGTCTACA AAGCCTGATG TGATCCGCCA CACGATGAAC | 120 |
| TTCAATGCCT CTATCTGGGG CGACCAGTTT CTGACTTACG ACGAGCCTGA GGACCTCGTG | 180 |
| ATGAAGAAGC AACTCGTCGA GGAACTGAAG GAAGAAGTCA AGAAGGAGCT GATCACGATT | 240 |
| AAGGGCTCAA ACGAGCCCAT GCAGCACGTG AAGCTCATCG AGTTGATTGA CGCGGTGCAA | 300 |
| AGGCTGGGGA TCGCATACCA TTTCGAGGAA GAGATCGAAG AGGCTCTTCA GCACATTCAT | 360 |
| GTGACATACG GCGAGCAGTG GGTCGATAAG GAAAACTTGC AATCAATTTC GCTCTGGTTC | 420 |
| AGACTCCTGA GGCAGCAAGG CTTTAATGTC TCCAGCGGGG TTTTCAAGGA CTTTATGGAT | 480 |
| GAGAAGGGCA AGTTCAAGGA ATCGCTCTGC AACGACGCGC AGGGCATCCT CGCATTGTAC | 540 |
| GAGGCCGCTT TCATGCGCGT TGAGGACGAA ACCATTCTTG ATAATGCGTT GGAGTTTACA | 600 |
| AAGGTCCACT TGGATATCAT TGCAAAGGAC CCTTCTTGTG ATTCTTCACT CCGCACGCAG | 660 |
| ATCCATCAAG CCCTCAAGCA GCCTCTGAGG AGAAGACTTG CAAGAATCGA GGCACTGCAC | 720 |
| TACATGCCCA TCTACCAGCA AGAGACTTCC CATGACGAAG TCCTTTTGAA GCTCGCTAAG | 780 |
| CTGGATTTCT CTGTTTTGCA GTCAATGCAC AAGAAGGAGC TGAGCCATAT CTGCAAGTGG | 840 |
| TGGAAGGACC TCGATCTGCA AAACAAGTTG CCATACGTGA GAGACAGGGT GGTCGAGGGG | 900 |
| TATTTCTGGA TTCTCTCCAT CTACTATGAG CCGCAGCACG CGCGCACGCG GATGTTTCTG | 960 |
| ATGAAGACTT GCATGTGGCT TGTTGTGTTG GACGATACCT TCGACAATTA CGGCACATAT | 1020 |
| GAAGAGCTGG AGATTTTCAC CCAAGCAGTG GAAAGGTGGT CCATTAGCTG TCTCGATATG | 1080 |
| CTGCCAGAGT ACATGAAGCT CATCTATCAG GAGCTTGTGA ACTTGCACGT CGAGATGGAG | 1140 |
| GAGAGCCTGG AGAAGGAAGG AAAGACCTAC CAAATTCATT ATGTCAAGGA GATGGCCAAG | 1200 |
| GAACTGGTCC GCAATTACCT TGTTGAGGCT CGGTGGCTGA AGGAAGGCTA CATGCCGACA | 1260 |
| CTTGAAGAGT ATATGTCTGT TTCAATGGTG ACCGGTACAT ACGGACTCAT GATCGCCAGA | 1320 |
| TCCTATGTTG GCAGGGGGGA CATTGTGACG GAGGATACTT TCAAGTGGGT GTCGTCCTAC | 1380 |
| CCACCGATCA TTAAGGCGAG CTGCGTGATC GTCAGACTGA TGGACGATAT TGTGTCTCAC | 1440 |
| AAGGAAGAGC AGGAGAGGGG TCATGTCGCA AGCTCTATCG AGTGCTACTC GAAGGAATCC | 1500 |
| GGAGCCAGCG AAGAGGAGGC CTGCGAGTAT ATCTCAAGAA AGGTCGAAGA TGCCTGGAAG | 1560 |
| GTTATTAATA GAGAGTCGCT GAGACCAACC GCTGTGCCTT TCCCACTCCT GATGCCGGCC | 1620 |
| ATCAACTTGG CTCGGATGTG TGAGGTTCTC TACAGCGTGA ATGACGGTTT TACACACGCC | 1680 |
| GAGGGAGATA TGAAGTCGTA TATGAAGTCC TTCTTTGTCC ATCCAATGGT CGTTTAAGGT | 1740 |
| ACCAAGCTT | 1749 |
| OVP1 | |

SEQ ID NO: 27

| | |
|---|---|
| GGATCCGAGC TCATGAATCC TTCCGCAAGA ATTTCGCAAG TGGCAATGGC AGCAATCCTC | 60 |
| CCCGATCTGG CTACGCAGGT GTTGGTTCCC GCCGCAGCGG TGGTCGGCAT CGCTTTCGCG | 120 |
| GTTGTGCAGT GGGTGCTGGT CTCTAAGGTC AAGATGACGG CAGAGAGGAG AGGAGGAGAA | 180 |
| GGATCTCCTG GAGCAGCTGC AGGCAAGGAC GGTGGAGCAG CCTCAGAGTA CCTTATCGAG | 240 |
| GAAGAGGAAG GGTTGAACGA ACACAATGTC GTTGAGAAGT GCTCCGAAAT CCAGCATGCG | 300 |
| ATTTCGGAGG GCGCAACCTC CTTCCTCTTT ACAGAATACA AGTATGTGGG GCTTTTTATG | 360 |
| GGTATCTTCG CCGTCTTGAT CTTCCTCTTC CTCGGATCTG TTGAGGGCTT CTCTACCAAG | 420 |
| TCACAACCTT GCCACTACTC AAAGGATAGG ATGTGTAAGC CCGCACTTGC CAACGCTATC | 480 |
| TTTAGCACCG TTGCCTTCGT GTTGGGCGCT GTGACATCGC TTGTCTCCGG GTTCTTGGGT | 540 |
| ATGAAGATCG CCACCTATGC GAATGCAAGA ACCACACTGG AGGCTAGGAA GGGAGTCGGC | 600 |
| AAGGCGTTTA TTACAGCATT CAGAAGCGGG GCCGTGATGG GTTTCCTCCT GGCTGCGTCT | 660 |
| GGCCTCGTGG TCCTGTACAT CGCTATTAAC CTCTTTGGAA TCTACTATGG CGACGATTGG | 720 |
| GAGGGCCTGT TCGAAGCCAT TACGGGATAC GGTCTCGGAG GGTCCAGCAT GGCTCTGTTC | 780 |
| GGTAGGGTTG GTGGAGGCAT CTATACTAAG GCAGCCGACG TGGGTGCTGA TCTCGTCGGA | 840 |
| AAGGTTGAGC GCAACATTCC AGAAGACGAT CCTCGGAATC CCGCCGTGAT CGCAGACAAC | 900 |
| GTTGGGGATA ATGTGGGTGA CATTGCGGGA ATGGGCAGCG ACCTTTTCGG CTCTTACGCG | 960 |
| GAGTCTTCAT GCGCTGCGTT GGTTGTGGCA TCCATCTCGT CCTTTGGCAT TAATCATGAG | 1020 |
| TTCACCCCAA TGCTGTATCC GCTTTTGATT AGCTCTGTCG GGATCATTGC GTGTCTTATC | 1080 |
| ACGACTTTGT TCGCAACTGA CTTCTTTGAG ATCAAGGCCG TGGATGAGAT TGAACCTGCT | 1140 |
| CTCAAGAAGC AGCTGATCAT TAGCACGGTC GTTATGACTG TGGGCATCGC GCTCGTCTCT | 1200 |
| TGGCTCGGGC TGCCCTACTC ATTCACGATT TTCAACTTTG GCGCCCAGAA GACTGTCTAT | 1260 |
| AATTGGCAAC TCTTCCTCTG CGTTGCGGTG GGACTTTGGG CAGGCTTGAT CATTGGGTTC | 1320 |
| GTGACCGAGT ACTATACATC CAACGCCTAC AGCCCAGTGC AAGACGTCGC TGATAGCTGT | 1380 |
| CGCACGGGCG CAGCCACTAA TGTCATCTTT GGTCTCGCCC TGGGATATAA GTCAGTTATC | 1440 |
| ATTCCGATCT TCGCCATTGC TTTCTCGATC TTTCTCTCAT TCTCGCTGGC TGCGATGTAC | 1500 |
| GGCGTCGCGG TTGCAGCCCT TGGGATGTTG TCCACCATCG CAACAGGTCT GGCCATTGAC | 1560 |
| GCTTATGGAC CAATCTCGGA TAACGCCGGG GGTATTGCGG AGATGGCCGG TATGAGCCAC | 1620 |
| AGGATCAGGG AACGGACCGA CGCGCTTGAT GCTGCGGGAA ATACCACAGC AGCCATTGGG | 1680 |
| AAGGGTTTCG CAATCGGTTC AGCTGCGCTG GTGTCGCTTG CCTTGTTTGG AGCTTTCGTC | 1740 |
| TCCAGAGCAG CAATCAGCAC GGTGGACGTC CTCACTCCAA AGGTTTTTAT CGGCCTCATT | 1800 |
| GTGGGGGCGA TGCTGCCGTA CTGGTTCTCC GCAATGACCA TGAAGAGCGT CGGCTCTGCT | 1860 |
| GCGCTCAAGA TGGTTGAGGA AGTGCGGAGA CAGTTCAACA GCATCCCAGG TCTGATGGAG | 1920 |
| GGAACGACTA AGCCGGACTA CGCCACCTGC GTCAAGATTT CTACAGATGC TTCAATCAAG | 1980 |
| GAGATGATTC CACCAGGCGC CCTCGTGATG CTGTCCCCAC TTATCGTCGG CATTTTCTTT | 2040 |
| GGGGTTGAGA CACTCTCGGG TCTCCTGGCA GGAGCACTGG TCTCCGGCGT TCAAATCGCC | 2100 |
| ATTTCCGCTA GCAACACCGG AGGCGCGTGG GACAATGCAA AGAAGTACAT CGAGGCAGGA | 2160 |
| GCTTCCGAAC ACGCACGCAC ACTGGGACCT AAGGGCAGCG ATTGTCATAA GGCAGCCGTG | 2220 |
| ATCGGCGATA CGATTGGGGA CCCTCTCAAG GATACTTCAG GCCCCTCGTT GAACATCCTC | 2280 |
| ATTAAGCTGA TGGCTGTCGA GTCCCTGGTT TTCGCCCCCT TCTTTGCTAC CCATGGGGGT | 2340 |
| ATCCTTTTTA AGTGGTTCTA AGGTACCAAG CTT | 2373 |

Preferably, the plant has a large reserve of carbon-rich energy-storage molecules, in the form of sucrose (such as sweet sorghum and sugarcane) or resin (such as guayule), which are readily available for diversion into the production of β-farnesene.

The invention, in some embodiments, modifies guayule as a biofuel crop by increasing the expression of genes coding for proteins catalyzing the rate-limiting steps of β-farnesene synthesis, resulting in production and accumulation of high-energy, β-farnesene-rich, terpenoid resins in guayule's native specialized resin vessel cells. Guayule naturally produces up to 28% hydrocarbon on a dry weight basis (polyisoprene-rubber and resin)(Tipton and Gregg, 1982).

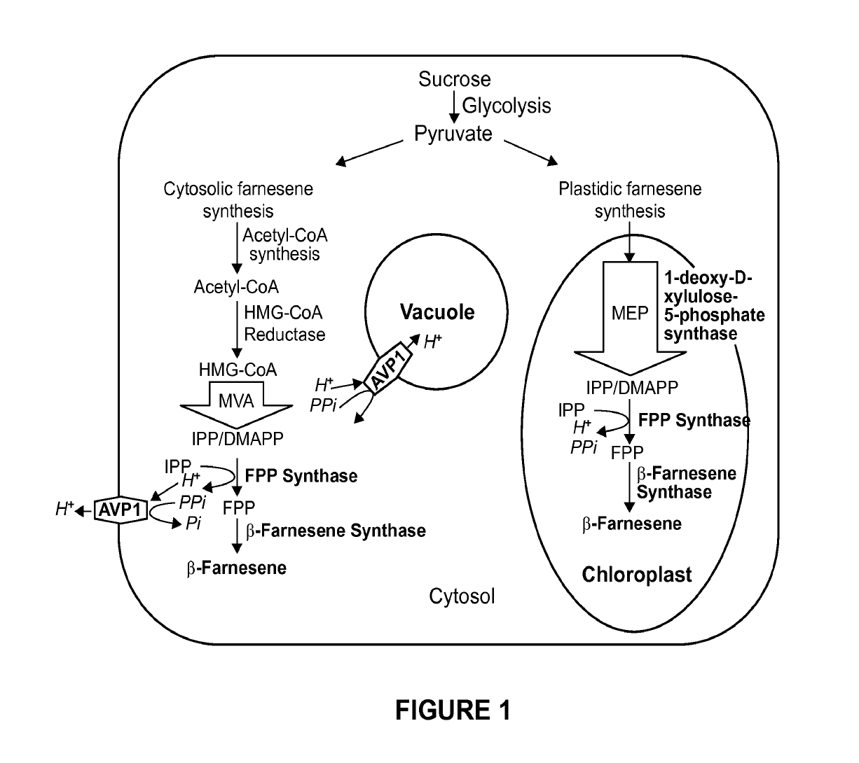

In both guayule and sorghum, as in many other plants, terpenoid synthesis occurs through the cytosolic mevalonic acid pathway (MVA) and the methylerythritol phosphate pathway (MEP), the latter of which is localized to the plastidic compartment (FIG. 1)(Cheng et al., 2007). In some embodiments of the invention, increasing the expression of rate-limiting proteins routes the already large carbon reserves destined in some resin-rich, stored carbon-rich, and stored sugar-rich plants, such as guayule to resin and rubber, and in sorghum to stored sucrose, into the formation of β-farnesene. In these embodiments, the sum total of carbon flux through photosynthesis into the formation of sucrose and downstream secondary metabolites remain unchanged, with alterations in carbon flux occurring only in pathways involved in secondary metabolites (i.e. terpenoids). As these fluxes can be difficult to quantify using standard metabolic labeling/flux analysis techniques, such diversion of carbon can be quantified through the terpenoid synthesis pathways by (1) assaying the expression levels and activities of enzymes up-regulated the modified plants or plant cells, (2) determining the amounts of terpenoid resin and precursors (IPP, FPP) using accelerated solvent extraction (discussed below), and (3) quantifying amounts, and species as desired, of the produced secondary compounds, including HMG-CoA, methylerythritol phosphate, GPP, FPP, β-farnesene, and any other sesquiterpenoid moieties through LC/MS. By fully defining and quantifying all of the intermediates involved in the pathways being engineered, this approach will allow us to both determine the relative carbon flux in our transgenic lines, as well as identify any potential bottlenecks that would result in accumulation of “upstream” precursors. Near Infra-red Spectroscopy (NIR) models can be developed to allow high through put screening of high farnesene transgenics (Cornish, 2004).

In some embodiments, β-farnesene synthesis in the cytosol is engineered to be up-regulated. These embodiments take advantage of the fact that the enzymes encoding terpenoid synthesis up to farnesene pyrophosphate are already present and functional in this cellular compartment. In cytosolic terpenoid synthesis, pyruvate formed from the glycolysis of sucrose molecules is converted into Acetyl-CoA which is itself incorporated into hydroxymethylglutaryl-coenzyme A (HMG-CoA) by the enzyme HMG-CoA reductase (Bach et al., 1991; Enjuto et al., 1994). As HMG-CoA reductase catalyzes the rate-limiting step in sesquiterpenoid production in the cytosol, this gene is over-expressed to funnel carbon from photosynthate into terpenoid production. HMG-CoA involved in terpenoid synthesis is then processed through the MVA pathway and used to generate dimethylallyl pyrophosphate (DMAPP) and isopentenyl pyrophosphate (IPP), both 5-carbon isoprene monomers for terpenoid biosynthesis (Bach et al., 1991; Cheng et al., 2007; Enjuto et al., 1994). These monomers are assembled together in a series of head-to-tail condensation reactions to generate farnesyl pyrophosphate (FPP, C15), a reaction catalyzed by the enzyme farnesyl pyrophosphate synthase (FPP synthase/FPPS). To specifically direct the increased partitioning of carbon resulting from elevation of HMG-CoA synthesis into production of C15 sesquiterpenoids, expression of FPPS is increased in some embodiments (Cunillera et al., 1996). As shown in FIG. 1, the condensation reactions catalyzed by geranyl diphosphate synthase (GPPS) and FPPS also result in the formation of both pyrophosphate and a free proton as byproducts which, if allowed to accumulate, result in acidification of the cytosol. To prevent this, in some embodiments, vacuolar pyrophosphatases, such as AVP1 (Li et al., 2005), and the rice ortholog, OVP1 (Sakakibara, 1996) are over-expressed; in some embodiments, OVP1 and AVP1 are specifically expressed in tissues where GPPS and FPPS expression have been increased. Under normal conditions, AVP1 functions by using the energy generated by pyrophosphate hydrolysis to transport protons into the vacuole (Li et al., 2005). Over-expression of AVP1 in Arabidopsis leads to an increase in proton transport, as well as transport of protons into the apoplastic space by both ectopically expressed AVP1 and the plasma-membrane ATPase, which showed increased activation/plasma membrane localization following AVP1 over-expression (Li et al., 2005). Increased expression of AVP1 also increased plant resistance to both water stress in both Arabidopsis and cotton, an additional benefit (Gaxiola, 2001).

Simultaneously up-regulating the expression of the enzymes catalyzing rate-limiting steps in FPP and β-farnesene synthesis result in a dramatically increased pool of cytosolic FPP available for conversion into β-farnesene. This final reaction is catalyzed by the enzyme β-farnesene synthase, which in some embodiments, is also overexpressed; and in additional embodiments, in conjunction with terpenoid synthases and AVP1/OVP1 transporters. Many characterized sesquiterpene synthases exhibit some degree of promiscuity, i.e. they are able to accept multiple isoprenoid substrates and/or produce multiple products from FPP (Schnee et al., 2006) (Tholl, 2006). To ensure that β-farnesene is the predominant product produced by the modified plant cells and plants of the invention, β-farnesene synthase gene, preferably from a plant other than the plant or plant cell being modified, is introduced, or the endogenous β-farnesene synthase gene up-regulated. This gene has been demonstrated to function in both monocot (maize) and dicot (Arabidopsis) systems, and to produce primarily β-farnesene (as well as α-bergamotene, β-sesquiphellandrene, β-bisabolene, α-zingiberene, and sesquisabinene in lesser amounts) (Schnee et al., 2006). These sesquiterpenoid molecules exhibit hydrocarbon structures (and therefore energetic yields) almost identical to those of β-farnesene as shown in Table 1 and discussed previously.

In alternative embodiments, β-farnesene synthesis is up-regulated in the non-photosynthetic pro-plastids of stem cortical tissues. In previous studies, sugarcane (a monocot closely related to sorghum) pro-plastids have successfully produced and stored the secondary compound polyhydroxybutyric acid (a bioplastic) (Petrasovits, 2007), thus in some embodiments of the invention, β-farnesene can be stored in this cellular compartment. Plastidic IPP synthesis occurs via the MEP pathway (FIG. 1) (Cheng et al., 2007; Estevez et al., 2000). In this pathway, pyruvate from the glycolysis of sucrose in the cytosol is imported into the plastid and funneled through the MEP pathway to generate the IPP/DMAPP 5-carbon isoprene building blocks of polyterpenoid molecules. GPP synthase enzymes then use these precursors to make C-10 geranyl pyrophosphate. Unlike the cytosol, however, no FPP synthase enzyme is present in the plastid and, instead, two GPP molecules are linked together to form the diterpene geranylgeranyl pyrophosphate (GGPP, C20). In some embodiment, to ensure that terpenoid accumulation remains confined to the plastid and limit putative toxic effects, all cytosol-expressed proteins (except HMG-CoA reductase) are routed to this subcellular compartment by adding an N-terminal signal sequence targeting them to the chloroplast (Bohlmann, 1998; Van den Broeck, 1985; von Heijne, 1989; Wienk, 2000). Thus is some embodiments where the engineered plant cell or plant produces β-farnesene in the plastid, a similar strategy to engineering β-farnesene cytosolic synthesis, except in such emobdiments, the AVP1 is not targeted to the plastids. In further embodiments, the 1-deoxy-D-xylulose-5-phosphate synthase (DXS), which is the rate limited step in the MEP pathway limiting the production of IPP, is expressed in the nucleus (in lieu of the HMG-CoA reductase involved in cytosolic terpenoid production) and targeted to the plastids (Estevez et al., 2000).

As both metabolic engineering approaches used to drive β-farnesene production may result in a substantial drain on cellular metabolism, as well as impose the risk of reduced cell growth or cell death, targeting the genetic manipulations described in the various embodiments of the invention to specific cells and tissues can provide vigorous modified plant cells and plants. For example, guayule produces and stores large quantities of terpenoid resin in specialized resin vessel cells. Global expression of genes involved in terpenoid synthesis results in increased terpenoid accumulation in the resin vessels (Veatch et al., 2005). Therefore, in some embodiments directed to guayule and similar species, the enzymes catalyzing β-farnesene synthesis are also expressed globally in all plant tissues—resulting in the accumulation of β-farnesene-rich resin in resin vessels or such other compartment. Alternatively, some embodiments localize gene expression to resin vessel cells using, for example, resin vessel-specific promoters or other control elements.

In species, like sorghum, that do not possess specialized resin storage cells, tissue localization of β-farnesene synthesis can be preferable in some embodiments to generate a high farnesene sorghum plant cell or plant. In some embodiments, the transgenes encoding the enzymes of β-farnesene synthesis are operably linked to a global promoter, such as the PEPC promoter. Under these conditions, β-farnesene accumulates in part in all tissues. In alternative embodiments, β-farnesene production is targeted to mature stem cells involved in actively recruiting carbon-rich photosynthate to maximize production and minimize possible toxic effects. To ensure that the targeted internode regions have enough sucrose or other carbon source available for substantial β-farnesene production, those plant cells and plants producing large stores of carbon, such as high-sucrose sorghum lines, are preferably used. In such embodiments, the β-farnesene synthesis genes are driven by promoters involved in secondary cell wall synthesis (Bell-Lelong et al., 1997; Liang et al., 1989; Maury et al., 1999; Nair et al., 2002) (for example, sorghum cinnamate 4-hydroxylase, coumarate 3-hydroxylase, and caffeic acid O-methyl transferase). At 30-40% of the stem internode mass these cells represent a considerable storage volume. In lemon grass, an analogous system, limonene is stored in similar cells with secondary cell walls (LEWINSOHN et al., 1998). In some embodiments, especially in those instances where such an approach results in funneling of carbon away from cell wall production and reducing plant structural integrity, β-farnesene production can be localized to another plant compartment, such as the ground tissue cortical cells of sorghum internodes; this is accomplished by operably-linking the transgenese to promoters specific to the plant compartment. Such promoters are readily identified by those of skill in the art. For example, in sweet sorghum, the internode ground tissue cortical cells make up the majority of the internode mass (50-60%) and are involved in sucrose storage, so that a ready supply of carbon flux is available. In some embodiments, global and tissue-specific transgenes are used in the same plant cell or plant; these embodiments can be produced either by introducing all such transgenes into one host plant, or combined through crossing transgenic plants using conventional techniques.

In yet further embodiments, especially in those plant cells and plants that do not have a sufficient endogenous store of carbon to support an increase overall carbon incorporation/flux to produce β-farnesene at high levels, carbon capture enhancement can be applied. This technology can also improve carbon capture in plant cells and plants that have sufficient carbon stores to significantly produce β-farnesene, such as sweet sorghum and guayule. Carbon capture enhancement (CCE) technology approaches can increase the amount of carbon available to metabolically engineered β-farnesene pathways. For example, some mutations in the FVE gene results in significant increases in leaf chlorophyll, numbers of stem and guard cell chloroplasts, and >50% overall increase in total carbon incorporation into photosynthate. Plant cells and plants can be transformed with carbon capture enhancement constructs (such as GWD or FVE).

### Alternative Embodiments for Modulating β-Farnesene Synthase

Table 1 shows alternative genes that can be used to produce the modified plant cells and plants of the invention. In addition β-farnesene synthase isoforms with increased substrate specificity can be engineered for increased substrate using rational engineering of the active site, which has been demonstrated for other terpene synthases (Greenhagen et al., 2006; Yoshikuni and University of California, 2007). Such engineering focuses on β-farnesene synthases previously isolated and characterized from maize and wild teosinte relatives (Kollner et al., 2009). Simultaneously, β-farnesene synthases from other plant species, including Artemisia annua (Picaud S, 2005), Japanese citrus (Maruyama T, 2001), mint (Crock J, 1997), and Douglas fir (Huber D P, 2005), are expressed in multiple expression systems (including E. coli and yeast) and characterize. Such expressed proteins are modeled against known sesquiterpene synthase three-dimensional structures, and residues in and around the active site are identified and altered, generating specificity variants which are screened for improved performance.

Alternative Carbon Capture Technology:

A second CCE gene, GWD, when selectively silenced in cereal endosperm, is thought to significantly increase vegetative growth rates throughout the growing period, resulting in an approximate 20% increase in carbon capture through an unknown mode of action. Plants can be separately transformed with GWD. Since the FVE and GWD technologies work independently, CCE may increase the total carbon capture by 20% or more through the individual or combined effects of GWD, FVE or both. By using this carbon capture technology in conjunction with over-expression of terpenoid synthesis genes the increased flux of carbon generated by CCE is routed into the synthesis of terpenoid resins. Plants can be transformed separately with farnesene metabolic engineering (FME) MCs and CCE Agrobacterium constructs, and the respective transgenic lines crossed to integrate the two technologies.

Chloroplast Transformation.

In some embodiments, instead of using signal peptides to target nuclear-encoded enzymes to pro-plastids, genes involved in β-farnesene synthesis are introduced directly into the chloroplast genome of the target plant cell or plant. In such embodiments, IPP levels are increased by transforming with MEV genes cassette, and include FPPS and β-farnesene synthase. These embodiments are especially attractive when the chloroplast genome is known, such as in guayule (Kumar, 2009), or otherwise suitable insertion sites have been identified to engineer the chloroplast genome.

Genetic Transformation—Mini-Chromosomes, Transformation Techniques, Quantification of Farnesene

### A. Selected Embodiments

In some embodiments, mini-chromosomes, or other large DNA constructs that is used to introduce large numbers of genes simultaneously into the genome of a plant cell or plan, are exploited to express the multiple genes involved in β-farnesene production and proton-pyrophosphatases. A main advantage of using min-chromosomes, which are autonomously maintained by plant cells, is that the expression of genes carried on mini-chromosomes is not affected by position effects commonly observed in traditional engineered crops. Large gene payloads and stable expression are ideal for pathway engineering projects, and require fewer transgenic lines to be screened for commercial applications.

One aspect of the invention is related to plants containing functional, stable, autonomous MCs, preferably carrying one or more exogenous nucleic acids, such as FME gene stacks. Such plants carrying MCs are contrasted to transgenic plants with genomes that have been altered by chromosomal integration of an exogenous nucleic acid. Expression of the exogenous nucleic acid results in an altered phenotype of the plant. The invention provides for MCs comprising at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 250, 500, 1000 or more exogenous nucleic acids.

Any plant, including bryophytes, algae, seedless vascular plants, monocots, dicots, gymnosperm, field crops, vegetable crops, fruit and vine crops, can be modified by carrying autonomous MCs. Plant parts or plant tissues, including pollen, silk, endosperm, ovule, seed, embryo, pods, roots, cuttings, tubers, stems, stalks, fiber (lint), square, boll, fruit, berries, nuts, flowers, leaves, bark, epidermis, vascular tissue, whole plant, plant cell, plant organ, protoplast, crown, callus culture, petiole, petal, sepal, stamen, stigma, style, bud, meristem, cambium, cortex, pith, sheath, cell culture, or any group of plant cells organized into a structural and functional unit, any cells of can carry MCs.

A related aspect of the invention is plant parts or plant tissues, including pollen, silk, endosperm, ovule, seed, embryo, pods, roots, cuttings, tubers, stems, stalks, crown, fiber (lint), square, boll, callus culture, petiole, petal, sepal, stamen, stigma, style, bud, fruit, berries, nuts, flowers, leaves, bark, wood, whole plant, plant cell, plant organ, protoplast, cell culture, or any group of plant cells organized into a structural and functional unit comprising the nucleic acid constructs of the invention, whether maintained autonomously or integrated into the host plant cell chromosomes. In one preferred embodiment, the exogenous nucleic acid is primarily expressed in a specific location or tissue of a plant, for example, epidermis, fiber (lint), boll, square, vascular tissue, meristem, cambium, cortex, pith, leaf, sheath, flower, root or seed. Tissue-specific expression can be accomplished with, for example, localized presence of the MC, selective maintenance of the MC, or with promoters that drive tissue-specific expression.

Another related aspect of the invention is meiocytes, pollen, ovules, endosperm, seed, somatic embryos, apomyctic embryos, embryos derived from fertilization, vegetative propagules and progeny of the originally min-chromosome-containing plant and of its filial generations that retain the functional, stable, autonomous MC. Such progeny include clonally propagated plants, embryos and plant parts as well as filial progeny from self- and cross-breeding, and from apomyxis.

The MC can be transmitted to subsequent generations of viable daughter cells during mitotic cell division with a transmission efficiency of at least 1%, 5%, 10%, 20%, 30%, 40%, 50%, 60%, 70%, 80%, 85%, 90%, 95%, 96%, 97%, 98%, or 99%. The MC is transmitted to viable gametes during meiotic cell division with a transmission efficiency of at least 1%, 5%, 10%, 20%, 30%, 40%, 50%, 60%, 70%, 80%, 85%, 90%, 95%, 96%, 97%, 98%, or 99% when more than one copy of the MC is present in the gamete mother cells of the plant. The MC is transmitted to viable gametes during meiotic cell division with a transmission frequency of at least 1%, 5%, 10%, 20%, 30%, 40%, 45%, 46%, 47%, 48%, or 49% when one copy of the MC is present in the gamete mother cells of the plant and meiosis produces four viable products (e.g. typical male meiosis) When meiosis produces fewer than four viable products (e.g. typical female meiosis) a phenomenon called meiotic drive can cause the preferential segregation of particular chromosomes into the viable product resulting in higher than expected transmission frequencies of monoosmes through meiosis including at least 51%, 60%, 70%, 80%, 90% 95%, 96%, 97%, 98%, or 99%. For production of seeds via sexual reproduction or by apomyxis, the MC can be transferred into at least 1%, 5%, 10%, 20%, 25%, 30%, 40%, 50%, 60%, 70%, 80%, 85%, 90%, 95%, 96%, 97%, 98%, or 99% of viable embryos when cells of the plant contain more than one copy of the MC. For sexual seed production or apomyxitic seed production from plants with one MC per cell, the MC can be transferred into at least 1%, 5%, 10%, 20%, 30%, 40%, 50%, 60%, 70%, 71%, 72%, 73%, 74%, 75% of viable embryos.

A MC that comprises an exogenous selectable trait or exogenous selectable marker can be used to increase the frequency in subsequent generations of min-chromosome-containing cells, tissues, gametes, embryos, endosperm, seeds, plants or progeny. For example, the frequency of transmission of MCs into viable cells, tissues, gametes, embryos, endosperm, seeds, plants or progeny can be significantly increased after mitosis or meiosis by applying a selection that favors the survival of min-chromosome-containing cells, tissues, gametes, embryos, endosperm, seeds, plants or progeny over cells, tissues, gametes, embryos, endosperm, seeds, plants or progeny lacking the MC.

Transmission efficiency can be measured as the percentage of progeny cells or plants that carry the MC by one of several assays, including detecting expression of a reporter gene (e.g., a gene encoding a fluorescent protein), PCR detection of a sequence that is carried by the MC, RT-PCR detection of a gene transcript for a gene carried on the MC, Western analysis of a protein produced by a gene carried on the MC, Southern analysis of the DNA (either in total or a portion thereof) carried by the MC, fluorescence in situ hybridization (FISH) or in situ localization by repressor binding. Efficient transmission as measured by some benchmark percentage indicates the degree to which the MC is stable through the mitotic and meiotic cycles. Plants of the invention can also contain chromosomally integrated exogenous nucleic acid in addition to the autonomous MCs. The min-chromosome-containing plants or plant parts, including plant tissues, can include plants that have chromosomal integration of some portion of the MC (e.g., exogenous nucleic acid or centromere sequence) in some or all cells of the plant. The plant, including plant tissue or plant cell, is still characterized as min-chromosome-containing, despite the occurrence of some chromosomal integration. A mini-chromosome-containing plant can also have a MC plus non-MC integrated DNA. For example, a standard integrated transgenic plant that subsequently has a MC delivered to it (by crossing or transformation) is a mini-chromosome-containing plant. Similarly, A mini-chromosome-containing plant that has an integrative transgene delivered to one or more of its chromosomes (including plastid or organellar chromosomes) remains a mini-chromosome-containing plant by virtue of the presence of the autonomous MC. In one aspect, the autonomous MC can be isolated from integrated exogenous nucleic acid by crossing the min-chromosome-containing plant containing the integrated exogenous nucleic acid with plants producing some gametes lacking the integrated exogenous nucleic acid and subsequently isolating offspring of the cross, or subsequent crosses, that are min-chromosome-containing but lack the integrated exogenous nucleic acid. This independent segregation of the MC is one measure of the autonomous nature of the MC.

Another aspect of the invention relates to methods for producing and isolating such min-chromosome-containing plants containing functional, stable, autonomous MCs carrying, for example, FME gene stacks.

In one embodiment, the invention contemplates improved methods for isolating native centromere sequences, such as those from guayule. In another embodiment, the invention contemplates methods for generating variants of native or artificial centromere sequences by passage through bacterial or plant or other host cells.

In yet another embodiment, the invention contemplates methods for co-delivery of growth-inducing genes with MCs that may also carry FME gene stacks. The growth delivery genes include Agrobacterium tumefaciens or Arhizogenes isopentenyl transferase (IPT) genes involved in cytokinin biosynthesis, plant IPT genes involved in cytokinin biosynthesis (from any plant), Agrobacterium tumefaciens IAAH, IAAM genes involved in auxin biosynthesis (indole-3-acetamide hydrolase and tryptophan-2-monooxygenase, respectively), Agrobacterium rhizogenes rolA, rolB and rolC genes involved in root formation, Agrobacterium tumefaciens Aux1, Aux2 genes involved in auxin biosynthesis (indole-3-acetamide hydrolase or tryptophan-2-monooxygenase genes), Arabidopsis thaliana leafy cotyledon genes (e.g., Lec1, Lec2) promoting embryogenesis and shoot formation, Arabidopsis thaliana ESR1 gene involved in shoot formation, Arabidopsis thaliana PGA6/WUSCHEL gene involved in embryogenesis (Zuo et al., 2002).

Another aspect of the invention relates to methods for using min-chromosome-containing plants containing a MC carrying an FME gene stack for producing chemical and fuel products by appropriate expression of exogenous FME nucleic acid(s) contained on a MC.

In some animal systems it has been possible to use MCs with centromeres from one species in the cells of a different species (Cavaliere et al., 2009). Thus, another aspect of the invention is a mini-chromosome-containing plant comprising a functional, stable, autonomous MC that contains centromere sequence derived from a different taxonomic plant species, or derived from a different taxonomic plant species, genus, family, order or class.

Yet another aspect of the invention provides novel autonomous MCs used to transform plant cells that are in turn used to generate a plant (or multiple plants). Exemplary MCs of the invention are contemplated to be of a size 2000 kb or less. Other exemplary sizes of MCs include less than or equal to, e.g., 1500 kb, 1000 kb, 900 kb, 800 kb, 700 kb, 600 kb, 500 kb, 450 kb, 400 kb, 350 kb, 300 kb, 250 kb, 200 kb, 150 kb, 100 kb, 90 kb, 80 kb, 70, kb, 60 kb, or 40 kb.

Novel centromere compositions as characterized by sequence content, size, spatial arrangement of sequence motifs, or other parameters. Exemplary sizes include a centromeric nucleic acid insert derived from a portion of plant genomic DNA, that is less than or equal to 1000 kb, 900 kb, 800 kb, 700 kb, 600 kb, 500 kb, 400 kb, 300 kb, 200 kb, 150 kb, 100 kb, 95 kb, 90 kb, 85 kb, 80 kb, 75 kb, 70 kb, 65 kb, 60 kb, 55 kb, 50 kb, 45 kb, 40 kb, 35 kb, 30 kb, 25 kb, 20 kb, 15 kb, 10 kb, 5 kb, 4 kb, 3 kb, 2 kb, or 1 kb.

The invention also contemplates MCs or other vectors comprising fragments or variants of the genomic DNA inserts of the described BAC clones, or naturally occurring descendants thereof, that retain the ability to segregate during mitotic or meiotic division, as well as min-chromosome-containing plants or parts containing these MCs. Other exemplary embodiments include fragments or variants of the genomic DNA inserts of any of the identified BAC clones, or descendants thereof, and fragments or variants of the centromeric nucleic acid inserts of any of the vectors or MCs identified herein.

In other exemplary embodiments, the invention contemplates MCs or other vectors comprising centromeric nucleotide sequence that when hybridized to 1, 2, 3, 4, 5, 6, 7, 8, 9, 10, 11, 12, 13, 14, 15, 16, 17, 18, 19, 20, 30, 40, 50, 60, 70, 80, 90, 100 or more probes, including those described in the Examples, under hybridization conditions described herein, e.g., low, medium or high stringency, provides relative hybridization scores as described in the Examples.

### B. Composition of MCS and MC Construction

The MC vector of the present invention can contain a variety of elements, including: (1) sequences that function as plant centromeres; (2) one or more exogenous nucleic acids; (3) sequences that function as an origin of replication, that can be included in the region that functions as plant centromere, and optional; (4) a bacterial plasmid backbone for propagation of the plasmid in bacteria, though this element may be designed to be removed prior to delivery to a plant cell; (5) sequences that function as plant telomeres (particularly if the MC is linear); (6) optionally, additional “stuffer DNA” sequences that serve to separate the various components on the MC from each other; (7) optionally, “buffer” sequences such as MARs or SARs; (8) optionally, marker sequences of any origin, including but not limited to plant and bacterial origin; (9) optionally, sequences that serve as recombination sites; and (10) optionally, “chromatin packaging sequences” such as cohesion and condensing binding sites.

### C. Centromere Compositions

The centromere in the MC of the present invention can comprise centromere sequences as known in the art, which have the ability to confer to a nucleic acid the ability to segregate to daughter cells during cell division. U.S. Pat. Nos. 6,649,347, 7,119, 250, 7,132,240 describe methods for identifying and isolating centromeres; U.S. Pat. Nos. 7,456,013, 7,235,716, 7,227,057, and 7,226,782 disclose corn, soy, Brassica and tomato centromeres respectively; U.S. Pat. Nos. 7,989,202 and 8,062,885 described crop plant centromere compositions generally; US Patent Application Publication Nos. U520100297769 and U520090222947 also describe corn centromere compositions, international patent application publication nos. WO2011011693, WO2011091332, and WO2011011685 describe sorghum, cotton and sugarcane centromeres, respectively, and internation patent application publication no. WO2009134814 describes some algae centromere compositions. Other centromere compositions are known in the art or can be identified using guidance from the aforementioned patents and patent applications.

For example, for guayule MC development, guayule genomic DNA from line AZ-2 can be isolated from etiolated seedlings. A Bacterial Artificial Chromosome (BAC) library is prepared in a modified pBeloBAC11 vector. The library is arrayed on nylon filters and hybridized with centromere-specific satellite or centromere-associated retrotransposon sequence probes. To identify probe sequences, guayule genomic DNA from line AZ-2 are sequenced. Centromere probes can then be amplified from genomic DNA, cloned and characterized, and FISH analysis, or other appropriate analysis technique used to confirm their centromere localization. For example, about 50 BAC clones obtained from library screening can be characterized at the molecular level and hybridized to guayule root tip metaphase chromosome spreads. The three BAC clones with highest content of centromere satellite repeats and retrotransposon sequences, and strongest and specific hybridization to centromere regions of metaphase chromosomes can be selected to build mini-chromosomes. To further ensure success, two forms of guayule can be transformed, such as the apomyctic hybrid line AZ-101 and a rapidly growing, facultative, apomictic epitype selected from AZ-2.

MC Sequence Content and Structure

Plant-expressed genes from non-plant sources can be modified to accommodate plant codon usage, to insert preferred motifs near the translation initiation ATG codon, to remove sequences recognized in plants as 5′ or 3′ splice sites, or to better reflect plant GC/AT content. Plant genes typically have a GC content of more than 35%, and coding sequences that are rich in A and T nucleotides can be problematic. For example, ATTTA motifs can destabilize mRNA; plant polyadenylation signals such as AATAAA at inappropriate positions within the message can cause premature truncation of transcription; and monocotyledons can recognize AT-rich sequences as splice sites.

Each exogenous nucleic acid or plant-expressed gene can include a promoter, a coding region and a terminator sequence, that can be separated from each other by restriction endonucleasc sites or recombination sites or both. Genes can also include introns, that can be present in any number and at any position within the transcribed portion of the gene, including the 5′ untranslated sequence, the coding region and the 3′ untranslated sequence. Introns can be natural plant introns derived from any plant, or artificial introns based on the splice site consensus that has been defined for plant species. Some intron sequences have been shown to enhance expression in plants. Optionally the exogenous nucleic acid can include a plant transcriptional terminator, non-translated leader sequences derived from viruses that enhance expression, a minimal promoter, or a signal sequence controlling the targeting of gene products to plant compartments or organelles.

The coding regions of the genes can encode any protein, including visible marker genes (for example, fluorescent protein genes, other genes conferring a visible phenotype), other screenable or selectable marker genes (for example, conferring resistance to antibiotics, herbicides or other toxic compounds, or encoding a protein that confers a growth advantage to the cell expressing the protein) or genes that confer some commercial or agronomic value to the min-chromosome-containing plant. Multiple genes can be placed on the same MC vector. The genes can be separated from each other by restriction endonuclease sites, homing endonuclease sites, recombination sites or any combinations thereof. Any number of genes can be present. Genes on a MC can be in any orientation with respect to one another and with respect to the other elements of the MC (e.g. the centromere).

The MC vector can also contain a bacterial plasmid backbone for propagation of the plasmid in bacteria such as E. coli, A. tumefaciens, or A. rhizogenes. The plasmid backbone can be that of a low-copy vector or mid to high level copy backbone. This backbone can contain the replicon of the F′ plasmid of E. coli. However, other plasmid replicons, such as the bacteriophage P1 replicon, or other low-copy plasmid systems, such as the RK2 replication origin, can also be used. The backbone can include one or several antibiotic-resistance genes conferring resistance to a specific antibiotic to the bacterial cell in that the plasmid is present. Examples of bacterial antibiotic-resistance genes include kanamycin-, ampicillin-, chloramphenicol-, streptomycin-, spectinomycin-, tetracycline- and gentamycin-resistance genes. The backbone can also be designed so that it can be excised from the MC prior to delivery to a plant cell. The use of flanking restriction enzyme sites or flanking site-specific recombination sites are both useful for constructing a removable backbone.

The MC vector can also contain plant telomeres. An exemplary telomere sequence is tttaggg (SEQ ID NO:16) or its complement. Telomeres stabilize the ends of linear chromosomes and facilitate the complete replication of the extreme termini of the DNA molecule.

Additionally, the MC vector can contain “stuffer DNA” sequences that serve to separate the various components on the MC. Stuffer DNA can be of any origin, synthetic, prokaryotic or eukaryotic, and from any genome or species, plant, animal, microbe or organelle. Stuffer DNA can range from 10 bp, 20 bp, 30 bp, 40 bp, 50 bp, 60 bp, 70 bp, 80 bp, 90 bp, 100 bp, 150 bp, 200 bp, 300 bp, 400 bp 500 bp, 750 bp, 1000 bp, 2000 bp, 5000 bp, 10 kb, 20 kb, 50 kb, 75 kb, 1 Mb to 10 Mb in length and can be repetitive in sequence, with unit repeats from 10 bp to 1 Mb. Examples of repetitive sequences that can be used as stuffer DNAs include rDNA, satellite repeats, retroelements, transposons, pseudogenes, transcribed genes, microsatellites, tDNA genes, short sequence repeats and combinations thereof. Alternatively, stuffer DNA can consist of unique, non-repetitive DNA of any origin or sequence. The stuffer sequences can also include DNA with the ability to form boundary domains, such as scaffold attachment regions (SARs) or matrix attachment regions (MARs). Stuffer DNA can be entirely synthetic, composed of random sequence, having any base composition, or any A/T or G/C content.

In one embodiment of the invention, the MC has a circular structure without telomeres. In another embodiment, the MC has a circular structure with telomeres. In a third embodiment, the MC has a linear structure with telomeres. A “linear” structure can be generated by cutting a circular MC that contains telomeres with an endonuclease(s), that exposes the telomeres at the ends of the resultant linear nucleic acid molecule that contains all of the sequence contained in the original, closed construct. A variant of this strategy is to separate two telomere elements with an antibiotic-resistance gene that is also excised upon linearization. In a fourth embodiment of the invention, the telomeres could be placed in such a manner that the bacterial replicon, backbone sequences, antibiotic-resistance genes and any other sequences of bacterial origin and present for the purposes of propagation of the MC in bacteria, can be removed from the plant-expressed genes, the centromere, telomeres, and other sequences by cutting the structure with an endonuclease(s). When removing intervening sequences to expose telomere elements during linearization site-specific recombination systems can be used instead of endoculeases. These linearization techniques result in a MC from which much of, or preferably all, bacterial sequences have been removed. In this embodiment, bacterial sequence present between or among the plant-expressed genes or other MC sequences are excised prior to removal of the remaining bacterial sequences by cutting the MC with a homing endonuclease, and re-ligating the structure or by using site-specific recombination systems. Particularly useful endonucleases are those that are present only at the desired linearization site (unique), including homing endonuclease sites. Alternatively, the endonucleases and their sites can be replaced with any specific DNA cutting mechanism and its specific recognition site, such as a rare-cutting endonuclease or recombinase and its specific recognition site, as long as that site is present in the MC.

Various structural configurations of the MC elements are possible. A centromere can be placed on a MC either between genes or outside a cluster of genes next to a telomere. Stuffer DNAs can be combined with these configurations including stuffer sequences placed inside the telomeres, around the centromere between genes or any combination thereof. Thus, a large number of alternative MC structures are possible, depending on the relative placement of centromere DNA, genes, stuffer DNAs, bacterial sequences, telomeres, and other sequences. Such variations in architecture are possible both for linear and for circular MCs.

Exemplary Centromere Components

The centromere can contain n copies of a centromere repeated nucleotide sequence, wherein n is at least 2. In another embodiment, the centromere contains n copies of interdigitated repeats. An interdigitated repeat is a DNA sequence that consists of two distinct repetitive elements that combine to create a unique permutation. Potentially any number of repeat copies capable of physically being placed on the recombinant construct could be included on the construct, including about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 120, 140, 150, 200, 300, 400, 500, 750, 1,000, 1,500, 2,000, 3,000, 5,000, 7,500, 10,000, 20,000, 30,000, 40,000, 50,000, 60,000, 70,000, 80,000, 90,000 and about 100,000, including all ranges in-between such copy numbers. Moreover, the copies can vary from each other, such as is commonly observed in naturally occurring centromeres. The length of the repeat can vary, but will preferably range from about 20 bp to about 360 bp, from about 20 bp to about 250 bp, from about 50 bp to about 225 bp, from about 75 bp to about 210 bp, such as a 92 bp repeat and a 97 bp repeat, from about 100 bp to about 205 bp, from about 125 bp to about 200 bp, from about 150 bp to about 195 bp, from about 160 bp to about 190 and from about 170 bp to about 185 bp including about 180 bp. The length of the repeat can also be about 100 to 210 bp; such as 100, 194, and 210 bp. The length of the repeat can also include larger sequences, from about 300 bp to about 10 kb, from about 1 kb to 9 kb, from about 2 kb to about 8 kb, from about 3 kb to about 7 kb, from about 4 kb to about 8 kb, including, for example, 982 bp, 2836 bp, 5788 bp and 8308 bp.

Modification of Centromeres Isolated from Native Plant Genome

Modification and changes can be made in the centromeric DNA segments of the current invention and still obtain a functional molecule with desirable characteristics. The following is a discussion based upon changing the nucleic acids of a centromere to create an equivalent, or even an improved, second generation molecule.

Mutated centromeric sequences are contemplated to be useful for increasing the utility of the centromere. It is specifically contemplated that the function of the centromeres of the current invention can be based in part or in whole upon the secondary structure of the DNA sequences of the centromere, modification of the DNA with methyl groups or other adducts, and/or the proteins that interact with the centromere. By changing the DNA sequence of the centromere, one can alter the affinity of one or more centromere-associated protein(s) for the centromere and/or the secondary structure or modification of the centromeric sequences, thereby changing the activity of the centromere. Alternatively, changes can be made in the centromeres that do not affect the activity of the centromere. Changes in the centromeric sequences that reduce the size of the DNA segment needed to confer centromere activity are particularly useful, as are changes that increase the fidelity with that the centromere is transmitted during mitosis and meiosis.

Modification of Centromeres by Passage Through Bacteria, Plant or Other Hosts or Processes

MC DNA sequence can also be a derivative of the parental clone or centromere clone having substitutions, deletions, insertions, duplications and/or rearrangements of one or more nucleotides in the nucleic acid sequence. Such nucleotide mutations can occur individually or consecutively in stretches of 1, 2, 3, 4, 5, 6, 7, 8, 9, 10, 20, 40, 50, 60, 70, 80, 90, 100, 150, 200, 300, 400, 500, 800, 1000, 2000, 4000, 8000, 10000, 50000, 100000, and about 200000, including all ranges in-between. Variations of MCs can arise through passage of MCs through various hosts including virus, bacteria, yeast, plant or other prokaryotic or eukaryotic organism and can occur through passage of multiple hosts or individual host. Variations can also occur by replicating the MC in vitro. Variations can also be specifically engineered into the MC using standard molecular biology techniques.

### D. Exemplary Exogenous Nucleic Acids Including Plant-Expressed Genes and Regulatory Elements

Of particular interest in the present invention are exogenous nucleic acids that when introduced into plants alter the phenotype of the plant, a plant organ, plant tissue, or portion of the plant, such as those shown in Table 1. Such exogenous nucleic acids can be delivered on MCs; or alternatively, using methods described herein or in, for example, U.S. Pat. No. 7,993,913, delivered to MCs already in a plant cell.

### E. Exemplary Plant Promoters, Regulatory Sequences and Targeting Sequences

Constitutive Expression promoters: Exemplary constitutive expression promoters include the ubiquitin promoter, the CaMV 35S promoter (U.S. Pat. Nos. 5,858,742 and 5,322,938); and the actin promoter (e.g., rice—U.S. Pat. No. 5,641,876).

Inducible Expression promoters: Exemplary inducible expression promoters include the chemically regulatable tobacco PR-1 promoter (e.g., tobacco—U.S. Pat. No. 5,614,395; maize—U.S. Pat. No. 6,429,362). Various chemical regulators can be used to induce expression, including the benzothiadiazole, isonicotinic acid, and salicylic acid compounds disclosed in U.S. Pat. Nos. 5,523,311 and 5,614,395. Other promoters inducible by certain alcohols or ketones, such as ethanol, include the alcA gene promoter from Aspergillus nidulan. Glucocorticoid-mediated induction systems can also be used (Aoyama and Chua, 1997). Another class of useful promoters are water-deficit-inducible promoters, e.g., promoters that are derived from the 5′ regulatory region of genes identified as a heat shock protein 17.5 gene (HSP 17.5), an HVA22 gene (HVA22), and a cinnamic acid 4-hydroxylasc gene (CA4H) of Zea mays. Another water-deficit-inducible promoter is derived from the rob-17 promoter. U.S. Pat. No. 6,084,089 discloses cold inducible promoters, U.S. Pat. No. 6,294,714 discloses light inducible promoters, U.S. Pat. No. 6,140,078 discloses salt inducible promoters, U.S. Pat. No. 6,252,138 discloses pathogen inducible promoters, and U.S. Pat. No. 6,175,060 discloses phosphorus deficiency inducible promoters.

Wound-Inducible Promoters can Also be Used.

Tissue-Specific Promoters: Exemplary promoters that express genes only in certain tissues are useful. For example, root-specific expression can be attained using the promoter of the maize metallothionein-like (MTL) gene (U.S. Pat. No. 5,466,785). U.S. Pat. No. 5,837,848 discloses a root-specific promoter. Another exemplary promoter confers pith-preferred expression (maize trpA gene and promoter; WO 93/07278). Leaf-specific expression can be attained, for example, by using the promoter for a maize gene encoding phosphoenol carboxylase. Pollen-specific expression can be conferred by the promoter for the maize calcium-dependent protein kinase (CDPK) gene that is expressed in pollen cells (WO 93/07278). U.S. Pat. Appl. Pub. No. 20040016025 describes tissue-specific promoters. Pollen-specific expression can also be conferred by the tomato LAT52 pollen-specific promoter. U.S. Pat. No. 6,437,217 discloses a root-specific maize RS81 promoter, U.S. Pat. No. 6,426,446 discloses a root specific maize RS324 promoter, U.S. Pat. No. 6,232,526 discloses a constitutive maize A3 promoter, U.S. Pat. No. 6,177,611 that discloses constitutive maize promoters, U.S. Pat. No. 6,433,252 discloses a maize L3 oleosin promoter that are aleurone and seed coat-specific promoters, U.S. Pat. No. 6,429,357 discloses a constitutive rice actin 2 promoter and intron, U.S. patent application Pub. No. 20040216189 discloses an inducible constitutive leaf-specific maize chloroplast aldolase promoter. Other plant tissue specific promoters are disclosed in U.S. Pat. Nos. 7,754,946, 7,323,622, 7,253,276, 7,141,427, 7,816,506, and 7,973,217, and in US Patent Application Publication No. 20100011460.

Optionally a plant transcriptional terminator can be used in place of the plant-expressed gene native transcriptional terminator. Exemplary transcriptional terminators are those that are known to function in plants and include the CaMV 35S terminator, the tml terminator, the nopaline synthase terminator and the pea rbcS E9 terminator. These can be used in both monocotyledons and dicotyledons.

Various intron sequences have been shown to enhance expression. For example, the introns of the maize Adh1 gene can significantly enhance expression, especially intron 1 (Callis et al., 1987). The intron from the maize bronzel gene also enhances expression. Intron sequences have been routinely incorporated into plant transformation vectors, typically within the non-translated leader. U.S. Patent Application Publication 2002/0192813 discloses 5′, 3′ and intron elements useful in the design of effective plant expression vectors.

A number of non-translated leader sequences derived from viruses are also known to enhance expression, and these are particularly effective in dicotyledonous cells. Specifically, leader sequences from Tobacco Mosaic Virus (TMV, the “omega-sequence”), Maize Chlorotic Mottle Virus (MCMV), and Alfalfa Mosaic Virus (AMV) can enhance expression. Other leader sequences known and include: picornavirus leaders, for example, EMCV leader (Encephalomyocarditis 5′ noncoding region); potyvirus leaders, for example, TEV leader (Tobacco Etch Virus); MDMV leader (Maize Dwarf Mosaic Virus); human immunoglobulin heavy-chain binding protein (BiP) leader; untranslated leader from the coat protein mRNA of alfalfa mosaic virus (AMV RNA 4); tobacco mosaic virus leader (TMV); or Maize Chlorotic Mottle Virus leader (MCMV).

A minimal promoter can also be incorporated. Such a promoter has low background activity in plants when there is no transactivator present or when enhancer or response element binding sites are absent. An example is the Bzl minimal promoter, obtained from the bronzel gene of maize. A minimal promoter can also be created by use of a synthetic TATA element. The TATA element allows recognition of the promoter by RNA polymerase factors and confers a basal level of gene expression in the absence of activation.

Sequences controlling the targeting of gene products also can be included. For example, the targeting of gene products to the chloroplast is controlled by a signal sequence found at the amino terminal end of various proteins that is cleaved during chloroplast import to yield the mature protein. These signal sequences can be fused to heterologous gene products to import heterologous products into the chloroplast. DNA encoding for appropriate signal sequences can be isolated from the 5′ end of the cDNAs encoding the RUBISCO protein, the CAB protein, the EPSP synthasc enzyme, the GS2 protein or many other proteins that are known to be chloroplast localized. Other gene products are localized to other organelles, such as the mitochondrion and the peroxisome (e.g., (Unger et al., 1989)). Examples of sequences that target to such organelles are the nuclear-encoded ATPases or specific aspartate amino transferase isoforms for mitochondria. Amino terminal and carboxy-terminal sequences are responsible for targeting to the ER, the apoplast, and extracellular secretion from aleurone cells. Amino terminal sequences in conjunction with carboxy terminal sequences can target to the vacuole.

Another element that can be introduced is a matrix attachment region element (MAR), such as the chicken lysozyme A element that can be positioned around an expressible gene of interest to effect an increase in overall expression of the gene and diminish position dependent effects upon incorporation into the plant genome.

Use of Non-Plant Promoter Regions Isolated from Drosophila melanogaster and Saccharomyces cerevisiae to Express Genes in Plants

The promoter in the MC can be derived from plant or non-plant species. For example, the nucleotide sequence of the promoter is derived from non-plant species for the expression of genes in plant cells, such as dicotyledon plant cells, such as cotton. Non-plant promoters can be constitutive or inducible promoters derived from insects, e.g., Drosophila melanogaster, or from yeast, e.g., Succharomyces cerevisiae. These non-plant promoters can be operably linked to nucleic acid sequences encoding polypeptides or non-protein-expressing sequences including antisense RNA, miRNA, siRNA, and ribozymes, to form nucleic acid constructs, vectors, and host cells (prokaryotic or eukaryotic), comprising the promoters.

The present invention also relates to isolated promoter sequences and to constructs, vectors, or plant host cells comprising one or more of the promoters operably linked to a nucleic acid sequence encoding a polypeptide or non-protein expressing sequence.

In the methods of the present invention, the promoter can also be a mutant of the promoters having a substitution, deletion, and/or insertion of one or more nucleotides in a native nucleic acid sequence of that element.

The techniques used to isolate or clone a nucleic acid sequence comprising a promoter of interest are known in the art and include isolation from genomic DNA.

### F. Constructing MCS by Site-Specific Recombination

Plant MCs can be constructed using site-specific recombination sequences (for example those recognized by the bacteriophage P1 Cre recombinase, or the bacteriophage lambda integrase, or similar recombination enzymes). A compatible recombination site, or a pair of such sites, is present on both the centromere containing DNA clones and the donor DNA clones. Incubation of the donor clone and the centromere clone in the presence of the recombinase enzyme causes strand exchange to occur between the recombination sites in the two plasmids; the resulting MCs contain centromere sequences as well as MC vector sequences. The DNA molecules formed in such recombination reactions is introduced into E. coli, other bacteria, yeast or plant cells by common methods in the field including, heat shock, chemical transformation, electroporation, particle bombardment, whiskers, or other transformation methods followed by selection for marker genes, including chemical, enzymatic, or color markers present on either parental plasmid, allowing for the selection of transformants harboring MCs.

### G. Methods of Detecting and Characterizing MCS in Plant Cells or of Scoring MC Performance in Plant Cells

Identification of Candidate Centromere Fragments by Probing BAC Libraries

Methods for identifying centromere sequences have been previously described. In one example, centromeres are identified that are neither highly methylated nor comprising of tandem repeats. In this method, all available genomic nucleic acid sequences from an organism are assembled into low-stringency contigs. Those contigs having the largest assemblies (i.e., many sequences aligned, “deep read”) are then further examined. The pool of “largest” assemblies can be the top 1%, 2%, 3%, 4%, 5%, 6%, 7%, or 10% or more. This pool of contigs is then examined first for contigs containing tandem repeats using commonly available software. These contigs are eliminated from the pool. A consensus sequence determined for the remaining contigs with the deepest reads. Probes are designed and synthesized based on the consensus sequence, and used in an assay that allows for the detection of centromere sequences, such as fluorescence in situ hybridization (FISH) of mitotic or meiotic metaphase chromosomes. Of course, any suitable assay can be used. When using FISH, for example, a good candidate for a centromere sequence is a probe that labels every primary constriction of every chromosome (though genomes of allopolyploids may contain distinct sub-genomes with distinct centromeres). If desired, the candidate sequence can be further tested with other morphological or functional assays.

Methods for determining consensus sequence are well known in the art, e.g., U.S. Pat. App. Pub. No. 20030124561; (Hall et al., 2002). These methods, including DNA sequencing, assembly, and analysis, are well known and there are many possible variations known to those skilled in the art. Other alignment parameters can also be useful such as using more or less stringent definitions of consensus.

Non-Selective MC Mitotic Inheritance Assays

The following assays can distinguish autonomous events from integrated events.

Assay #1: Transient Assay

MCs are tested for their ability to become established as chromosomes and their ability to be inherited in mitotic cell divisions. MCs are delivered to plant cells. The cells used can be at various stages of growth. In this example, a population in that some cells were undergoing division can be used. The MC is then assessed over the course of several cell divisions, by tracking the presence of a screenable marker, e.g., a visible marker gene such as one encoding a fluorescent protein. Following initial delivery into many single cells and several cell divisions, single transformed cells divide to form clusters of MC-containing cells if the MC is inherited well. Other exemplary embodiments of this method include delivering MCs to other mitotic cell types, including roots and shoot meristems.

Assay #2: Non-Lineage Based Inheritance Assays on Modified Transformed Cells and Plants

MC inheritance is assessed on modified cell lines and plants by following the presence of the MC over the course of multiple cell divisions. An initial population of MC containing cells is assayed for the presence of the MC, by the presence of a marker gene, such as a gene encoding a fluorescent protein, a colored protein, a protein assayable by histochemical assay, or a gene affecting cell morphology. All nuclei are stained with a DNA-specific dye including but not limited to DAPI, Hoechst 33258, OliGreen, Giemsa YOYO, or TOTO, allowing a determination of the number of cells that do not contain the MC. After the initial determination of the percent of cells carrying the MC, the cells are allowed to divide over the course of several cell divisions. The number of cell divisions, n, is determined by an appropriate method, such as monitoring the change in total weight of cells, monitoring the change in volume of the cells, or directly counting cells in an aliquot of the culture. After a number of cell divisions, the population of cells is again assayed for the presence of the MC. The loss rate per generation is calculated by the equation (I):

Loss rate per generation=1−(F/1)^{1/n} (I)

The population of MC-containing cells can include suspension cells, callus, roots, leaves, meristems, flowers, or any other tissue of modified plants, or any other cell type containing a MC.

Assay #3: Lineage-Based Inheritance Assays on Modified Cells and Plants

MC inheritance is assessed on modified cell lines and plants by following the presence of the MC over the course of multiple cell divisions. In cell types that allow for tracking of cell lineage, such as root cell files, trichomes, and leaf stomata guard cells, MC loss per generation does not need to be determined statistically over a population, it can be discerned directly through successive cell divisions. In other manifestations of this method, cell lineage can be discerned from cell position, or methods including but not limited to the use of histological lineage tracing dyes, and the induction of genetic mosaics in dividing cells.

In one example, the two guard cells of the stomata are daughters of a single precursor cell. To assay MC inheritance in this cell type, the epidermis of the leaf of a plant containing a MC is examined for the presence of the MC by the presence of a marker gene, including one encoding a fluorescent protein, a colored protein, a protein assayable by histochemical assay, or a gene affecting cell morphology. The number of loss events in which one guard cell contains the MC (L) and the number of cell divisions in which both guard cells contain the MC (B) are counted. The loss rate per cell division is determined as L/(L+B). Other lineage-based cell types are assayed in similar fashion. Similar assays have been used in yeast.

Lineal MC inheritance can also be assessed by examining root files or clustered cells in callus over time. Changes in the percent of cells carrying the MC indicate the mitotic inheritance.

Assay #4: Inheritance Assays on Modified Cells and Plants in the Presence of Chromosome Loss Agents

Assays #1-3 can be done in the presence of chromosome loss agents (e.g., colchicine, colcemid, caffeine, etopocide, nocodazole, Oryzalin, and trifluran). It is likely that autonomous MCs are more susceptible to loss induced by chromosome loss agents; therefore, autonomous MCs show a lower rate of inheritance in the presence of chromosome loss agents. These methods have been used to study chromosome loss in fruit flies and yeast.

### H. Transformation of Plant Cells and Plant Regeneration

Various methods can be used to deliver DNA into plant cells. These include biological methods, such as Agrobacterium, E. coli, and viruses; physical methods, such as biolistic particle bombardment, nanocopiea device, the Stein beam gun, silicon carbide whiskers and microinjection; electrical methods, such as electroporation; and chemical methods, such as the use of polyethylene glycol and other compounds that stimulate DNA uptake into cells (Dunwell, 1999) and U.S. Pat. No. 5,464,765.

Agrobacterium-Mediated Delivery

Several Agrobacterium species mediate the transfer of “T-DNA” that can be genetically engineered to carry a desired piece of DNA into many plant species. Plasmids used for delivery contain the T-DNA flanking the nucleic acid to be inserted into the plant. The major events marking the process of T-DNA mediated pathogenesis are induction of virulence genes, processing and transfer of T-DNA.

There are three common methods to transform plant cells with Agrobacterium. The first method is co-cultivation of Agrobacterium with cultured isolated protoplasts. This method requires an established culture system that allows culturing protoplasts and plant regeneration from cultured protoplasts. The second method is transformation of cells or tissues with Agrobacterium. This method requires (a) that the plant cells or tissues can be modified by Agrobacterium and (b) that the modified cells or tissues can be induced to regenerate into whole plants. The third method is transformation of seeds, apices or meristems with Agrobacterium. This method requires exposure of the meristematic cells of these tissues to Agrobacterium and micropropagation of the shoots or plant organs arising from these meristematic cells.

Those of skill in the art are familiar with procedures for growth and suitable culture conditions for Agrobacterium, as well as subsequent inoculation procedures. Liquid or semi-solid culture media can be used. The density of the Agrobacterium culture used for inoculation and the ratio of Agrobacterium cells to explant can vary from one system to the next, as can media, growth procedures, timing and lighting conditions.

Transformation of dicotyledons using Agrobacterium has long been known in the art, and transformation of monocotyledons using Agrobacterium has also been described (WO 94/00977; U.S. Pat. No. 5,591,616; U520040244075).

A number of wild-type and disarmed strains of Agrobacterium tumefaciens and Agrobacterium rhizogenes harboring Ti or Ri plasmids can be used for gene transfer into plants. Preferably, the Agrobacterium hosts contain disarmed Ti and Ri plasmids that do not contain the oncogenes that cause tumorigenesis or rhizogenesis. Exemplary strains include Agrobacterium tumefaciens strain CSS, a nopaline-type strain that is used to mediate the transfer of DNA into a plant cell, octopine-type strains such as LBA4404 or succinamopine-type strains, e.g., EHA101 or EHA105.

The efficiency of transformation by Agrobacterium can be enhanced by using a number of methods known in the art. For example, the inclusion of a natural wound response molecule such as acetosyringone (AS) to the Agrobacterium culture can enhance transformation efficiency with Agrobacterium tumefaciens. Alternatively, transformation efficiency can be enhanced by wounding the target tissue to be modified or transformed. Wounding of plant tissue can be achieved, for example, by punching, maceration, bombardment with microprojectiles, etc.

In addition, transfer of a disarmed Ti plasmid without T-DNA and another vector with T-DNA containing the marker enzyme beta-glucuronidase can be accomplished into three different bacteria other than Agrobacteria which adds to the transformation vector arsenal.

Micro Projectile Bombardment Delivery

In this process, the desired nucleic acid is deposited on or in small dense particles, e.g., tungsten, platinum, or preferably 1 micron gold particles, that are then delivered at a high velocity into the plant tissue or plant cells using a specialized biolistics device, such as are available from Bio-Rad Laboratories (Hercules, Calif.). The advantage of this method is that no specialized sequences need to be present on the nucleic acid molecule to be delivered into plant cells.

For bombardment, cells in suspension are concentrated on filters or solid culture medium. Alternatively, immature embryos, seedling explants, or any plant tissue or target cells can be arranged on solid culture medium. The cells to be bombarded are positioned at an appropriate distance below the microprojectile stopping plate.

Various biolistics protocols have been described that differ in the type of particle or the manner in that DNA is coated onto the particle. Any technique for coating microprojectiles that allows for delivery of transforming DNA to the target cells can be used. For example, particles can be prepared by functionalizing the surface of a gold oxide particle by providing free amine groups. DNA, having a strong negative charge, binds to the functionalized particles.

Parameters such as the concentration of DNA used to coat microprojectiles can influence the recovery of transformants containing a single copy of the transgene. For example, a lower concentration of DNA may not necessarily change the efficiency of the transformation but can instead increase the proportion of single copy insertion events. Ranges of approximately 1 ng to approximately 10 pg, approximately 5 ng to 8 μg or approximately 20 ng, 50 ng, 100 ng, 200 ng, 500 ng, 1 pg, 2 μg, 5 μg, or 7 μg of transforming DNA can be used per each 1.0-2.0 mg of starting 1.0 micron gold particles.

Other physical and biological parameters can be varied, such as manipulation of the DNA/microprojectile precipitate, factors that affect the flight and velocity of the projectiles, manipulation of the cells before and immediately after bombardment (including osmotic state, tissue hydration and the subculture stage or cell cycle of the recipient cells), the orientation of an immature embryo or other target tissue relative to the particle trajectory, and also the nature of the transforming DNA, such as linearized DNA or intact supercoiled plasmids. Physical parameters such as DNA concentration, gap distance, flight distance, tissue distance, and helium pressure, can be optimized.

The particles delivered via biolistics can be “dry” or “wet.” In the “dry” method, the MC DNA-coated particles such as gold are applied onto a macrocarrier (such as a metal plate, or a carrier sheet made of a fragile material, such as mylar) and dried. The gas discharge then accelerates the macrocarrier into a stopping screen that halts the macrocarrier but allows the particles to pass through. The particles are accelerated at, and enter, the plant tissue arrayed below on growth media. The media surrports plant tissue growth and development and are suitable for plant transformation and regeneration. These tissue culture media can either be purchased as a commercial preparation, or custom prepared and modified. Examples of such media include Murashige and Skoog (MS), N6, Linsmaier and Skoog, Uchimiya and Murashige, Gamborg's B5 media, D medium, MCCown's Woody plant media, Nitsch and Nitsch, and Schenk and Hildebrandt. Those of skill in the art are aware that media and media supplements such as nutrients and growth regulators for use in transformation and regeneration and other culture conditions such as light intensity during incubation, pH, and incubation temperatures can be optimized.

Those of skill in the art can use, devise, and modify selective regimes, media, and growth conditions depending on the plant system and the selective agent. Typical selective agents include antibiotics, such as geneticin (G418), kanamycin, paromomycin; or other chemicals, such as glyphosate or other herbicides.

MC Delivery without Selection

The MC is delivered to plant cells or tissues, e.g., plant cells in suspension to obtain stably modified callus clones for inheritance assays. Suspension cells are maintained in a growth media, for example Murashige and Skoog (MS) liquid medium containing an auxin such as 2,4-dichlorophenoxyacetic acid (2,4-D). Cells are bombarded using a particle bombardment process and propagated in the same liquid medium to permit the growth of modified and unmodified cells. Portions of each bombardment are monitored for formation of fluorescent clusters, which are then isolated by micromanipulation and cultured on solid medium. Clones modified with the MC are expanded, and homogenous clones are used in inheritance assays, or assays measuring MC structure or autonomy.

MC Transformation with Selectable Marker Gene

MC-modified cells in bombarded calluses or explants can be isolated using a selectable marker gene. The bombarded tissues are transferred to a medium containing an appropriate selective agent. Tissues are transferred into selection between 0 and about 7 days or more after bombardment. Selection of MC-modified cells can be further monitored by tracking fluorescent marker genes or by the appearance of modified explants (modified cells on explants can be green under light in selection medium, while surrounding non-modified cells are weakly pigmented). In plants that develop through shoot organogenesis (e.g., Brassica, tomato or tobacco), the modified cells can form shoots directly, or alternatively, can be isolated and expanded for regeneration of multiple shoots transgenic for the MC. In plants that develop through embryogenesis (e.g., corn or soybean), additional culturing steps may be necessary to induce the modified cells to form an embryo and to regenerate in the appropriate media.

For selection to be effective, the plant cells or tissue need to be grown on selective medium containing the appropriate concentration of antibiotic or killing agent, and the cells need to be plated at a defined and constant density. The concentration of selective agent and cell density are generally chosen to cause complete growth inhibition of wild type plant tissue that does not express the selectable marker gene; but allowing cells containing the introduced DNA to grow and expand into min-chromosome-containing clones. This critical concentration of selective agent typically is the lowest concentration at that there is complete growth inhibition of wild type cells, at the cell density used in the experiments. However, in some cases, sub-killing concentrations of the selective agent can be equally or more effective for the isolation of plant cells containing MC DNA, especially in cases where the identification of such cells is assisted by a visible marker gene (e.g., fluorescent protein gene) present on the MC.

In some species (e.g., tobacco or tomato), a homogenous clone of modified cells can also arise spontaneously when bombarded cells are placed under the appropriate selection. An exemplary selective agent is the neomycin phosphotransferase II (Nptll) marker gene that confers resistance to the antibiotics kanamycin, G418 (geneticin) and paramomycin. In other species, or in certain plant tissues or when using particular selectable markers, homogeneous clones may not arise spontaneously under selection; in this case the clusters of modified cells can be manipulated to homogeneity using the visible marker genes present on the MCs as an indication of that cells contain MC DNA.

Regeneration of Min-Chromosome-Containing Plants from Explants to Mature, Rooted Plants

For plants that develop through shoot organogenesis (e.g., Brassica, tomato and tobacco), regeneration of a whole plant involves culturing of regenerable explant tissues taken from sterile organogenic callus tissue, seedlings or mature plants on a shoot regeneration medium for shoot organogenesis, and rooting of the regenerated shoots in a rooting medium to obtain intact whole plants with a fully developed root system.

For plant species, such cotton, corn and soybean, regeneration of a whole plant occurs via an embryogenic step that is not necessary for plant species where shoot organogenesis is efficient. In these plants, the explant tissue is cultured on an appropriate media for embryogenesis, and the embryo is cultured until shoots form. The regenerated shoots are cultured in a rooting medium to obtain intact whole plants with a fully developed root system.

Explants are obtained from any tissues of a plant suitable for regeneration. Exemplary tissues include hypocotyls, internodes, roots, cotyledons, petioles, cotyledonary petioles, leaves and peduncles, prepared from sterile seedlings or mature plants.

Explants are wounded (for example with a scalpel or razor blade) and cultured on a shoot regeneration medium (SRM) containing Murashige and Skoog (MS) medium as well as a cytokinin, e.g., 6-benzylaminopurinc (BA), and an auxin, e.g., a-naphthaleneacetic acid (NAA), and an anti-ethylene agent, e.g., silver nitrate (AgNO₃). For example, 2 mg/L of BA, 0.05 mg/L of NAA, and 2 mg/L of AgNO₃ can be added to MS medium for shoot organogenesis. The most efficient shoot regeneration is obtained from longitudinal sections of internode explants.

Shoots regenerated via organogenesis are rooted in a MS medium containing low concentrations of an auxin such as NAA.

To regenerate a whole plant with a MC, explants are pre-incubated for 1 to 7 days (or longer) on the shoot regeneration medium prior to bombardment with MC (see below). Following bombardment, explants are incubated on the same shoot regeneration medium for a recovery period up to 7 days (or longer), followed by selection for transformed shoots or clusters on the same medium but with a selective agent appropriate for a particular selectable marker gene (see below).

Method of Co-Delivering Growth Inducing Genes to Facilitate Isolation of Ad Chromosomal Plant Cell Clones

Another method used in the generation of cell clones containing MCs involves the co-delivery of DNA containing genes that are capable of activating growth of plant cells, or that promote the formation of a specific organ, embryo or plant structure that is capable of self-sustaining growth. In one embodiment, the recipient cell receives simultaneously the MC, and a separate DNA molecule encoding one or more growth promoting, organogenesis-promoting, embryo genesis-promoting or regeneration-promoting genes. Following DNA delivery, expression of the plant growth regulator genes stimulates the plant cells to divide, or to initiate differentiation into a specific organ, embryo, or other cell types or tissues capable of regeneration. Multiple plant growth regulator genes can be combined on the same molecule, or co-bombarded on separate molecules. Use of these genes can also be combined with application of plant growth regulator molecules into the medium used to culture the plant cells, or of precursors to such molecules that are converted to functional plant growth regulators by the plant cell's biosynthetic machinery, or by the genes delivered into the plant cell.

The co-bombardment strategy of MCs with separate DNA molecules encoding plant growth regulators transiently supplies the plant growth regulator genes for several generations of plant cells following DNA delivery. During this time, the MC can be stabilized by virtue of its centromere, but the DNA molecules encoding plant growth regulator genes, or organogenesis-promoting, embryogenesis-promoting or re generation-promoting genes tend to be lost. The transient expression of these genes, prior to their loss, can give the cells containing MC DNA a sufficient growth advantage, or sufficient tendency to develop into plant organs, embryos or a regenerable cell cluster, to outgrow the non-modified cells in their vicinity, or to form a readily identifiable structure that is not formed by non-modified cells. Loss of the DNA molecule encoding these genes prevents phenotypes from manifesting themselves that can be caused by these genes if present through the remainder of plant regeneration. In rare cases, the DNA molecules encoding plant growth regulator genes integrate into the host plant's genome or into the MC.

Alternatively, the genes promoting plant cell growth can be genes promoting shoot formation or embryogenesis, or giving rise to any identifiable organ, tissue or structure that can be regenerated into a plant. In this case, embryos or shoots harboring MCs directly after DNA delivery are obtained without the need to induce shoot formation with growth activators, or lowering the growth activator treatment necessary to regenerate plants. The advantages of this method are more rapid regeneration, higher transformation efficiency, lower background growth of non-modified tissue, and lower rates of morphologic abnormalities in the regenerated plants.

Determination of MC Structure and Autonomy in Min-Chromosome-Containing Plants and Tissues

The structure and autonomy of the MC in min-chromosome-containing plants and tissues can be determined by: conventional and pulsed-field Southern blot hybridization to genomic DNA from modified tissue subjected or not subjected to restriction endonuclease digestion, dot blot hybridization of genomic DNA from modified tissue hybridized with different MC specific sequences, MC rescue, exonuclease activity, PCR on DNA from modified tissues with probes specific to the MC, or FISH to nuclei of modified cells. Table 4 below summarizes these methods.

TABLE 4
Autonomous MC assays

| Assay | Details | Potential outcome | Interpretation |
|---|---|---|---|
| Southern blot | Restriction digest of genomic DNA compared to | 1. Native sizes and pattern of bands | 1. Autonomous or integrated via |
| | purified MC | | CEN fragment |
| | | 2. Altered sizes or pattern of bands | 2. Integrated or rearranged |
| CHEF gel | Restriction digest of genomic DNA | 1. Native sizes and pattern of bands | 1. Autonomous or integrated via |
| Southern blot | | | CEN fragment |
| | | 2. Altered sizes or pattern of bands | 2. Integrated or rearranged |
| | Native genomic DNA (no digest) | 1. MC band migrating ahead of | 1. Autonomous circles or linears |
| | | genomic DNA | present |
| | | 2. MC band co-migrating with | 2. Integrated |
| | | genomic DNA | |
| | | 3. >1 MC bands observed | 3. Various possibilities |
| Exonuclease | Exonuclease digestion of genomic DNA with | 1. Signal strength close to that w/o | 1. Autonomous circles present |
| | detection of circular MC by PCR, dot blot, or | exonuclease | |
| | restriction digest (optional), electrophoresis and | 2. No sgnal or signal strength lower | 2. Integrated |
| | southern blot (useful for circular MCs) | than w/o exonucldease | |
| MC rescue | Transformation of plant genomic DNA into E. coli | 1. Colonies isolated only from MC | 1. Autonomous circles present, |
| | followed by selection for antibiotic resistance genes | plants wit MC, not from controls; | native MC structure |
| | on MC | MC structure matches that of the | |
| | | paretal MC | |
| | | 2. Colonies isolated only fo MC | 2. Atuonomouse circles present, |
| | | plants with MCs, not from controls; | rearranged MC structure OR MCs |
| | | MC strctureerent from parental MC | integrated via centromere |
| | | | fragment. |
| | | 3. Colonies in MC modified plants | 3. Various possibilities |
| | | and and in controls | |
| PCR | PCR amplification of various parts of MC | 1. All MC parts detected | 1. Complete MC sequences |
| | | | present |
| | | 2. Subset of MC parts detected | 2. Partial MC sequences present |
| FISH | Detection of MC sequences in mitotic or meiotic | 1. MC seqeuences detected, free of | 1. Autonomous |
| | nuclei by fluorescence in situ hybridization | genome | |
| | | 2. MC sequences detected, | 2. Integrated |
| | | associated with genome | |
| | | 3. MC sequences detected, free and | 3. Both autonomous and |
| | | associated with genome | integrated MC sequences present |
| | | 4. No MC sequences detected | 4. MC DNA not visible by FISH |

Furthermore, MC structure can be examined by characterizing MCs rescued from min-chromosome-containing cells. Circular MCs that contain bacterial sequences for their selection and propagation in bacteria can be rescued from a mini-chromosome-containing plant or plant cell and re-introduced into bacteria. If no loss of sequences has occurred during replication of the MC in plant cells, the MC is able to replicate in bacteria and confer antibiotic resistance. Total genomic DNA is isolated from the min-chromosome-containing plant cells. The purified genomic DNA is introduced into bacteria (e.g., E. coli), and the transformed bacteria are plated on solid medium containing antibiotics to select bacterial clones modified with MC DNA. Modified bacterial clones are grown, the plasmid DNA purified (by alkaline lysis for example), and DNA analyzed, such as by restriction enzyme digestion and gel elcctrophoresis or by sequencing. Because plant-methylated DNA containing methylcytosine residues is degraded by wild-type strains of E. coli, bacterial strains (e.g., DH10B) deficient in the genes encoding methylation restriction nucleases (e.g., the mcr and mrr gene loci in E. coli) are best suited for this type of analysis. MC rescue can be performed on any plant tissue or clone of plant cells modified with a MC.

### I. Analyses of Transformed Plants

MC Autonomy Demonstration by In Situ Hybridization

While not necessary for the embodiments of the invention, it can be desirable to have a delivered MC maintained autonomously in the plant cell. To assess whether the MC is autonomous from the native plant chromosomes, or has integrated into the plant genome, in situ hybridizations can be used, such as FISH. In this assay, mitotic or meiotic tissue, such as root tips or meiocytes from the anther, possibly treated with metaphase arrest agents such as colchicines is obtained, and standard FISH methods are used to label both the centromere and sequences specific to the MC. For example, a Gossypium centromere is labeled using a probe from a sequence that labels all Gossypium centromeres, attached to one fluorescent tag, such as one that emits the red visible spectrum (ALEXA FLUOR® 568, for example (Invitrogen; Carlsbad, Calif.)), and sequences specific to the MC are labeled with another fluorescent tag, such as one emitting in the green visible spectrum (ALEXA FLUOR® 488, for example). All centromere sequences are detected with the first tag; only MCs are detected with both the first and second tag. Chromosomes are stained with a DNA-specific dye including but not limited to DAP1, Hocchst 33258, OliGreen, Giemsa YOYO, and TOTO. An autonomous MC is visualized as a body that shows hybridization signal with both centromere probes and MC specific probes and is separate from the native chromosomes.

Determination of Gene Expression Levels

The expression level of any gene present on the MC can be determined by several methods, such as for RNA, Northern Blot hybridization, Reverse Transcriptase-PCR, binding levels of a specific RNA-binding protein, in situ hybridization, or dot blot hybridization; or for proteins, Western blot hybridization, Enzyme-Linked Immunosorbant Assay (ELISA), fluorescent quantitation of a fluorescent gene product, enzymatic quantitation of an enzymatic gene product, immunohistochemical quantitation, or spectroscopic quantitation of a gene product that absorbs a specific wavelength of light.

Use of Exonuclease to Isolate Circular MC DNA from Genomic DNA

Exonucleases can be used to obtain pure MC DNA, suitable for isolation of MCs from E. coli or from plant cells. The method assumes a circular structure of the MC. A DNA preparation containing MC DNA and genomic DNA from the source organism is treated with exonuclease, for example lambda exonuclease combined with E. coli exonuclease I, or the ATP-dependent exonuclease (Qiagen, Inc.; Germantown, Md.). Because the exonuclease is only active on DNA ends, it specifically degrades the linear genomic DNA fragments, but does not degrade circular MC DNA. The result is MC DNA in pure form. The resultant MC DNA can be detected by a number of methods for DNA detection, such as PCR, dot blot, and Southern blot. Exonuclease treatment followed by detection of resultant circular MC can be used to determine MC autonomy.

Structural Analysis of MCs by BAC-End Sequencing

BAC-end sequencing procedures can be used to characterize MC clones for a variety of purposes, such as structural characterization, determination of sequence content, and determination of the precise sequence at a unique site on the chromosome (for example the specific sequence signature found at the junction between a centromere fragment and the vector sequences). In particular, this method is useful to prove the relationship between a parental MC and the MCs descended from it and isolated from plant cells by MC rescue, described above.

Methods for Scoring Meiotic MC Inheritance

A variety of methods can be used to assess the efficiency of meiotic MC transmission. In one embodiment of the method, gene expression of genes on the MC (marker genes or non-marker genes) can be scored by any method for detection of gene expression known to those skilled in the art, including visible scoring methods (e.g., fluorescence of fluorescent protein markers, scoring of visible phenotypes of the plant), scoring resistance of the plant or plant tissues to antibiotics, herbicides or other selective agents, measuring enzyme activity of proteins encoded by genes on the MC, measuring non-visible plant phenotypes, or directly measuring the RNA and protein products of gene expression using, for example, microarrays, northern blots, in situ hybridizations, dot blots, RT-PCR, western blots, immunoprecipitations, ELISAs, immunofluorescence and radio-immunoassays (RIAs). Gene expression can be scored in the post-meiotic stages of microspore, pollen, pollen tube or female gametophyte, or the post-zygotic stages such as embryo, seed, or progeny seedlings and plants. In another embodiment, the MC can de directly detected or visualized in post-meiotic, zygotic, embryonal or other cells in by detecting DNA (e.g., by FISH) or by MC rescue described above.

FISH Analysis of MC Copy Number in Meiocytes, Roots or Other Tissues of Min-Chromosome-Containing Plants

The copy number of the MC can be assessed in any cell or plant tissue by in situ hybridization, such as FISH. For example, FISH methods are used to label the centromere, using a probe that labels all chromosomes with one fluorescent tag, and to label sequences specific to the MC with another fluorescent tag. All centromere sequences are detected with the first tag; only MCs are detected with both the first and second tag. Nuclei are counter-stained with a DNA-specific dye, such as DAPI, Hoechst 33258, OliGreen, Giemsa YOYO, and TOTO. MC copy number is determined by counting the number of fluorescent foci that label with both tags.

Induction of Callus and Roots from Ad Chromosomal Plants Tissues for Inheritance Assays

MC inheritance is assessed using callus and roots induced from transformed plants. To induce roots and callus, tissues such as leaf pieces are prepared from min-chromosome-containing plants and cultured on a MS medium containing a cytokinin, e.g., 6-benzylaminopurinc (BA), and an auxin, e.g., α-naphthaleneacctic acid (NAA). Any tissue of A mini-chromosome-containing plant can be used for callus and root induction, and the medium recipe for tissue culture can be optimized using procedures known in the art.

Clonal Propagation of Min-Chromosome-Containing Plants

To produce multiple clones of plants from a MC-transformed plant, any tissue of the plant can be tissue-cultured for shoot organogenesis using regeneration procedures already described. Alternatively, multiple auxiliary buds can be induced from a MC-modified plant by excising the shoot tip, rooting the tip, and subsequently growing the tip into plant; each auxiliary bud can be rooted and produce a whole plant.

Scoring of Antibiotic- or Herbicide-Resistance in Seedlings and Plants (Progeny of Self- and Out-Crossed Transformants

Progeny seeds harvested from MC-modified plants can be scored for antibiotic- or herbicide resistance by seed germination under sterile conditions on a growth media (for example, MS medium) containing an appropriate selective agent for a particular selectable marker gene. Only seeds containing the MC can germinate on the medium and further grow and develop into whole plants. Alternatively, seeds can be germinated in soil, and the germinating seedlings can then be sprayed with a selective agent appropriate for a selectable marker gene. Seedlings that do not contain MC do not survive; only seedlings containing MC can survive and develop into mature plants.

Genetic Methods for Analyzing MC Performance

In addition to direct transformation of a plant with a MC, plants containing a MC can be prepared by crossing a first plant containing the functional, stable, autonomous MC with a second plant lacking the MC.

For example, pollen from A mini-chromosome-containing plant can be used to fertilize the stigma of a non-min-chromosome-containing plant. MC presence is scored in the progeny of this cross using the methods outlined above. In the second embodiment, the reciprocal cross is performed by using pollen from a non-min-chromosome-containing plant to fertilize the flowers of A mini-chromosome-containing plant. The rate of MC inheritance in both crosses can be used to establish the frequencies of meiotic inheritance in male and female meiosis. In the third embodiment, the progeny of one of the crosses just described are back-crossed to the non-min-chromosome-containing parental line, and the progeny of this second cross are scored for the presence of genetic markers in the plant's natural chromosomes as well as the MC. Scoring of a sufficient marker set against a sufficiently large set of progeny allows the determination oflinkage or co-segregation of the MC (or lack thereof) to specific chromosomes or chromosomal loci in the plant's genome. Genetic crosses performed for testing genetic linkage can be done with a variety of combinations of parental lines as are known to those skilled in the art.

### Field Evaluation of Transgenic Plants

Transgenic plant cell lines are regenerated, proliferated (to make genetically-identical replicates of each transgenic line), rooted, acclimated and used in field trials. For seed-bearing plants, seed is collected and segregated.

Descriptor data from typical plants of each transgenic accession plus tissue-cultured and regenerated from wild type and empty vector lines is collected at regular intervals over at least a year or more, depending on the type of plant transformed and is easily determined by one of skill in the art. Descriptors for which data can be collected include:
a. Morphological: flower color and size, seed size and weight, leaf color, leaf size, leaf margin teeth, number of branches from the main stem.
b. Growth: plant height and width, fresh and dry weight.
c. Chemical: farnesene, total resin, and total hydrocarbon content.
d. Phenology: first flower date, 50% bloom date, and seed maturity date (first seed harvest).
e. Seed production: total seed mass and weight
f. Imaging: digital images of entire plants, and of the leaves, flowers and seeds.
   Descriptor data (morphological, chemical, phonological, growth, production, and imaging) are collected, descriptive statistics performed and results analyzed. Seeds from selected transgenic lines that approach or meet the predetermined target are further propagated for large scale field trials. In this experiment, secondary input targets such as water requirements fertilizer requirement, and management practices are typically evaluated.

In the cases of increased terpenoid production, such as farnesene, NIR can be used to follow farnesene accumulation during the growing season. Plants from the field trials can also provide the materials needed for the initial extraction scale-up. Experiments can also be conducted to determine the stability of farnesene post-harvest in whole, chopped and chipped plants, and under a range of storage conditions varying time, temperature and humidity (Coffelt et al., 2009a; Cornish et al., 2000a; Cornish et al., 2000b; McMahan et al., 2006).

Processing of Transgenic Plants for Terpenoid Biofuel (Exemplified with Farnasene)

A. Extraction of Farnesene from Transgenic Feedstock

In previous studies, farnesene has been extracted from plant tissues using solid-phase microextraction (SPME)(Demyttenaere et al., 2004; Zini et al., 2003), subcritical CO₂ extraction (Rout et al., 2008), microwave-assisted solvent extraction (Serrano and Gallego, 2006), and two-stage solvent extraction (Pechous et al., 2005). Ionic liquid methods to extract aromatic and aliphatic hydrocarbons (Arce et al., 2008; Arce et al., 2007) can also be used for farnesene extraction. These techniques are useful on a small scale and will be evaluated for their efficacy in large scale operations. While chipped and ground dry plants, sometimes coupled with pellitization, have been effectively extracted using solvents, further disruption or poration of plant cell walls may increase extraction efficiency. The effect of various low cost pretreatment methods can be tested, including mild alkali or acid treatment, ammonia explosion, and steam explosion on extraction efficiency and product purity. Ultrasound-assisted extraction (Hernanz et al., 2008), liquid-liquid extraction at high pressure, and/or high temperature also may assist in solvent penetration (into the cell wall) and improve farnesene extraction.

Extraction methods can be tested and scaled through three stages: (1) individual plant analyses (OSU), (2) 0.5-5 L batch extractions, and (3) pilot scale extraction (CIW). Hexane, pentane and chloromethane (Edris et al., 2008; Mookdasanit et al., 2003), have been used as solvents for farnesene extraction, and acetone for resin extraction can also be tested. Alternative solvents, such as ethyl lactate and 2,3 butanediol, which allows large-scale operation at higher temperatures for effective solvent distribution ratio and selectivity. Samples of transgenic plants are dried and ground using lab or hammer mills, depending on the scale required. Following solvent selection, the 0.5-5 L experiments can initially use published biomass to solvent ratios and other parameters (Arce et al., 2007; Lai et al., 2005; Mookdasanit et al., 2003; Pechous et al., 2005; Serrano and Gallego, 2006; Zheng et al., 2004), including those previously researched at KSU (Ananda and Vadlani, 2010a; Ananda and Vadlani, 2010b), (Oberoi et al., 2010). The best temperature, agitation rate, extraction time, substrate:solvent ratio, moisture content of biomass, and temperature range obtained will be used to develop the design of experiments using response surface methodology (RSM)(Brijwani et al., 2010). The optimal parameters inform selection of the solvent system(s) in which farnesene exhibits the greatest solubility and the highest partition coefficient. The quality of the extractant can be analyzed with GC-MS, and farnesene content will be quantified using ¹H and ¹³C NMR (Zheng et al., 2004). These pilot studies will provide the relevant data for optimization of β-farnesene extraction in terms of solvent choice, solubility, yield, and solvent recoverability.

B. Conversion of Farnesene to Farnesane

The β-farnesene rich material from the extraction process can be hydrogenated via metal catalysis in a high-pressure Parr reactor. Since hydrogenation is an established process for conversion of olefins in chemical industry, various industrial-grade metal catalysts can be used (Gounder and Iglesia, 2011; Knapik et al., 2008; Zhang et al., 2003), such as palladium on carbon, and platinum, copper or nickel supported on alumina (or other acidic support). Catalyst loading (10-90 g/L), farnesene concentration (100-600 g/L), compressed hydrogen flow (40-100 psig), temperature (40-80° C.), and reaction time, will be optimized for efficient farnesane production. Catalytic efficiency can be characterized before and after hydrogenation using Fourier transform infrared spectroscopy (FTIR) and X-ray diffraction, with respect to carbon selectivity, operating parameters (temperature, pressure), reaction time, and final farnesane purity. Reaction completion can be determined using gas chromatography-flame ionization detection (GC-FID). These data will inform performance of medium scale (50-1000 L) trails for efficient farnesane production from transgenic plants.

### DEFINITIONS

“Min-chromosome-containing” plant or plant part means a plant or plant part that contains functional, stable and autonomous MCs. Min-chromosome-containing plants or plant parts can be chimeric or not chimeric (chimeric meaning that MCs are only in certain portions of the plant, and are not uniformly distributed throughout the plant). A mini-chromosome-containing plant cell contains at least one functional, stable and autonomous MC.

“Autonomous” means that when delivered to plant cells, at least some MCs are transmitted through mitotic division to daughter cells and are episomal in the daughter plant cells, i.e., are not chromosomally integrated in the daughter plant cells. Daughter plant cells that contain autonomous MCs can be selected for further propagation using, for example, selectable or screenable markers. During the introduction into a cell of a MC, or during subsequent stages of the cell cycle, there may be chromosomal integration of some portion or all of the DNA derived from a MC in some cells. The MC is still characterized as autonomous despite the occurrence of such events if a plant, plant part or plant tissue can be regenerated that contains episomal descendants of the MC distributed throughout its parts, or if gametes or progeny can be derived from the plant that contain episomal descendants of the MC distributed through its parts.

“Centromere” is any DNA sequence that confers an ability to segregate to daughter cells through cell division. This sequence can produce a transmission efficiency to daughter cells ranging from about 1% to about 100%, including to about 5%, 10%, 20%, 30%, 40%, 50%, 60%, 70%, 80%, 90% or about 95% of daughter cells. Variations in transmission efficiency can find important applications within the scope of the invention; for example, MCs carrying centromeres that confer 100% stability could be maintained in all daughter cells without selection, while those that confer 1% stability could be temporarily introduced into a transgenic organism, but later eliminated when desired. In particular embodiments of the invention, the centromere can confer stable transmission to daughter cells of a nucleic acid sequence, including a recombinant construct comprising the centromere, through mitotic or meiotic divisions, including through both mitotic and meiotic divisions. A plant centromere is not necessarily derived from plants, but has the ability to promote DNA transmission to daughter plant cells.

“Circular permutations” refer to variants of a sequence that begin at base n within the sequence, proceed to the end of the sequence, resume with base number one of the sequence, and proceed to base n−1. For this analysis, n can be any number less than or equal to the length of the sequence. For example, circular permutations of the sequence ABCD are: ABCD, BCDA, CDAB, and DABC.

“Co-delivery” refers to the delivery of two nucleic acid segments to a cell. The segments can be delivered simultaneously or sequentially. The segments can be the same kind of vector (e.g. two MCs) or different (e.g. a combination of MC, T-DNA, viral vector, plasmid vector, etc.). Alternatively, the segments can be co-delivered on a single vector.

“Consensus” refers to a nucleic acid sequence derived by comparing two or more related sequences. A consensus sequence defines both the conserved and variable sites between the sequences being compared. Any one of the sequences used to derive the consensus or any permutation defined by the consensus can be useful in construction of MCs.

“Exogenous” when used in reference to a nucleic acid, for example, refers to any nucleic acid that has been introduced into a recipient cell, regardless of whether the same or similar nucleic acid is already present in such a cell. An “exogenous gene” can be a gene not normally found in the host genome in an identical context, or an extra copy of a host gene. The gene can be isolated from a different species than that of the host genome, or alternatively, isolated from the host genome but operably linked to one or more regulatory regions that differ from those found in the unaltered, native gene. The gene can also be synthesized in vitro.

“Functional” when referring to a MC, centromere, nucleic acid, or polypeptide, for example, retains a biological and/or an immunological activity of native or naturally-occurring chromosome, centromere, nucleic acid, or polypeptide, respectively. When used to describe an exogenouse nucleic acid carried on an MC, “functional” means that the exogenous nucleic acid can function in a detectable manner when the MC is within a cell, such as a plant cell; exemplary functions of the exogenous nucleic acid include transcription of the exogenous nucleic acid, expression of the exogenous nucleic acid, regulatory control of expression of other exogenous nucleic acids, recognition by a restriction enzyme or other endonuclease, ribozyme or recombinase; providing a substrate for DNA methylation, DNA glycolation or other DNA chemical modification; binding to proteins such as histones, helix-loop-helix proteins, zinc binding proteins, leucine zipper proteins, MADS box proteins, topoisomerases, helicases, transposases, TATA box binding proteins, viral protein, reverse transcriptases, or cohesins; providing an integration site for homologous recombination; providing an integration site for a transposon, T-DNA or retrovirus; providing a substrate for RNAi synthesis; priming of DNA replication; aptamer binding; or kinetochore binding. If multiple exogenous nucleic acids are present within the MC, the function of one or preferably more of the exogenous nucleic acids can be detected under suitable conditions permitting function.

“Linker” refers to a DNA molecule, generally up to 50 or 60 nucleotides long, although linkers can be much larger, such as 100 bp, 1 kb, 100 kb, 1 Gb, etc., and composed of two or more complementary oligonucleotides that have been synthesized chemically, or excised or amplified from existing plasmids or vectors. In a preferred embodiment, this fragment contains one, or preferably more than one, restriction enzyme site for a blunt cutting enzyme and/or a staggered cutting enzyme, such as BamHl. One end of the linker is designed to be ligatable to one end of a linear DNA molecule and the other end is designed to be ligatable to the other end of the linear molecule, or both ends can be designed to be iigatable lo both ends of the linear DNA molecule.

A “mini-chromosome” (“MC”) is a recombinant DNA construct including a centromere and capable of transmission to daughter cells. A MC can remain separate from the host genome (as episomes) or can integrate into host chromosomes. The stability of this construct through cell division could range between from about 1% to about 100%, including about 5%, 10%, 20%, 30%, 40%, 50%, 60%, 70%, 80%, 90% and about 95%. The MC construct can be a circular or linear molecule. It can include elements such as one or more telomeres, origin of replication sequences, stuffer sequences, buffer sequences, chromatin packaging sequences, linkers and genes. The number of such sequences included is only limited by the physical size limitations of the construct itself. It can contain DNA derived from a natural centromere, although it can be preferable to limit the amount of DNA to the minimal amount required to obtain a transmission efficiency in the range of 1-100%. The MC can also contain a synthetic centromere composed of tandem arrays of repeats of any sequence, either derived from a natural centromere, or of synthetic DNA. The MC can also contain DNA derived from multiple natural centromeres. The MC can be inherited through mitosis or meiosis, or through both meiosis and mitosis. The term MC specifically encompasses and includes the terms “plant artificial chromosome” or “PLAC,” or engineered chromosomes or microchromosomes and all teachings relevant to a PLAC or plant artificial chromosome specifically apply to constructs within the meaning of the term MC.

“Non-protein expressing sequence” or “non-protein coding sequence” is defined herein as a nucleic acid sequence that is not eventually translated into protein. The nucleic acid can or can not be transcribed into RNA. Exemplary sequences include ribozymes or antisense RNA.

“Operably linked” is defined herein as a configuration in that a control sequence, e.g., a promoter sequence, directs transcription or translation of another sequence, for example a coding sequence. For example, a promoter sequence could be appropriately placed at a position relative to a coding sequence such that the control sequence directs the production of a polypeptide encoded by the coding sequence.

The term “plant,” as used herein, refers to any type of plant. Exemplary types of plants are listed below, but other types of plants will be known to those of skill in the art and could be used with the invention. Modified plants of the invention include, for example, dicots, gymnosperm, monocots, mosses, ferns, horsetails, club mosses, liver worts, homworts, red algae, brown algae, gametophytes and sporophytes of pteridophytes, and green algae.

A common class of plants exploited in agriculture are vegetable crops, including artichokes, kohlrabi, arugula, leeks, asparagus, lettuce (e.g., head, leaf, romaine), bok choy, malanga, broccoli, melons (e.g., muskmelon, watermelon, crenshaw, honeydew, cantaloupe), brussels sprouts, cabbage, cardoni, carrots, napa, cauliflower, okra, onions, celery, parsley, chick peas, parsnips, chicory, Chinese cabbage, peppers, collards, potatoes, cucumber plants (marrows, cucumbers), pumpkins, cucurbits, radishes, dry bulb onions, rutabaga, eggplant, salsify, escarole, shallots, endive, garlic, spinach, green onions, squash, greens, beet (sugar beet or fodder beet), sweet potatoes, swiss chard, horseradish, tomatoes, kale, turnips, or spices.

Other types of plants frequently finding commercial use include fruit and vine crops such as apples, grapes, apricots, cherries, nectarines, peaches, pears, plums, prunes, quince, almonds, chestnuts, filberts, pecans, pistachios, walnuts, citrus, blueberries, boysenberries, cranberries, currants, loganberries, raspberries, strawberries, blackberries, grapes, avocados, bananas, kiwi, persimmons, pomegranate, pineapple, tropical fruits, pomes, melon, mango, papaya, or lychee.

Modified wood and fiber or pulp plants of particular interest include, but are not limited to maple, oak, cherry, mahogany, poplar, aspen, birch, beech, spruce, fir, kenaf, pine, walnut, cedar, redwood, chestnut, acacia, bombax, alder, eucalyptus, catalpa, mulberry, persimmon, ash, honeylocust, sweetgum, privet, sycamore, magnolia, sourwood, cottonwood, mesquite, buckthorn, locust, willow, elderberry, teak, linden, bubinga, basswood or elm.

Modified flowers and ornamental plants of particular interest, include roses, petunias, pansy, peony, olive, begonias, violets, phlox, nasturtiums, irises, lilies, orchids, vinca, philodendron, poinscttias, opuntia, cyclamen, magnolia, dogwood, azalea, redbud, boxwood, Viburnum, maple, elderberry, hosta, agave, asters, sunflower, pansies, hibiscus, morning glory, alstromeria, zinnia, geranium, Prosopis, artemesia, clematis, delphinium, dianthus, gallium, coreopsis, iberis, lamium, poppy, lavender, leucophyllum, scdum, salvia, verbascum, digitalis, penstemon, savory, pythrethrum, or oenolhera. Modified nut-bearing trees of particular interest include, but are not limited to pecans, walnuts, macadamia nuts, hazelnuts, almonds, or pistachios, cashews, pignolas or chestnuts.

Many of the most widely grown plants are field crop plants such as evening primrose, meadow foam, corn (field, sweet, popcorn), hops, jojoba, peanuts, rice, safflower, small grains (barley, oats, rye, wheat, etc.), sorghum, tobacco, kapok, leguminous plants (beans, lentils, peas, soybeans), oil plants (rape, mustard, poppy, olives, sunflowers, coconut, castor oil plants, cocoa beans, groundnuts, oil palms), fibre plants (cotton, flax, hemp, jute), lauraceae (cinnamon, camphor), or plants such as coffee, sugarcane, cocoa, tea, or natural rubber plants.

Still other examples of plants include bedding plants such as flowers, cactus, succulents or ornamental plants, as well as trees such as forest (broad-leaved trees or evergreens, such as conifers), fruit, ornamental, or nut-bearing trees, as well as shrubs or other nursery stock.

Modified crop plants of particular interest in the present invention include soybean (Glycine max), cotton, canola (also known as rape), wheat, sunflower, sorghum, alfalfa, barley, safflower, millet, rice, tobacco, fruit and vegetable crops or turfgrasses. Exemplary cereals include maize, wheat, barley, oats, rye, millet, sorghum, rice triticale, secale, einkorn, spelt, emmer, teff, milo, flax, gramma grass, Tripsacum sp., or teosinte. Oil-producing plants include plant species that produce and store triacylglycerol in specific organs, primarily in seeds. Such species include soybean (Glycine max), rapeseed or canola (including Brassica napus, Brassica rapa or Brassica campestris), Brassica juncea, Brassica carinata, sunflower (Helianthus annuus), cotton (including Gossypium hirsutum), com (Zea mays), cocoa (Theobroma cacao), safflower (Carthamus tinctorius), oil palm (Elaeis guineensis), coconut palm (Cocos nucifera), flax {Linum usitatissimum), castor (Ricinus communis) or peanut (Arachis hypogaea).

“Sorghum” Sorghum bicolor (primary cultivated species), Sorghum almum, Sorghum amplum, Sorghum angustum, Sorghum rundinaceum, Sorghum brachypodum, Sorghum bulbosum, Sorghum burmahicum, Sorghum controversum, Sorghum drummondii, Sorghum carinatum, Sorghum exstans, Sorghum grande, Sorghum halepense, Sorghum interjectum, Sorghum intrans, Sorghum laxiflorum, Sorghum leiocladum, Sorghum macrospermum, Sorghum matarankense, Sorghum miliaceum, Sorghum nigrum, Sorghum nitidum, Sorghum plumosum, Sorghum propinquum, Sorghum purpureosericeum, Sorghum stipoideum, Sorghum timorense, Sorghum trichocladum, Sorghum versicolor, Sorghum virgatum, and Sorghum vulgare (including but not limited to the variety Sorghum vulgare var. sudanens also known as sudangrass). Hybrids of these species are also of interest in the present invention as are hybrids with othe members of the Family Poaceae.

“Guayule” means the desert shrub, Parthenium argentatum, native to the southwestern United States and northern Mexico and which produces polymeric isoprene essentially identical to that made by Hevea rubber trees (e.g., Hevea brasiliensis) in Southeast Asia.

“Plant part” includes pollen, silk, endosperm, ovule, seed, embryo, pods, roots, cuttings, tubers, stems, stalks, fiber (lint), square, boll, fruit, berries, nuts, flowers, leaves, bark, wood, whole plant, plant cell, plant organ, epidermis, vascular tissue, protoplast, cell culture, crown, callus culture, petiole, petal, sepal, stamen, stigma, style, bud, meristem, cambium, cortex, pith, sheath, or any group of plant cells organized into a structural and functional unit. In one preferred embodiment, the exogenous nucleic acid is expressed in a specific location or tissue of a plant, for example, epidermis, vascular tissue, meristem, cambium, cortex, pith, leaf, sheath, flower, root or seed.

“Promoter” is a DNA sequence that allows the binding of RNA polymerase (including but not limited to RNA polymerase I, RNA polymerase II and RNA polymerase III from eukaryotes), and optionally other accessory or regulatory factors, and directs the polymerase to a downstream transcriptional start site of a nucleic acid sequence encoding a polypeptide to initiate transcription. RNA polymerase effectively catalyzes the assembly of messenger RNA complementary to the appropriate DNA strand of the coding region.

A “promoter operably linked to a heterologous gene” is a promoter that is operably linked to a gene or other nucleic acid sequence that is different from the gene to that the promoter is normally operably linked in its native state. Similarly, an “exogenous nucleic acid operably linked to a heterologous regulatory sequence” is a nucleic acid that is operably linked to a regulatory control sequence to that it is not normally linked in its native state.

“Hybrid promoter” means parts of two or more promoters that are fused together to generate a sequence that is a fusion of the two or more promoters, that is operably linked to a coding sequence and mediates the transcription of the coding sequence into mRNA.

“Tandem promoter” means two or more promoter sequences each of that is operably linked to a coding sequence and mediates the transcription of the coding sequence into mRNA.

“Constitutive active promoter” means a promoter that allows permanent and stable expression of the gene of interest.

“Inducible promoter” means a promoter induced by the presence or absence of a biotic or an abiotic factor.

“Polypeptide” does not refer to a specific length of the encoded product and, therefore, encompasses peptides, oligopeptides, and proteins. “Exogenous polypeptide” means a polypeptide that is not native to the plant cell, a native polypeptide in that modifications have been made to alter the native sequence, or a native polypeptide whose expression is quantitatively altered as a result of a manipulation of the plant cell by recombinant DNA techniques.

“Pseudogene” refers to a non-functional copy of a protein-coding gene; pseudogenes found in the genomes of eukaryotic organisms are often inactivated by mutations and are thus presumed to be non-essential to that organism; pseudogenes of reverse transcriptase and other open reading frames found in retroelements are abundant in the centromeric regions of Arabidopsis and other organisms and are often present in complex clusters of related sequences.

“Regulatory sequence” refers to any DNA sequence that influences the efficiency of transcription or translation of any gene. The term includes sequences comprising promoters, enhancers and terminators.

“Repeated nucleotide sequence” refers to any nucleic acid sequence of at least 25 bp present in a genome or a recombinant molecule, other than a telomere repeat, that occurs at least two or more times and that are preferably at least 80% identical either in head to tail or head to head orientation either with or without intervening sequence between repeat units.

“Retroelement” or “retrotransposon” refers to a genetic element related to retroviruses that disperse through an RNA stage; the abundant retroelements present in plant genomes contain long terminal repeats (LTR retrotransposons) and encode a polyprotein gene that is processed into several proteins including a reverse transcriptase. Specific retroelements (complete or partial sequences (e.g., “retroelement-like sequence” and “retrotransposon-like sequence”) can be found in and around plant centromeres and can be present as dispersed copies or complex repeat clusters. Individual copies of retroelements can be truncated or contain mutations; intact retrolements are rarely encountered.

“Satellite DNA” refers to short DNA sequences (typically <1000 bp) present in a genome as multiple repeats, mostly arranged in a tandemly repeated fashion, as opposed to a dispersed fashion. Repetitive arrays of specific satellite repeats are abundant in the centromeres of many higher eukaryotic organisms.

“Screenable marker” is a gene whose presence results in an identifiable phenotype. This phenotype can be observed under standard conditions, altered conditions such as elevated temperature, or in the presence of certain chemicals used to detect the phenotype. The use of a screenable marker allows for the use of lower, sub-killing antibiotic concentrations and the use of a visible marker gene to identify clusters of transformed cells, and then manipulation of these cells to homogeneity. Examples of screenable markers include genes that encode fluorescent proteins that are detectable by a visual microscope such as the fluorescent reporter genes DsRed, ZsGreen, ZsYellow, AmCyan, Green Fluorescent Protein (GFP). An additional preferred screenable marker gene is lac.

The invention also contemplates novel methods of screening for min-chromosome-containing plant cells that involve use of relatively low, sub-killing concentrations of a selection agent (e.g., sub-killing antibiotic concentrations), and also involve use of a screenable marker (e.g., a visible marker gene) to identify clusters of modified cells carrying the screenable marker, after that these screenable cells are manipulated to homogeneity. A “selectable marker” is a gene whose presence results in a clear phenotype, and most often a growth advantage for cells that contain the marker. This growth advantage can be present under standard conditions, altered conditions such as elevated temperature, specialized media compositions, or in the presence of certain chemicals such as herbicides or antibiotics. Examples of selectable markers include the thymidine kinase gene, the cellular adenine phosphoribosyltransferase gene and the dihydryofolate reductase gene, hygromycin phosphotransferase genes, bar, neomycin phosphotransferase genes and phosphomannose isomerase (PMI), among others. Especially useful selectable markers in the present invention include genes whose expression confer antibiotic or herbicide resistance to the host cell, or proteins allowing utilization of a carbon source not normally utilized by plant cells. Especially useful are proteins conferring cellular resistance to kanamycin, G 418, paramomycin, hygromycin, bialaphos, and glyphosate for example, or proteins allowing utilization of a carbon source, such as mannose, not normally utilized by plant cells.

“Percent identity” can be obtained by the comparison of sequences and determination of percent identity between two nucleotide sequences can be accomplished using a mathematical algorithm. For example, the percent identity between two amino acid sequences can be determined using the Needleman and Wunsch algorithm that has been incorporated into the GAP program in the GCG software package (Needleman and Wunsch, 1970), using either a Blossum 62 matrix or a PAM250 matrix. Parameters are set so as to maximize the percent identity.

“Hybridizes under low stringency, medium stringency, and high stringency conditions” describes conditions for hybridization and washing. Hybridization is a well-known technique (Ausubel, 1987). Low stringency hybridization conditions means, for example, hybridization in 6× sodium chloride/sodium citrate (SSC) at about 45° C., followed by two washes in 0.5×SSC, 0.1% SDS, at least at 50° C.; medium stringency hybridization conditions means, for example, hybridization in 6×SSC at about 45° C., followed by one or more washes in 0.2×SSC, 0.1%) SDS at 55° C.; and high stringency hybridization conditions means, for example, hybridization in 6×SSC at about 45° C., followed by one or more washes in 0.2×SSC, 0.1% SDS at 65° C. Another non limiting example of stringent hybridization conditions are hybridization in a high salt buffer comprising 6×SSC, 50 mM Tris HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. Another non limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Another non limiting example of low stringency hybridization conditions are hybridization in 35% formamide, 5×SSC, 50 mM Tris HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris HCl (pH 7.4), 5 mM EDTA, and 0.1% SDS at 50° C. Other conditions of low stringency that may be used are well known in the art (e.g., as employed for cross species hybridizations).

“Stable” means that a MC can be transmitted to daughter cells over at least 8 mitotic generations. Some embodiments of MCs can be transmitted as functional, autonomous units for less than 8 mitotic generations, e.g., 1, 2, 3, 4, 5, 6, or 7. Preferred MCs can be transmitted over at least 8, 9, 10, 11, 12, 13, 14, 15, 16, 17, 18, 18, 19, 20, 21, 22, 23, 24, 25, 26, 27, 28, 29 or 30 mitotic generations, for example, through the regeneration or differentiation of an entire plant, and preferably are transmitted through meiotic division to gametes. Other preferred MCs can be further maintained in the zygote derived from such a gamete or in an embryo or endosperm derived from one or more such gametes. A “functional and stable” MC is one in that functional MCs can be detected after transmission of the MCs over at least 8 mitotic generations, or after inheritance through a meiotic division. During mitotic division, as occurs occasionally with native chromosomes, there can be some non-transmission of MCs; the MC can still be characterized as stable despite the occurrence of such events if A mini-chromosome-containing plant that contains descendants of the MC distributed throughout its parts can be regenerated from cells, cuttings, propagules, or cell cultures containing the MC, or if A mini-chromosome-containing plant can be identified in progeny of the plant containing the MC.

“Structural gene” is a sequence that codes for a polypeptide or RNA and includes 5′ and 3′ ends. The structural gene can be from the host into which the structural gene is transformed or from another species. A structural gene usually includes one or more regulatory sequences that modulate the expression of the structural gene, such as a promoter, terminator or enhancer. Structural genes often confer some useful phenotype upon an organism comprising the structural gene, for example, herbicide resistance. A structural gene can encode an RNA sequence that is not translated into a protein, for example a tRNA or rRNA gene.

“Synthetic,” when used in the context of a polynucleotide or polypeptide, refers to a molecule that is made using standard synthetic techniques, e.g., using an automated DNA or peptide synthesizer. Synthetic sequence can be a native sequence, or a modified sequence.

“Telomere” or “telomere DNA” refers to a sequence capable of capping the ends of a chromosome, thereby preventing degradation of the chromosome end, ensuring replication and preventing fusion to other chromosome sequences. Telomeres can include naturally occurring telomere sequences or synthetic sequences. Telomeres from one species can confer telomere activity in another species. An exemplary telomere DNA is a heptanucleotide telomere repeat TTTAGGG (SEQ ID NO:98; and its complement) found in the majority of plants.

“Trait” refers either to the altered phenotype of interest or the nucleic acid that causes the altered phenotype of interest.

“Transformed,” “transgenic,” “modified,” and “recombinant” refer to a host organism such as a plant into which an exogenous or heterologous nucleic acid molecule has been introduced, and includes whole plants, meiocytes, seeds, zygotes, embryos, endosperm, or progeny of such plants that retain the exogenous or heterologous nucleic acid molecule but that have not themselves been subjected to the transformation process.

When the phrase “transmission efficiency” of a certain percent is used, transmission percent efficiency is calculated by measuring MC presence through one or more mitotic or meiotic generations. It is directly measured as the ratio (expressed as a percentage) of the daughter cells or plants demonstrating presence of the MC to parental cells or plants demonstrating presence of the MC. Presence of the MC in parental and daughter cells is demonstrated with assays that detect the presence of an exogenous nucleic acid carried on the MC. Exemplary assays can be the detection of a screenable marker (e.g., presence of a fluorescent protein or any gene whose expression results in an observable phenotype), a selectable marker, or PCR amplification of any exogenous nucleic acid carried on the MC.

TABLE OF SOME ABBREVIATIONS

| Abbreviation | Definition |
|---|---|
| ASE | accelerated solvent extraction |
| AVP1 | Arabidopsis vacuolar pyrophosphatase-1 |
| CCE | carbon capture enhancement |
| CDPME | 4-(CDP)-2-C-methyl-D-erythritol |
| CTP | chloroplast targeting |
| DMAPP | dimethylallyl pyrophosphate |
| DXS | 1-deoxy-D-xylulose-5-phosphate synthase |
| EIMS | Electron Impact Mass Spectrometry |
| FME | farnesene metabolic engineering |
| FPP | farnesyl pyrophosphate |
| FPP | farnesyl pyrophosphate |
| FPPS | farnesyl pyrophosphate synthase |
| FTIR | Fourier transform infrared spectroscopy |
| GC | Gas chromatography |
| GC-FID | gas chromatography-flame ionization detection |
| GC-EIMS | Gas Chromatography with Electron Impact |
| | Mass Spectrometry |
| GPP | geranyl diphosphate |
| GPPS | geranyl diphosphate synthase |
| HMG-CoA | hydroxymethylglutaryl-coenzyme A |
| HPLC | High-pressure liquid chromatography |
| IPP | isopentenyl pyrophosphate |
| LC/MS | liquid chromatography-mass pectrometry |
| MC | mini-chromosome |
| MEP | methylerthritol phosphate pathway |
| MVA | mevalonic acid pathway |
| NIR | near infrared |
| OVP1 | Orzya vacuolar pyrophosphatase-1 |
| PMI | phosphomannose isomerase |
| RSM | response surface methodology |
| SPME | solid-phase microextraction |

### Examples

The following examples are meant to only exemplify the invention, not to limit it in any way. One of skill in the art can envision many variations and methods to practice the invention.

### Example 1

### Identification of Resin-Specific Promoters in Guayule

In order to identify resin-specific sequences quickly, Roche/454 GS-FLX and Illumina GAIIx platforms can be used to sequence the approximately 1100 MB guayule genome and its transcriptome. Two runs on the Roche instrument provide longer sequences (up to 600 bp, ^{˜}1.5 coverage on the genome). One half of a flowcell on the Illumina GAII platform provides shorter reads (paired-end, 100-150 bp, for ^{˜}30 fold genome coverage). A preliminary assembly of the guayule genome is performed by combining the 454 and Illumina reads, using Velvet or SOAPdenovo software analysis packages (publicly available), after quality trimming and removal of highly repetitive sequences from the dataset. The other half of the Illumina flow-cell can be used to sequence the guayule transcriptome, and provide 48 GB of transcriptome sequence. Transcripts can be assembled using the Rnnotator automated pipeline (Martin et al., 2010). Assemblies can be evaluated by running non-redundant protein BlastX (Altschul et al., 1990), and assembled transcripts can be characterized and annotated using Blast2GO (Conesa et al., 2005) using non-redundant databases and local Blast homology searches. Sequences of transcripts of genes involved in terpenoid synthesis can be then used to identify promoters. Resin vessel-specific promoters can be validated by expressing GFP or β-galactosidase genes in vivo, and then used to drive β-farnesene synthesis in either the cytosol or chloroplast of resin vessel cells.

### Example 2

### Guayule Mini-Chromosome Development

Developing mini-chromosomes using Chromatin, Inc.'s proprietary technology has been well described, for example, in U.S. Pat. Nos. 7,456,013, 7,227,057, 7,235,716, 7,226,782, 7,989,202, and 7,193,128.

To identify guayule centromeres, guayule genomic DNA from line AZ-2 is isolated from etiolated seedlings. A bacterial artificial chromosome (BAC) library is prepared in a modified pBeloBAC11 vector. The library is arrayed on nylon filters and hybridized with centromere-specific satellite or centromere-associated retrotransposon sequence probes. To identify probe sequences, guayule genomic DNA from line AZ-2 is subjected to a single sequencing run on Illumina (San Diego, Calif.; USA) GAIT analyzer or Roche (Pleasanton, Calif.; USA) GS-Titanium sequencer. Centromere probes are amplified from genomic DNA, cloned and characterized, and fluorescent in situ hybridization (FISH) analysis, such as described in (Carlson et al., 2007), is used to confirm centromere localization. About 50 BAC clones obtained from library screening is characterized at the molecular level and hybridized to guayule root tip metaphase chromosome spreads. The three BAC clones with highest content of centromere satellite repeats and retrotransposon sequences, and strongest and specific hybridization to centromere regions of metaphase chromosomes, are selected to build mini-chromosomes. Two forms of guayule are transformed: the apomyctic hybrid line AZ-101 and a rapidly growing, facultative, apomictic epitype selected from AZ-2.

### Example 3

### Construction of Farnesene Metabolic Engineering (FME) Gene Stacks in MCs

Gene-stacks encoding the β-farnesene synthesis pathway enzymes (such as those shown in Table 1) (the FME gene stack) are delivered on MCs, for example, by following the methods for mini-chromosome transformation in maize (Carlson et al., 2007) or by using traditional recombinant constructs, or a combination thereof. In addition, carbon capture enhancement constructs or individual β-farnesene gene control constructs are introduced into plant cells using modifications of Agrobacterium methods (Gao et al., 2005; Gurel et al., 2009; Zhao, 2006). In both microparticle and Agrobacterium delivery approaches, the phosphomannose isomerase (PMI) selectable marker (Reed et al., 2001) or any other suitable selectable marker, can be used to monitor transformation efficiency.

MCs used in transformation with the FME gene-stack can be constructed by Cre-Lox recombination of the FME gene stack from a donor plasmid into the Cre-Lox site contained within the modified pBeloBAC11 vector. Prior to transformation, the FME gene-stack containing MCs is digested with endonucleases at unique sites flanking the pBeloBAC11 vector backbone; followed by gel purification and ligation of the large gene-stack containing MC fragment. This allows transformation with, and production of transgenic lines containing, a backbone free version of the MC.

FME Gene Stack Constructs and MCs

In the first-generation sorghum constructs we used three approaches (constitutive promoter, tissue-specific promote, and subcellular protein targeting) to over-express the MVA and/or MEP pathway rate-limiting genes/proteins. Constitutive promoters could provide high gene expression in all tissues, which could result in an overall increase in farnesene production. However, constitutive production of β-farnesene may lead to toxic effects in cells that could be deleterious to plant health. To mitigate potential issues of toxicity, tissue-specific promoters preferentially expressed in stems or in lignifying tissues were also used. Expression of MVA pathway genes in lignifying tissues may restrain farnesene production to lignified tissues and prevent toxicity by reducing movement of β-farnesene from lignified cells to non-lignified cells essential for plant growth and development. The MEP pathway predominantly functions in chloroplasts; hence we have used chloroplast signal peptides to target MEP rate-limiting enzymes to chloroplasts for enhanced carbon flux.

TABLE A
FME Constructs

| Construct | Construct Name | Promoter type | Gene of Interest** |
|---|---|---|---|
| Sb1 | CHROM6192 | constitutive | Sc-HMGR (SEQ ID NO: 28) |
| | | constitutive | Sc-FPPS (SEQ ID NO: 29) |
| | | constitutive | Aa-β-FS (SEQ ID NO: 12) |
| | | constitutive | Os-VP1 (SEQ ID NO: 27) |
| Sb2 | CHROM6208 | ShOMT1* | Sc-HMGR (SEQ ID NO: 28) |
| | | ShOMT1* | Sc-FPPS (SEQ ID NO: 29) |
| | | ShOMT1* | Aa-β-FS (SEQ ID NO: 12) |
| Sb3 | CHROM6241 | ShOMT1* | Sc-HMGR (SEQ ID NO: 28) |
| | CHROM6248 | ShOMT1* | Sc-FPPS (SEQ ID NO: 29) |
| | CHROM6249 | ShOMT1* | Aa-β-FS (SEQ ID NO: 12) |
| Sb4 | CHROM6250 | ZmPEPC# | Cp Leader::Os-DXS1 (SEQ ID NO: 18) |
| | CHROM6231 | ZmPEPC# | Cp Leader::FPPS synthase (SEQ ID NO: 21) |
| | | ZmPEPC# | Cp Leader::β-FS (SEQ ID NO: 25) |
| “Sb5” | CHROM6208 | ShOMT1* | Sc-HMGR (SEQ ID NO: 28) |
| | CHROM6187 | ShOMT1* | Sc-FPPS (SEQ ID NO: 29) |
| | | ShOMT1* | Aa-β-FS (SEQ ID NO: 12) |
| | | ShOMT1* | Os-VP1 (SEQ ID NO: 27) |

*lignifying cell promoter
**appropriate terminators are also incorporated into the constructs for each gene; the constructs include an appropriate selectable marker under constitutive promoter control.
#leaf/stem tissue promoter

We completed construction of 12 FME gene constructs, generated four stacked plasmid gene constructs with 4-5 gene cassettes each and generated 4 mini-chromosomes containing a stacked gene construct (codon optimized) as listed in Table A. The following are a brief description of the first-generation FME gene stack constructs. The Sb1 construct constitutively expresses MVA pathway rate-limiting genes [yeast HMG CoA reductase (Sc-HMGR), yeast farnesyl diphosphate synthase (Sc-FPPS) and Artemisia β-farnesene synthase (Aa-β-FS)], and a rice vacuolar pyrophosphatase (Os-VP1) intended to maintain cytosolic pH. Sb2 contains the same rate-limiting MVA pathway genes as Sb1, but under the control of a lignifying cell-specific promoter. Sb3 is a mini-chromosome (MC)-based version of Sb2 intended to produce stable MC events. Sb4 uses a promoter to drive leaf and stem tissue expression of MEP pathway rate-limiting genes, whose products are targeted to the chloroplast. Sb5 was originally designed as a version of Sb2 possessing the addition of Os-VP1. However, Os-VP1 induced instability of the stacked genes in this construct. Hence Sb2 was co-transformed along with a second plasmid containing the Os-VP1 gene to achieve the goal of engineering transgenic plants containing the rate-limiting MVA pathway genes and the Os-VP1 gene. Transgenic plants containing the Sb2 and Sb5 gene cassettes can be compared to assess the importance of Os-VP1 in balancing potential cytosolic pH changes arising as a result of high rates of terpene biosynthesis.

The constructs from Table A were bombarded using standard techniques into callus of guayule, sugarcane, and sorghum. The results for sorghum and sugarcane are reported in Tables B and C.

TABLE B
FME sorghum bombardment results

| Construct/ | | | Drug | Drug selection | All genes of | |
|---|---|---|---|---|---|---|
| Set # | CHROM# | Plates | selection+ | PCR+ Events | interest+ | Regenerated |
| Sb1 | 6192 | 62 | 51 | 20 | 3 | |
| Sb2 | 6208 | 45 | 29 | 6 | 3 | |
| Sb3.1 | 6241 | 33 | 6 | 1 | 0 | |
| Sb3.2 | 6248 | 11 | 1 | 1 | 0 | |
| Sb3.3 | 6249 | 17 | 13 | 3 | 0 | |
| Sb3.4 | 6250 | 0 | 0 | 0 | 0 | |
| Sb4 | 6231 | 56 | 41 | 9 | 1 | |
| Sb5 | 6187 | 12 | 8 | 5 | 5 | |
| Sb9 | 6117, 6208, 6187 | 34 | 28 | 15 | 0 | |
| Controls | 6117 | 56 | 38 | 21 | 21 | 5 |
| Totals | | 326 | 215 | 81 | 33 | 5 |

TABLE C
FME sugarcane bombardment results

| Construct/ | | | Drug | Drug selection | | Tranfer to |
|---|---|---|---|---|---|---|
| Set # | CHROM# | Plates | selection+ | PCR+ Events | Regenerated | Greenhouse |
| So1 | 6117, 6192 | 48 | 169 | 169 | 64 | 51 |
| So2 | 6117, 6231 | 18 | 141 | 141 | 83 | 52 |
| So7 | 6312 | 18 | 42 | 42 | 26 | |
| So8 | 6117, 6208 | 42 | 125 | 125 | 97 | 54 |
| So9 | 6117, 6208, 6187 | 36 | 76 | 76 | 51 | 7 |
| So Controls | 6117 | 14 | 60 | 20 | 4 | 6 |
| So totals | | 320 | 1077 | 1038 | 528 | 203 |

Multiplex PCR (MxPCR) was used to confirm successful transformation of genes of interest into sorghum. Tissue from potential events was harvested at callus stage and subjected to DNA extraction according to standard phenol/chloroform extraction methods. A multiplex PCR was run using standard PCR conditions (59° C. annealing temperature; 35 amplification cycles) and primers designed to amplify fragments of several target genes and also contained primers for amplifying selectable markers as well as to an endogenous plant gene alpha dehydrogenase-1 (ADH1) as a positive control. For all PCRs the following control samples were included: wildtype sorghum (WT), the same wildtype sample spiked with purified plasmid that was used for the particle bombardment experiments (WT spiked), and water. All MxPCR samples were run on a 1.5% TAE gel alongside the 2-log ladder (2-L). The results are summarized in Table B.

### Example 4

### Identification of Gene-Stack Containing, Transformed Plant Cells

Transgenic events are characterized at the callus, and T0 plantlet/plant stage. The presence, structure, and copy number of the MC or gene construct in transformed callus and plant tissues is determined by multiplex or quantitative RT-PCR with primers specific to the genes in the gene stack; and/or hybridization of genomic DNA from transgenic tissue using specifically designed gene-specific probes on the QuantiGene Plex system (Affymetrix; Santa Clara, Calif., USA). Selected transgenic events with low copy number and intact gene stacks are analyzed by conventional genomic Southern blot hybridization with different MC-specific probes. For MC-transformed events, autonomous and/or integrated MCs can be identified by FISH to nuclei of transgenic callus or root tip cells from T0 plants with MC specific fluorescently labeled probes. In sorghum, PCR or hybridization based assays is used to characterize T1/T2 progeny from crosses.

Reverse Transcriptase PCR (RT-PCR) was used to confirm expression of target transgenes in transformation events that were previously identified according to MxPCR methods described in Example 4. Leaf tissue of transgenic and control plants was harvested at various developmental stages and maintained at −80° C. RNA was extracted from the leaf tissue using the Qiagen (Valencia, Calif.; USA) RNeasy Plant Mini kit according to the manufacturer's instructions, including a DNAse treatment step. Reverse transcription was performed using Life Technologies (Grand Island, N.Y.; USA) SuperScript® III First Strand Synthesis kit according to the manufacturer's instructions. PCR was conducted using standard PCR conditions (59° C. annealing temperature; 35 amplification cycles) and primers were designed to amplify fragments the genes of interest. For all PCRs the following control samples were included: wildtype sugarcane and a positive control spike sample that consisted of purified plasmid that was used for the particle bombardment experiments. The spiked positive control was not DNAse treated. Two PCRs per sample were conducted: first without the addition of reverse transcriptase and second including the addition of reverse transcriptase. For the Sol experiments (see Table C), five plants were found to express some or all of the genes of interest; for Sot experiments (see Table C), five plants were also found to express some or all of the genes of interest. Finally, for Sob experiments, three plants were also found to express some or all of the genes of interest.

### Example 5

### Analyses of Transformed Plant Cells and Plants

The expression level and functionality of the delivered FME or carbon metabolic engineering genes, whether delivered on MCs or using Agrobacterium constructs, is determined using QRT-PCR, immunoblotting, and enzymatic activity assays; confirmed by LC-MS and terpenoid fingerprinting. Since tissue-specific promoters can be used for trait gene expression, all expression analysis can be performed on T0, T1, or T2 plants of the appropriate developmental stage and in the correct tissue, such as root, stem, leaf, seed, or progeny seedlings. In sorghum we will characterize genetic stability and transmission by crossing fertile transgenic plants or by reciprocal crosses with non-transgenic lines. An example of an assay that measures sesquiterpene and farnesene production is shown in Example 7.

After transgenic lines with MC gene stacks are generated, their ability to produce increased amounts of β-farnesene is quantified using metabolite analysis, comparing vector controls with accessions produced from at least 10 independent transformation events per transgenic strategy. Guayule and sorghum transgenic plants are grown and then rooted and grown in greenhouses. Replicates are harvested at monthly intervals and analyzed for β-farnesene, and resin content, using high-throughput accelerated solvent extraction (ASE) (Pearson et al., 2010; Salvucci et al., 2009), transitioning to near-infrared (NIR) analyses (Cornish et al., 2004). Additionally, the terpenoid “fingerprint” of resin composition from transgenic lines is determined by using mass spectrometry and high-pressure liquid chromatography (HPLC) to identify all terpenoid molecules present. Finally, gas chromatography (GC) and nuclear magnetic resonance (NMR) can be used to quantify the precise (mg/mL resin) quantities of specific terpene moieties. These data are used to calculate changes in pathway flux and the degree to which carbon has been routed into different substrate pools which, in turn, indicate the location of any additional rate-limiting steps to be targeted for additional genetic engineering.

Further analysis of transgenic plants can include the following, exemplified for guayule and sorghum: Transgenic, apomyctic guayule lines are regenerated, proliferated (to make genetically-identical replicates of each transgenic line), rooted and acclimated for governmental agency-approved field trials, such as done for three past transgenic guayule trials (Veatch et al., 2005). Sexually-competent guayule transgenics reach field trials the following spring. Plants are started in greenhouses in December-January in pots, and transplanted into the field in March/April. Seed is collected and segregated from all plants from the spring, summer and fall seed-set. Weed barriers are used to reduce labor and decrease competition between seedlings and weeds, and fields are irrigated as needed

Descriptor data from five typical plants of each transgenic accession plus tissue-cultured and regenerated from wild type and empty vector lines are collected every two months (starting at six months) for two years. Guayule descriptors for which data can be collected include:
a. Morphological: flower color and size, seed size and weight, leaf color, leaf size, leaf margin teeth, number of branches from the main stem.
b. Growth: plant height and width, fresh and dry weight every two months starting at six months for two years for two years.
c. Chemical: farnesene, total resin, and total hydrocarbon (resin+rubber) content can be quantified bimonthly, starting at six months, for two years.
d. Phenology: first flower date, 50% bloom date, and seed maturity date (first seed harvest) for two years.
e. Seed production: total seed mass and the weight/1000 from spring bloom after one and two years. Imaging: digital images can be made of entire plants every two months starting at six months for two years (the same tagged plants), and of the leaves, flowers and seeds.

Descriptor data (morphological, chemical, phonological, growth, production, and imaging) are collected, descriptive statistics performed and results (including images) entered into the public Germplasm Resources Information Network (GRIN). Seeds from selected transgenic lines that approach or meet the biofuel target are further propagated for large scale field trials. Secondary input targets, such as low irrigation requirements (≦22 inches/year) and low fertilizer requirement (N≦179 lbs/acre; P≦62 lbs/acre and K≦50 lbs/acre), and management practices are evaluated.

For transgenic sorghum, lines are initially grown in the greenhouse. Phenotypic data such as leaf color, days to flowering and disease/pest resistance or susceptibility can be recorded on individual primary transgenic plants. Plant height, fresh and dry weight of the plants is collected at maturity. β-farnesene and total terpenoid production is monitored as described above. Selected transgenic lines are also crossed to appropriate male sterile (A) lines, restorer (R) lines or maintainer (B) lines in order to utilize the cytoplasmic male sterility system used in commercial sorghum hybrid seed production. MC and gene-stack or construct performance and expression of encoded transgenes in different backgrounds is characterized with the methods outlined above. After initial screening, selected transgenic lines are backcrossed in the greenhouse to select sweet and forage sorghum lines to recover transgenic lines in different genotypes. Sorghum transgenic lines transformed with FME MCs can be crossed to transgenic lines transformed with Agrobacterium CCE vectors to evaluate increased feedstock production integration with β-farnesene enrichment provided by the FME MCs

Regulated field trials of the transgenic, sorghum T2 and T3 generation lines are conducted at an appropriate sorghum breeding facility. Each transgenic line is evaluated for its agronomic performance, total biomass yield and farnesene content under regulated conditions. Such protocols include proper isolation distances to avoid any transgenic plant material mixing with non-transgenic material. Seeds are planted in a weed-free bed after soil temperatures reach 65° F. or higher. Plants can be irrigated as needed with ≦22 inches of water during the growing season and the fertilizer input that does not exceed N:P:K levels of 179:62:50 lbs/acre. NIR is used to follow farnesene accumulation during the growing season. The trial is grown for a single cut at the end of the season. Harvesting occurs on late October early November depending on total biomass accumulation. Plants from the field trials also provide the materials needed for initial extraction scale-up experiments. Experiments to determine the stability of farnesene post-harvest in whole, chopped and chipped plants, and under a range of storage conditions varying time, temperature and humidity are performed (Coffelt et al., 2009a; Cornish et al., 2000a; Cornish et al., 2000b; McMahan et al., 2006).

### Example 6

### Extraction of Farnesene from Plant Materials

In previous studies, farnesene has been extracted from plant tissues using solid-phase microextraction (SPME) (Demyttenaere et al., 2004; Zini et al., 2003), subcritical CO₂ extraction (Rout et al., 2008), microwave-assisted solvent extraction (Serrano and Gallego, 2006), and two-stage solvent extraction (Pechous et al., 2005). Ionic liquid methods to extract aromatic and aliphatic hydrocarbons (Arce et al., 2008; Arce et al., 2007) can also be used for farnesene extraction. These techniques are useful on a small scale and can be evaluated for their efficacy in large scale operations. While chipped and ground dry plants, sometimes coupled with pellitization, have been effectively extracted using solvents, further disruption or poration of plant cell walls can increase extraction efficiency. The effect of various pre-treatment methods, including mild alkali or acid treatment, ammonia explosion, and steam explosion on extraction efficiency and product purity are tested. Ultrasound-assisted extraction (Hernanz et al., 2008), liquid-liquid extraction at high pressure, and/or high temperature also may assist in solvent penetration (into the cell wall) and improve farnesene extraction.

Extraction methods are tested and scaled through three stages: (1) individual plant analyses, (2) 0.5-5 L batch extractions, and (3) pilot scale extraction. Hexane, pentane and chloromethane (Edris et al., 2008; Mookdasanit et al., 2003) have been used as solvents for farnesene extraction, and acetone for resin extraction. Alternative solvents, such as ethyl lactate and 2,3 butanediol, are also tested, as they permit large-scale operation at higher temperatures for effective solvent distribution ratio and selectivity. Samples of sorghum and guayule are dried and ground using lab or hammer mills, depending on the required scale. Following solvent selection, the 0.5-5 L experiments initially use published biomass:solvent ratios and other published parameters (Arce et al., 2007; Lai et al., 2005; Mookdasanit et al., 2003; Pechous et al., 2005; Serrano and Gallego, 2006; Zheng et al., 2004), including (Ananda and Vadlani, 2010a; Ananda and Vadlani, 2010b), (Oberoi et al., 2010). The optimal temperature, agitation rate, extraction time, substrate:solvent ratio, moisture content of biomass, and temperature range obtained are used to develop experimental design using response surface methodology (RSM) (Brijwani et al., 2010). The optimal parameters will inform selection of the solvent system (s) in which farnesene exhibits the greatest solubility and the highest partition coefficient. The quality of the extractant is analyzed with GC-MS, and farnesene content is quantified using ¹H and ¹³C NMR (Zheng et al., 2004). These pilot studies provide the relevant data for optimization of β-farnesene extraction in terms of solvent choice, solubility, yield, and solvent recoverability. These data are used for process simulation and sensitivity studies, and they provide a vital framework for continuous extraction feasibility studies and semi-works runs.

### Example 7

### Quantitation of Sesquiterpene Levels

Overall, 113 transgenic sugarcane events were confirmed for presence of the target genes of interest (e.g., see Table C) and were selected for GC, GC-MS and LC-MS analyses, including using the assays described below, “Measuring sesquiterpenes in plant samples”. A summary of these analyses is shown in Table D. A subset of 31 of these samples was analyzed by LC-MS for the MVA and MEP pathway intermediates MVA, MVAP, MVAPP, CDPME, MEP, DXP, and IPP.

Measuring Sesquiterpenes in Plant Samples—Method

As an example of a quantitative assay for measuring sesquiterpenes, the following assay was developed. Plant samples are flash-frozen, triple ground to powder in liquid nitrogen, and extracted in dichloromethane (see also Example 6). Samples are then concentrated, separated using an HP-5 5% phenylmethylsiloxane column, and terpenes are both identified and quantified using mass spectral fingerprints. Additional protocol validation studies included (a) determination of the minimal content of sesquiterpenes detectable in plant extracts using 2 μg/mL concentration of the trichlorobenzene internal standard, (b) an extraction recovery determination of an externally spiked farensene sorghum stem sample, and (c) implementation of a method to concentrate plant extracts for assay. To define the lower limit of detection of farnesene in sorghum extracts using the above GC-EIMS methodology, a commercially obtained sample of farnesene isomers at 1.0 μg/mL was added to the extract (2 mL) of a sorghum stem sample. The resulting solution was serially diluted to provide additional 0.1 μg/mL, 0.05 μg/mL, and 0.01 μg/mL concentrations of farnesenes with a constant 2 μg/mL concentration of the trichlorobenzene internal standard. Each solution was subjected to GC-EIMS analysis under the optimized conditions described above for the guayule plant samples. Simple visualization of the total ion count traces indicated that the mixture containing farnesenes, with the major farnesene peak at 6.48 minutes retention time, was readily detectable at 0.05 μg/mL, but not so at 0.01 μg/mL, providing a limit of detection of sesquiterpenes at ca. 10⁻⁵% of dry plant material. Based on the terpenoid profiling studies conducted in sorghum and guayule it could be concluded that mono- or sesquiterpenes are not present above ca. 0.0001% by dry mass in non-transformed sorghum plant samples.

A commercially obtained sample of farnesene isomers (2.0 μg) was directly injected into a sorghum stem sample (ca. 1 g). The plant material was allowed to stand at room temperature for approximately 24 h before being chopped and extracted for 48 h with ethyl acetate (2 mL). The extract was filtered and analyzed as usual by GC-EIMS. The farnesenes were detected at about 64% of the injected amount (the crude condition of the commercial farnesene sample limits the quantification accuracy).

Measuring Sesquiterpenes in Plant Samples—Transgenic Sugarcane.

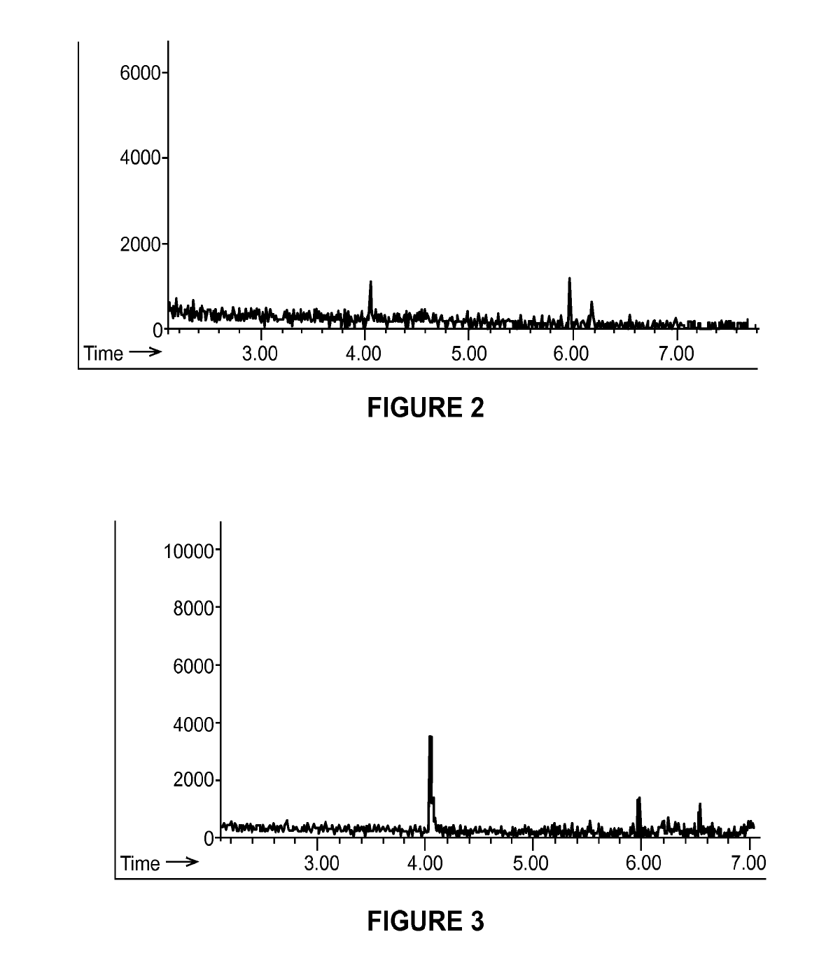

Using the method described immediately above, 113 events were analyzed for sesquiterpene production, of which 26 were identified as accumulating farnesenes or farnesene-like sesquiterpenes. Of these, 6 were unambiguously identified by mass spectrometry. Representative GC-MS total ion chromatograms from two positive events (AL2 and AL414) are shown in FIGS. 2 and 3. The remaining 20 sesquiterpene-containing samples tentatively identified by GC retention time are awaiting confirmation by GC-MS. In all cases, levels of sesquiterpenes did not appear to exceed 5 μg/gFW.

TABLE D
Summary of constructs and events analyzed
for production of farnesene

| Construct | | Plants | Farnesene |
|---|---|---|---|
| or Set # | CHROM# | Analyzed | Positive |
| So1 | 6117, 6192 | 29 | 8 |
| So2 | 6117, 6231 | 18 | 7 |
| So8 | 6117, 6208 | 22 | 4 |
| So9 | 6117, 6208, 6187 | 2 | |

Quantification of MVA and MEP Pathway Intermediates in Transgenic Sugarcane

In conjunction with end-point analyses to determine the effect of metabolic engineering on overall sesquiterpene production, we also completed MVA and MEP pathway analyses of our sugarcane transgenic lines. These analyses will allow us to determine whether overexpression of FME enzymes results in increased production of their corresponding metabolite, while at the same time allowing us to identify and rectify any metabolic “bottlenecks” (indicated by a build-up of a pathway intermediate) our engineering has created.

As our initial metabolic engineering approaches have focused on manipulations of the MVA pathway, we first quantified the intermediates of this pathway. Analysis of MVA pathway intermediates in leaf tissues indicates that transformation of sugarcane with the FME rate-limiting genes HMGR, FPPS, and bFS in conjunction with the H+-pyrophosphatase OsVP1, results in increased levels of MVA pathway metabolites, as seen in samples AL2, AL14, AL15, and AL22 below (Table E). Table E shows the levels of sesquiterpenes, MVA metabolites, and MEP metabolites that were analyzed via GC-EIMS (for sesquiterpenes) or LC-MS/MS (MEP and MVA intermediates). Levels of metabolites are presented as ug/g plant tissue. AL128-B and AL128 S serve as controls for: AL2, AL14, AL15, and AL31; AL334 serves as the control for AL414, AL422, AL40, AL56, AL98, AL172, AL593, and AL597. Double lines are used to separate different genetic constructs. Samples with elevated levels of sesquiterpenes are shown in boldface.

In the AL2, AL14, AL15, and AL22 samples, increased FME gene expression resulted in increased levels of either MVAPP, or both MVAP and MVAPP. These data correlate well with our sesquiterpene end-point analyses, where samples over-expressing the same gene cassette showed the highest levels of sesquiterpene accumulation compared to control samples.

When we analyzed MVA pathway intermediates in our second group of transgenics (where the samples consisted of combined leaf and whorl tissues), the observed results again matched well with our GC-EIMS end-of-pathway analyses. Our GC-EIMS data indicated that sugarcane overexpressing chloroplast-targeted FME genes exhibited slightly increased levels of sesquiterpenes; and this trend was reflected in our MVA pathway intermediate analyses. Samples AL381, AL403, and AL414, which have been engineered to constitutively express the chloroplast-targeted FME enzymes DXS, bFS, and FPPS, exhibit higher levels of MVA, MVAPP, or both, compared to control samples. Interestingly, sample AL98, which expresses the rate-limiting FME genes HMGR, FPPS, and bFS in a lignin-specific fashion also exhibited slightly higher levels of MVAP compared to control.

While our initial metabolic engineering efforts focused on manipulations of the MVA pathway, it is possible that our efforts may also have either directly or indirectly altered carbon partitioning through the MEP pathway. To determine the effect of our manipulation of FME genes on MEP metabolite levels, we quantitated these in transgenic sugarcane tissues. As with the MVA metabolite data presented above, the MEP metabolite data correlated well with our end-of-pathway GC-EIMS analyses. As with both sesquiterpenes and MVA metabolites, we observed increased MEP metabolite accumulation in the leaves of plants expressing HMGR, FPPS, bFS, and Os-VP1. In almost all cases, this was observed as increases in DXP levels, although some lines (AL31), increased levels of MEP were also observed. Interestingly, we observed no increases in MEP levels in sugarcane plants transformed with chloroplastically targeted DXS. However, this may be due to endogenous post-translational feedback-regulatory mechanisms and/or endogenous metabolic pathways present in the chloroplast (where DXS orthologs would normally localize) exhibiting tighter control of the levels of DXP in its native environment.

Taken together, our GC-EIMS and LC-MS/MS quantitation of MEP metabolites, MVA metabolites, and end-of-pathway sesquiterpenes indicate that three genetic constructs can increase the production of sesquiterpenes or sesquiterpene metabolites. These constructs are: 1. HMGR, FPPS, bFS, and Os-VP1 expressed under a constitutive promoter; 2. HMGR, FPPS, and bFS expressed under a lignin-specific promoter; and 3. DXS, bFS, and FPPS targeted to the chloroplast under a constitutive promoter. Of these three groups in these reported experiments, only the HMGR-FPPS-bFS-OsVP1 and chloroplast localized DXS-bFS-FPPS cassettes resulted in increased accumulations of sesquiterpenes. These data suggest that elimination of potentially toxic metabolic by-products, either through hydrolysis/extrusion (OsVP1) or sequestration (chloroplast localization) is important allowing increased terpenoid accumulation. The HMGR-FPPS-bFS-OsVP1 cassette generated the greatest number of plants with increased sesquiterpene levels, as well as the greatest number of plants with increased levels of MVA metabolites. Additionally, in AL2 and AL15, increased levels of both MVA intermediates and sesquiterpenes were observed. More importantly, a third member of this group, AL14, demonstrated increases in MEP metabolite levels, MVA metabolite levels, and sesquiterpenes, making this construct (as well as AL2 and AL15) an ideal candidate for farnesene metabolic engineering in sorghum.

TABLE E
Summary of GC-eiMS and LC-MS/MS terpene metabolite analyses in transegenic sugarcane.

| | | | | MVA | MVAP | MVAPP | CDPME | MEP | DXP | |
|---|---|---|---|---|---|---|---|---|---|---|
| | | | Sesqui- | (ug/ | (ug/ | (ug/ | (ug/ | (ug/ | (ug/ | IPP |
| | Event | | Terpenes | gFW) ± | gFW) ± | gFW) ± | gFW) ± | gFW) ± | gFW) ± | (ug/gFW) ± |
| | Name | Construct; expression mode | (ug/gFW) | SD | SD | SD | SD | SD | SD | SD |
| Con- | AL128 | Wild-type Non-transformed | <0.2 | 4.0075 ± | BLD | BLD | BLD | BLD | BLD | 9.4542 ± |
| trols | B | | | 1.5255 | | | | | | 1.2601 |
| | AL128 | Wild-type Non-transformed | <0.2 | 5.1389 ± | BLD | BLD | BLD | BLD | BLD | 10.8985 ± |
| | S | | | 2.6223 | | | | | | 1.6861 |
| | AL344 | Vector control | <0.2 | 6.6487 ± | BLD | BLD | BLD | 3.2771 ± | BLD | 27.9829 ± |
| | | | | 0.4631 | | | | 0.1234 | | 1.6479 |
| | AL2 | Sc-HMGR, Sc-FPPS, Aa-bFS, Os- | 0.5 | 2.7472 ± | BLD | 1.1709 ± | BLD | BLD | BLD | 8.5734 ± |
| | | VP1; Constitutive | | 0.5355 | | 0.4389 | | | | 1.1140 |
| | AL14 | Sc-HMGR, Sc-FPPS, Aa-bFS, Os- | 0.5 | 2.2865 ± | BLD | 1.3454 ± | BLD | BLD | 0.4642 ± | 7.3020 ± |
| | | VP1; Constitutive | | 0.2286 | | 0.3619 | | | 0.0162 | 0.2968 |
| | AL15 | Sc-HMGR, Sc-FPPS, Aa-bFS, Os- | 0.5 | 2.6155 ± | 0.0884 ± | 1.1021 ± | BLD | BLD | BLD | 11.3692 ± |
| | | VP1; Constitutive | | 0.5707 | 0.0329 | 0.3196 | | | | 1.5128 |
| | AL31 | Sc-HMGR, Sc-FPPS, Aa-bFS, Os- | 0.5 | 4.6104 ± | BLD | BLD | BLD | 0.1150 ± | BLD | 9.0451 ± |
| | | VP1; Constitutive | | 2.3258 | | | | 0.0123 | | 0.1671 |
| | AL414 | CTP-Os-DXS, CTP-Aa-bFS, CTP- | Trace | 2.2139 ± | BLD | 0.5695 ± | BLD | 0.3626 ± | BLD | 6.0532 ± |
| | | Sc-FPPS; constitutive | | 0.1642 | | 0.0551 | | 0.0970 | | 0.2609 |
| | AL422 | CTP-Os-DXS, CTP-Aa-bFS, CTP- | Trace | 2.2494 ± | BLD | BLD | BLD | 0.3750 ± | BLD | 4.1305 ± |
| | | Sc-FPPS; constitutive | | 0.1584 | | | | 0.0727 | | 0.0431 |
| | AL40 | Sc-HMGR, Sc-FPPS, Aa-bFS; | <0.2 | 1.5527 ± | BLD | BLD | BLD | BLD | BLD | 11.2197 ± |
| | | lignifying cell specific | | 0.1450 | | | | | | 0.1665 |
| | AL56 | Sc-HMGR, Sc-FPPS, Aa-bFS; | <0.2 | 1.1836 ± | BLD | BLD | BLD | BLD | BLD | 7.7934 ± |
| | | lignifying cell specific | | 0.3738 | | | | | | 0.2796 |
| | AL98 | Sc-HMGR, Sc-FPPS, Aa-bFS; | <0.2 | 4.2745 ± | 0.970 ± | BLD | BLD | BLD | BLD | 13.2164 ± |
| | | lignifying cell specific | | 0.4311 | 0.0080 | | | | | 1.9582 |
| | AL172 | Sc-HMGR, Sc-FPPS, Aa-bFS; | <0.2 | 1.1788 ± | BLD | BLD | BLD | BLD | BLD | 8.4835 ± |
| | | lignifying cell specific | | 0.0912 | | | | | | 0.0392 |

BLD, below detection.

### Example 8

### Conversion of Farnesene to Farnesane

The β-farnesene-rich material from the extraction process is hydrogenated via metal catalysis in a high-pressure Parr reactor. Since hydrogenation is an established process for conversion of olefins in chemical industry, various industrial-grade metal catalysts can be and are used (Gounder and Iglesia, 2011; Knapik et al., 2008; Zhang et al., 2003), such as palladium on carbon, and platinum, copper or nickel supported on alumina (or other acidic support). Catalyst loading (10-90 g/L), farnesene concentration (100-600 g/L), compressed hydrogen flow (40-100 psig), temperature (40-80° C.), and reaction time, are optimized for efficient farnesane production. Catalytic efficiency can be characterized before and after hydrogenation using Fourier transform infrared spectroscopy (FTIR) and X-ray diffraction, with respect to carbon selectivity, operating parameters (temperature, pressure), reaction time, and final farnesane purity. Reaction completion is determined using gas chromatography-flame ionization detection (GC-FID). These data will inform performance of medium scale (50-1000 L) trails for efficient farnesane production from transgenic plants.

### LITERATURE CITATIONS

Altschul, S. F., W. Gish, W. Miller, E. W. Myers, and D. J. Lipman. 1990. Basic local alignment search tool. J Mol Biol. 215:403-410.
Ananda, N., and P. V. Vadlani. 2010a. Fiber Reduction and Lipid Enrichment in Carotenoid-Enriched Distillers Dried
Grain with Solubles Produced by Secondary Fermentation of Phaffia rhodozyma and Sporobolomyces roseus. Journal of Agricultural and Food Chemistry. 58:12744-12748.
Ananda, N., and P. V. Vadlani. 2010b. Production and optimization of carotenoid-enriched dried distiller's grains with solubles by Phaffia rhodozyma and Sporobolomyces roseus fermentation of whole stillage. Journal of industrial microbiology & biotechnology. 37:1183-1192.
Aoyama, T., and N. H. Chua. 1997. A glucocorticoid-mediated transcriptional induction system in transgenic plants. Plant J. 11:605-612.
Arce, A., M. J. Earle, H. Rodriguez, K. R. Seddon, and A. Soto. 2008. 1-Ethyl-3-methylimidazolium bis{(trifluoromethyl)sulfonyl}amide as solvent for the separation of aromatic and aliphatic hydrocarbons by liquid extraction-extension to C-7- and C-8-fractions. Green Chemistry. 10:1294-1300.
Arce, A., A. Pobudkowska, 0. Rodriguez, and A. Soto. 2007. Citrus essential oil terpenless by extraction using 1-ethyl-3-methylimidazolium ethylsulfate ionic liquid: Effect of the temperature. Chemical Engineering Journal. 133:213-218.
Ausubel, F. M. 1987. Current protocols in molecular biology. Greene Publishing Associates; J. Wiley, order fulfillment, Brooklyn, N. Y.
Media, Pa. 2 v. (loose-leaf) pp.
Bach, T. J., A. Boronat, C. Caelles, A. Ferrer, T. Weber, and A. Wettstein. 1991. Aspects Related to Mevalonate Biosynthesis in Plants. Lipids. 26:637-648.
Bell-Lelong, D. A., J. C. Cusumano, K. Meyer, and C. Chapple. 1997. Cinnamate-4-Hydroxylase Expression in Arabidopsis (Regulation in Response to Development and the Environment). Plant Physiol. 113:729-738.
Board, N. B. 2011. BioDiesel.
Bohlmann, J., and C. I. Keeling. 2008. Terpenoid biomaterials. Plant J. 54:656-669.
Bohlmann, J., Meyer-Gauen, G., Croteau, R. 1998. Plant terpenoid synthases: molecular biology and phylogenetic analysis. P Natl Acad Sci USA. 95:4126-4133.
Bonner, J. 1943. Effects of temperature on rubber accumulation by the Guayule plant. Bot Gaz. 105:233-243.
Brijwani, K., H. S. Oberoi, and P. V. Vadlani. 2010. Production of a cellulolytic enzyme system in mixed-culture solid-state fermentation of soybean hulls supplemented with wheat bran. Process Biochemistry. 45:120-128.
Callis, J., M. Fromm, and V. Walbot. 1987. Introns increase gene expression in cultured maize cells. Genes Dev. 1:1183-1200.
Carlson, S., G. Rudgers, H. Zieler, J. Mach, S. Luo, E. Grunden, C. Krol, G. Copenhaver, and D. Preuss. 2007. Meiotic transmission of an in vitro-assembled autonomous maize minichromosome. PLoS Genet. 3:1965-1974.
Cavaliere, F. M., G. L. Scoarughi, and C. Cimmino. 2009. Interspecific transfer of mammalian artificial chromosomes between farm animals. Chromosome Res. 17:507-517.
Cheng, A. X., Y. G. Lou, Y. B. Mao, S. Lu, L. J. Wang, and X. Y. Chen. 2007. Plant terpenoids: Biosynthesis and ecological functions. J Integr Plant Biol. 49:179-186.
Coffelt, T. A., F. S. Nakayama, D. T. Ray, K. Cornish, and C. M. McMahan. 2009a. Post-harvest storage effects on guayule latex, rubber, and resin contents and yields. Ind Crop Prod. 29:326-335.
Coffelt, T. A., F. S. Nakayama, D. T. Ray, K. Cornish, C. M. McMahan, and C. F. Williams. 2009b. Plant population, planting date, and germplasm effects on guayule latex, rubber, and resin yields. Ind Crop Prod. 29:255-260.
Conesa, A., S. Gotz, J. M. Garcia-Gomez, J. Terol, M. Talon, and M. Robles. 2005. Blast2GO: a universal tool for annotation, visualization and analysis in functional genomics research. Bioinformatics. 21:3674-3676.
Connor, M. R., and S. Atsumi. 2010. Synthetic biology guides biofuel production. J Biomed Biotechnol. 2010.
Cornish, K., and R. A. Backhaus. 2003. Induction of rubber transferase activity in guayule (Parthenium argentatum Gray) by low temperatures. Ind Crop Prod. 17:83-92.
Cornish, K., M. H. Chapman, J. L. Brichta, and D. J. Scott. 2000a. Effect of postharvest conditions on the yield of hypoallergenic latex from guayule (Parthenium argentatum Gray). Abstr Pap Am Chem S. 219:U191-U191.
Cornish, K., M. H. Chapman, J. L. Brichta, S. H. Vinyard, and F. S. Nakayama. 2000b. Post-harvest stability of latex in different sizes of guayule branches. Ind Crop Prod. 12:25-32.
Cornish, K., M. D. Myers, and S. S. Kelley. 2004. Latex quantification in homogenate and purified latex samples from various plant species using near infrared reflectance spectroscopy. Ind Crop Prod. 19:283-296.
Cornish, K., Myers, M. D. and Kelley, S. S. 2004. Quantification of rubber latex in homogenate and purified samples using near infrared spectroscopy. Industrial Crops and Products 19:283-296.
Crock J, W. M., Croteau R. 1997. Isolation and bacterial expression of a sesquiterpene synthase cDNA clone from peppermint (Mentha×piperita, L.) that produces the aphid alarm pheromone (E)-beta-farnesene. Proc Natl Acad Sci USA. 94:12833-12838.
Cunillera, N., M. Arro, D. Delourme, F. Karst, A. Boronat, and A. Ferrer. 1996. Arabidopsis thaliana contains two differentially expressed farnesyl-diphosphate synthase genes. J Biol Chem. 271:7774-7780.
Demyttenaere, J. C. R., R. M. Morina, N. De Kimpe, and P. Sandra. 2004. Use of headspace solid-phase microextraction and headspace sorptive extraction for the detection of the volatile metabolites produced by toxigenic Fusarium species. Journal of Chromatography a. 1027:147-154.
Dierig, D. A., D. T. Ray, T. A. Coffelt, F. S. Nakayama, G. S. Leake, and G. Lorenz. 2001. Heritability of height, width, resin, rubber, and latex in guayule (Parthenium argentatum). Ind Crop Prod. 13:229-238.
Dierig, D. T., A E; Ray, D T. 1996. Yield evaluation of new Arizona guayule selections. In New Industrial Crops and Products. A. T. Estilai, J P; Naqvi, H H, editor. Office of Arid Land Studies, University of Arizona, Tucson, Ariz.
Dunwell, J. M. 1999. Transformation of maize using silicon carbide whiskers. Methods in molecular biology (Clifton, N.J. 111:375-382.
Edris, A. E., R. Chizzola, and C. Franz. 2008. Isolation and characterization of the volatile aroma compounds from the concrete headspace and the absolute of Jasminum sambac (L.) Ait. (Oleaceae) flowers grown in Egypt. European Food Research and Technology. 226:621-626.
Enjuto, M., L. Balcells, N. Campos, C. Caelles, M. Arro, and A. Boronat. 1994. Arabidopsis-Thaliana Contains 2 Differentially Expressed 3-Hydroxy-3-Methylglutaryl-Coa Reductase Genes, Which Encode Microsomal Forms of the Enzyme. P Natl Acad Sci USA. 91:927-931.
Estevez, J. M., A. Cantero, C. Romero, H. Kawaide, L. F. Jimenez, T. Kuzuyama, H. Seto, Y. Kamiya, and P. Leon. 2000. Analysis of the expression of CLA1, a gene that encodes the 1-deoxyxylulose 5-phosphate synthase of the 2-C-methyl-D-erythritol-4-phosphate pathway in Arabidopsis. Plant Physiol. 124:95-103.
Estilai, A. 1985. Registration of Cal-5 Guayule Germplasm. Crop Sci. 25:369-370.
Estilai, A. 1986. Registration of Cal-6 and Cal-7 Guayule Germplasm. Crop Sci. 26:1261-1262.
Estilai, A. D., D. A. 1994. Improvement in rubber and resin yields of guayule through plant breeding. In Proc. of the Ninth Intl. Conf. on Jojoba and its Uses, and the Third Int. Conf. New Industrial Crops and Projects; September 25-30. L. R. Princen, C, editor, Catamarca, Argentina.
Fischer, C. R., D. Klein-Marcuschamer, and G. Stephanopoulos. 2008. Selection and optimization of microbial hosts for biofuels production. Metabolic Engineering. 10:295-304.
Gao, Z., X. Xie, Y. Ling, S. Muthukrishnan, and G. H. Liang. 2005. Agrobacterium tumefaciens-mediated sorghum transformation using a mannose selection system. Plant Biotechnology Journal. 3:591-599.
Gaxiola, R. A. L., J.; Undurraga, S.; Dang, L. M.; Allen, G. J.; Alper, S. L.; Fink, G. R. 2001. Drought- and salt-tolerant plants result from overexpression of the AVP1 H+-pump P Natl Acad Sci USA. 98:11444-11449.
Gounder, R., and E. Iglesia. 2011. Catalytic Alkylation Routes via Carbonium-Ion-Like Transition States on Acidic Zeolites. Chem Cat Chem. 3:1134-1138.
Greenhagen, B. T., P. E. O'Maille, J. P. Noel, and J. Chappell. 2006. Identifying and manipulating structural determinates linking catalytic specificities in terpene synthases. Proceedings of the National Academy of Sciences. 103:9826-9831.
Gurel, S., E. Gurel, R. Kaur, J. Wong, L. Meng, H.-Q. Tan, and P. Lemaux. 2009. Efficient, reproducible &lt;i&gt;Agrobacterium&lt;/i&gt;-mediated transformation of sorghum using heat treatment of immature embryos. Plant Cell Reports. 28:429-444.
Hall, A. E., A. Fiebig, and D. Preuss. 2002. Beyond the Arabidopsis genome: opportunities for comparative genomics. Plant Physiol. 129:1439-1447.
Hammond, B., Polhamus, L G. 1965. Research on guayule (Parthenium argentatum): 1942-1959. Vol. Technical Bulletin 1327. USDA-ARS, editor. 157.
Hernanz, D., V. Gallo, A. F. Recamales, A. J. Melendez-Martinez, and F. J. Heredia. 2008. Comparison of the effectiveness of solid-phase and ultrasound-mediated liquid-liquid extractions to determine the volatile compounds of wine. Talanta. 76:929-935.
Huber D P, P. R., Godard K A, Sturrock R N, Bohlmann J. 2005. Characterization of four terpene synthase cDNAs from methyl jasmonate-induced Douglas-fir, Pseudotsuga menziesii. Phytochemistry. 66:1427-1439.
Knapik, A., A. Drelinkiewicz, A. Waksmundzka-Góra, A. Bukowska, W. Bukowski, and J. Noworól. 2008. Hydrogenation of 2-Butyn-1,4-diol in the Presence of Functional Crosslinked Resin Supported Pd Catalyst. The Role of Polymer Properties in Activity/Selectivity Pattern. Catalysis Letters. 122:155-166.
   Kóller, T. G., J. Gershenzon, and J. Degenhardt. 2009. Molecular and biochemical evolution of maize terpene synthase 10, an enzyme of indirect defense. Phytochemistry. 70:1139-1145.
Kumar, S., Hahn, F. M., McMahan, C. M., Cornish, K., Whalen, M. C. 2009. Comparative analysis of the complete sequence of the plastid genome of Parthenium argentatum and identification of DNA barcodes to differentiate Parthenium species and lines. BMC Plant Biology. 9:: 131.
Lai, S. M., I. W. Chen, and M. J. Tsai. 2005. Preparative isolation of terpene trilactones from Ginkgo biloba leaves. Journal of Chromatography a. 1092:125-134.
LEWINSOHN, E., N. DUDAI, Y. TADMOR, I. KATZIR, U. RAVID, E. PUTIEVSKY, and D. M. JOEL. 1998. Histochemical Localization of Citral Accumulation in Lemongrass Leaves (Cymbopogon citratus(DC.) Stapf., Poaceae). Annals of Botany. 81:35-39.
Li, J. S., H. B. Yang, W. A. Peer, G. Richter, J. Blakeslee, A. Bandyopadhyay, B. Titapiwantakun, S. Undurraga, M. Khodakovskaya, E. L. Richards, B. Krizek, A. S. Murphy, S. Gilroy, and R. Gaxiola. 2005. Arabidopsis H+-PPase AVP1 regulates auxin-mediated organ development. Science. 310:121-125.
Liang, X. W., M. Dron, C. L. Cramer, R. A. Dixon, and C. J. Lamb. 1989. Differential regulation of phenylalanine ammonia-lyase genes during plant development and by environmental cues. J Biol Chem. 264:14486-14492.
Lin, Y., and S. Tanaka. 2006. Ethanol fermentation from biomass resources: current state and prospects. Appl Microbiol Biotechnol. 69:627-642.
Martin, J., V. M. Bruno, Z. Fang, X. Meng, M. Blow, T. Zhang, G. Sherlock, M. Snyder, and Z. Wang. 2010. Rnnotator: an automated de novo transcriptome assembly pipeline from stranded RNA-Seq reads. BMC Genomics. 11:663.
Maruyama T, I. M., Honda G. 2001. Molecular cloning, functional expression and characterization of (E)-beta farnesene synthase from Citrus junos. Biol Pharm Bull. 10:1171-1175.
Maury, S., P. Geoffroy, and M. Legrand. 1999. Tobacco O-Methyltransferases Involved in Phenylpropanoid Metabolism. The Different Caffeoyl-Coenzyme A/5-Hydroxyferuloyl-Coenzyme A 3/5-O-Methyltransferase and Caffeic Acid/5-Hydroxyferulic Acid 3/5-O-Methyltransferase Classes Have Distinct Substrate Specificities and Expression Patterns. Plant Physiol. 121:215-224.
McMahan, C. M., K. Cornish, T. A. Coffelt, F. S. Nakayama, R. G. McCoy, J. L. Brichta, and D. T. Ray. 2006. Post-harvest storage effects on guayule latex quality from agronomic trials. Ind Crop Prod. 24:321-328.
Mookdasanit, J., H. Tamura, T. Yoshizawa, T. Tokunaga, and K. Nakanishi. 2003. Trace volatile components in essential oil of Citrus sudachi by means of modified solvent extraction method. Food Science and Technology Research. 9:54-61.
Nair, R. B., Q. Xia, C. J. Kartha, E. Kurylo, R. N. Hirji, R. Datla, and G. Selvaraj. 2002. Arabidopsis CYP98A3 Mediating Aromatic 3-Hydroxylation. Developmental Regulation of the Gene, and Expression in Yeast. Plant Physiol. 130:210-220.
Needleman, S. B., and C. D. Wunsch. 1970. A general method applicable to the search for similarities in the amino acid sequence of two proteins. Journal of molecular biology. 48:443-453.
Newell, R. 2011. Annual Energy Outlook 2011, Reference Case.
Niehaus, M. 1983. The role of Guayule Admin. Manag. Comm. In guayule commercialization/research. El Guayulero. 5:15-19.
Nigam, P. S., and A. Singh. 2011. Production of liquid biofuels from renewable resources. Progress in Energy and Combustion Science. 37:52-68.
Oberoi, H. S., P. V. Vadlani, R. L. Madl, L. Saida, and J. P. Abeykoon. 2010. Ethanol Production from Orange Peels: Two-Stage Hydrolysis and Fermentation Studies Using Optimized Parameters through Experimental Design. Journal of Agricultural and Food Chemistry. 58:3422-3429.
Pearson, C. H., K. Cornish, C. M. McMahan, D. J. Rath, and M. Whalen. 2010. Natural rubber quantification in sunflower using an automated solvent extractor. Ind Crop Prod. 31:469-475.
Pechous, S. W., C. B. Watkins, and B. D. Whitaker. 2005. Expression of alpha-farnesene synthase gene AFS1 in relation to levels of alpha-farnesene and conjugated trienols in peel tissue of scald-susceptible ‘Law Rome’ and scald-resistant ‘Idared’ apple fruit. Postharvest Biology and Technology. 35:125-132.
Peralta-Yahya, P., and J. Keasling. 2010. Advanced biofuel production in microbes. Biotechnol J. 5:147-162.
Petrasovits, L. A. P., M. P.; Nielsen, L. K.; Brumbley, S. M. 2007. Production of polyhydroxybutyrate in sugarcane. Plant Biotechnology Journal. 5:162-172.
Picaud S, B. M., Brodelius P E. 2005. Expression, purification and characterization of recombinant (E)-beta-farnesene synthase from Artemisia annua. Phytochemistry. 66:961-967.
Pourbafrani, M., G. Forgacs, I. S. Horvath, C. Niklasson, and M. J. Taherzadeh. 2010. Production of biofuels, limonene and pectin from citrus wastes. Bioresour Technol. 101:4246-4250.
Ray, D. T., D. A. Dierig, A. E. Thompson, and T. A. Coffelt. 1999. Registration of six guayule germplasms with high yielding ability. Crop Sci. 39:300-300.
Reed, J., L. Privalle, M. Powell, M. Meghji, J. Dawson, E. Dunder, J. Sutthe, A. Wenck, K. Launis, C. Kramer, Y.-F. Chang, G. Hansen, and M. Wright. 2001. Phosphomannose isomerase: An efficient selectable marker for plant transformation. In Vitro Cellular &amp; Developmental Biology-Plant. 37:127-132.
RFA. 2011. Renewable Fuels Association-ethanol facts.
Rout, P. K., S. N. Naika, and Y. R. Rao. 2008. Subcritical CO2 extraction of floral fragrance from Quisqualis indica. Journal of Supercritical Fluids. 45:200-205.
Sakakibara, Y. K., H.; Kasamo, K. 1996. Isolation and characterization of cDNAs encoding vacuolar H⁺-pyrophosphatase isoforms from rice (Oryza sativa L.). Plant Molecular Biology. 31:1029-1038.
Salvucci, M. E., T. A. Coffelt, and K. Cornish. 2009. Improved methods for extraction and quantification of resin and rubber from guayule. Ind Crop Prod. 30:9-16.
Schnee, C., T. G. Kollner, M. Held, T. C. J. Turlings, J. Gershenzon, and J. Degenhardt. 2006. The products of a single maize sesquiterpene synthase form a volatile defense signal that attracts natural enemies of maize herbivores. P Natl Acad Sci USA. 103:1129-1134.
Serrano, A., and M. Gallego. 2006. Continuous microwave-assisted extraction coupled on-line with liquid-liquid extraction: Determination of aliphatic hydrocarbons in soil and sediments. Journal of Chromatography a. 1104:323-330.
Tholl, D. 2006. Terpene synthases and the regulation, diversity and biological roles of terpene metabolism. Current Opinion in Plant Biology. 9:1-8.
Tipton, J. L., and E. C. Gregg. 1982. Variation in Rubber Concentration of Native Texas Guayule. Hortscience. 17:742-743.
Tysdal, H. M., A. Estilai, I. A. Siddiqui, and P. F. Knowles. 1983. Registration of 4 Guayule Germplasms. Crop Sci. 23:189-189.
Unger, E. A., J. M. Hand, A. R. Cashmore, and A. C. Vasconcelos. 1989. Isolation of a cDNA encoding mitochondrial citrate synthase from Arabidopsis thaliana. Plant Mol Biol. 13:411-418.
Van den Broeck, G., Timko, M. P., Kausch, A. P., Cashmore, A. R., Van Montagu, M, Herrera-Estrella, L. 1985. Targeting of a foreign peptide to chloroplasts by fusion to the transit peptide from the small subunit of ribulose 1,5-bisphosphate carboxylase. Nature. 313:358-363.
Veatch, M. E., D. T. Ray, C. J. D. Mau, and K. Cornish. 2005. Growth, rubber, and resin evaluation of two-year-old transgenic guayule. Ind Crop Prod. 22:65-74.
von Heijne, G., Steppuhn, J., Herrmann, R. G. 1989. Domain structure of mitochondrial and chloroplast targeting peptides. European Journal of Biochemistry. 180:535-545.
Whitworth, J. W., EE. 1991. Guayule natural rubber: a technical publication with emphasis on recent findings. USDA-ARS, editor. Office of Arid Land Studies, The University of Arizona, Tucson. 445.
Wienk, H. L. J., Wechselberger, R. W., Czisch, M., de Kruijff, B. 2000. Structure, Dynamics, and Insertion of a Chloroplast Targeting Peptide in Mixed Micelles. Biochemistry. 39:8219-8227.
Wu, S., M. Schalk, A. Clark, R. B. Miles, R. Coates, and J. Chappell. 2006. Redirection of cytosolic or plastidic isoprenoid precursors elevates terpene production in plants. Nat Biotechnol. 24:1441-1447.
Yoshikuni, Y., and B.w.t.U.o.C. University of California, San Francisco. 2007. Redesigning enzymes based on the theories of molecular evolution for optimal function in synthetic metabolic pathways. University of California, Berkeley with the University of California, San Francisco.
Zhan, X., D. Wang, M. R. Tuinstra, S. Bean, P. A. Seib, and X. S. Sun. 2003. Ethanol and lactic acid production as affected by sorghum genotype and location. Ind Crop Prod. 18:245-255.
Zhang, J., X.-Z. Sun, M. Poliakoff, and M. W. George. 2003. Study of the reaction of Rh(acac)(C0)2 with alkenes in polyethylene films under high-pressure hydrogen and the Rh-catalysed hydrogenation of alkenes. Journal of Organometallic Chemistry. 678:128-133.
Zhao, Z.-y. 2006. Sorghum (&lt;i&gt;Sorghum bicolor&lt;/i&gt; L.). ln &lt;i&gt;Agrobacterium&lt;/i&gt; Protocols. Vol. 343. K. Wang, editor. Humana Press. 233-244.
Zheng, C. H., T. H. Kim, K. H. Kim, Y. H. Leem, and H. J. Lee. 2004. Characterization of potent aroma compounds in Chrysanthemum coronarium L. (Garland) using aroma extract dilution analysis. Flavour and Fragrance Journal. 19:401-405.
Zini, C. A., K. D. Zanin, E. Christensen, E. B. Caramao, and J. Pawliszyn. 2003. Solid-phase microextraction of volatile compounds from the chopped leaves of three species of Eucalyptus. Journal of Agricultural and Food Chemistry. 51:2679-2686.
Zuo, J., Q. W. Niu, G. Frugis, and N. H. Chua. 2002. The WUSCHEL gene promotes vegetative-to-embryonic transition in Arabidopsis. Plant J. 30:349-359.

We claim:

1. A plant cell having increased production of at least one terpenoid native to a plant, the method comprising expressing in a plant cell a heterologous nucleic acid encoding for (a) HMG-CoA reductase, (b) 1-deoxy-D-xylulose-5-phosphate synthase, (c) farnesyl pyrophosphate synthase, and (d) β-farnesene synthase, wherein production of the at least one terpenoid is significantly increased when compared to a wild-type plant cell not encoding the heterologous nucleic acids.

2. The method of claim 1, wherein
a. the HMG-CoA reductase is an Arabidopsis, Oryza, Saccharomyces, or Hevea HMG-CoA reductase;
b. the 1-deoxy-D-xyululose-5-phophate is an Arabidopsis, Oryza, Saccharomyces, or Zea 1-deoxy-D-xyululose;
c. the farnesyl pyrophosphate synthase is an Arabidopsis, Oryza, or Solanum farnesyl pyrophosphate; or
d. the β-farnesene synthase is an Arabidopsis, Oryza, or Artemisia β-farnesene synthase.

3. The method of claim 2, wherein
a. the HMG-CoA reductase is an Arabidopsis thaliana, Oryza sativa, Saccharomyces cerevisiae, or Hevea HMG-CoA reductase;
b. the 1-deoxy-D-xyululose-5-phophate is an Arabidopsis thaliana, Oryza sativa, Saccharomyces cerevisiae, or Zea mays 1-deoxy-D-xyululose;
c. the farnesyl pyrophosphate synthase is an Arabidopsis thaliana, Oryza sativa, or Solanum lycopersicon farnesyl pyrophosphate;
d. the β-farnesene synthase is an Arabidopsis thaliana, Oryza sativa, or Artemisia annua β-farnesene synthase.

4. The method of claim 3, wherein at least one nucleic acid is codon-optimized for expression in a plant.

5. The method of claim 3, wherein
a. the HMG-CoA reductase is encoded by a polynucleotide having at least 70% sequence identity to a nucleic acid sequence selected from the group consisting of SEQ ID NOs:1, 2, 3, 16, 17, and 28;
b. the 1-deoxy-D-xyululose-5-phophate is encoded by a polynucleotide having at least 70% sequence identity to a nucleic acid sequence selected from the group consisting of SEQ ID NOs: 4, 5, 6, 18, 19 and 20;
c. the farnesyl pyrophosphate synthase is encoded by a polynucleotide having at least 70% sequence identity to a nucleic acid sequence selected from the group consisting of SEQ ID NOs:7, 8, 9, 21, 22, 23, 24 and 29;
d. an AVP1/OMP1 is encoded by a polynucleotide having at least 70% sequence identity to a nucleic acid sequence selected from the group consisting of SEQ ID NOs:13, 14, 15, and 27; or
e. the β-farnesene synthase is encoded by a polynucleotide having at least 70% sequence identity to a nucleic acid sequence selected from the group consisting of SEQ ID NOs:10, 11, 12, 25, and 26.

6. The method of claim 3, wherein
a. an HMG-CoA reductase is encoded by a polynucleotide having at a nucleic acid sequence of SEQ ID NOs:1, 2, 3, 16, 17, or 28;
b. a 1-deoxy-D-xyululose-5-phophate is encoded by a polynucleotide having at a nucleic acid sequence of SEQ ID NOs:4, 5, 6, 18, 19, or 20;
c. a farnesyl pyrophosphate synthase is encoded by a polynucleotide having at a nucleic acid sequence selected from the group consisting of SEQ ID NOs:7, 8, 9, 21, 22, 23, 24, or 29;
d. an AVP1/OMP1 is encoded by a polynucleotide having at a nucleic acid sequence selected from the group consisting of SEQ ID NOs:13, 14, 15, or 27; or
e. a β-farnesene synthase is encoded by a polynucleotide having at a nucleic acid sequence selected from the group consisting of SEQ ID NOs:10, 11, 12, 25 or 26.

7. The method of claim 5, wherein the heterologous polynucleotide comprises a nucleic acid sequence encoding an FVE or a GWD gene.

8. The method of claim 1, wherein the plant cell comprises HMG-CoA reductase, farnesyl pyrophosphate synthase, β-farnesene synthase and AVP1/OMP1 heterologous nucleic acids.

9. The method of claim 8, wherein the nucleic acids are operably linked to constitutive promoters.

10. The method of claim 1, wherein the plant cell comprises HMG-CoA reductase, farnesyl pyrophosphate synthase, and β-farnesene synthase heterologous nucleic acids.

11. The method of claim 10, wherein the nucleic acids are operably linked to a tissue-specific or developmental-specific promoter.

12. The method of claim 11, wherein the promoter is a lignin promoter.

13. The method of claim 1, wherein the plant cell comprises 1-deoxy-D-xylulose-5-phosphate synthase, farnesyl pyrophosphate synthase and β-farnesene synthase heterologous nucleic acids.

14. The method of claim 13, wherein the polypeptides encoded by the heterologous nucleic acids are targeted to a chloroplast of the plant cell.

15. The method of claim 1, wherein the plant cell is a cell from a plant selected from the group consisting of a green algae, a vegetable crop plant, a fruit crop plant, a vine crop plant, a field crop plant, a biomass plant, a bedding plant, and a tree.

16. The method of claim 15, wherein the plant is selected from the group consisting of corn, soybean, Brassica, tomato, sorghum, sugarcane, guayule, miscanthus, switchgrass, wheat, barley, oat, rye, wheat, rice, beet, green algae and cotton.

17. The method of claim 15, wherein the plant is sorghum, sugarcane, or guayule.

18. The method of claim 17, wherein the plant cell is a guayule plant cell, and the cell expresses:
a. an HMG-CoA reductase is encoded by a polynucleotide having at least 70% sequence identity to a nucleic acid sequence of SEQ ID NOs:1, 2, 3, 16, 17, or 28;
b. a 1-deoxy-D-xyululose-5-phophate is encoded by a polynucleotide having at least 70% sequence identity to a nucleic acid sequence of SEQ ID NOs:4, 5, 6, 18, 19, or 20;
c. a farnesyl pyrophosphate synthase is encoded by a polynucleotide having at least 70% sequence identity to a nucleic acid sequence selected from the group consisting of SEQ ID NOs:7, 8, 9, 21, 22, 23, 24, or 29;
d. an AVP1/OMP1 is encoded by a polynucleotide having at least 70% sequence identity to a nucleic acid sequence selected from the group consisting of SEQ ID NOs:13, 14, 15, or 27; or
e. a β-farnesene synthase is encoded by a polynucleotide having at least 70% sequence identity to a nucleic acid sequence selected from the group consisting of SEQ ID NOs:10, 11, 12, 25 or 26.

19. The method of claim 17, wherein the plant cell is a guayule plant cell, and the cell expresses:
a. an HMG-CoA reductase is encoded by a polynucleotide having a nucleic acid sequence of SEQ ID NOs:1, 2, 3, 16, 17, or 28;
b. a 1-deoxy-D-xyululose-5-phophate is encoded by a polynucleotide having a nucleic acid sequence of SEQ ID NOs:4, 5, 6, 18, 19, or 20;
c. a farnesyl pyrophosphate synthase is encoded by a polynucleotide having a nucleic acid sequence selected from the group consisting of SEQ ID NOs:7, 8, 9, 21, 22, 23, 24, or 29;
d. an AVP1/OMP1 is encoded by a polynucleotide having a nucleic acid sequence selected from the group consisting of SEQ ID NOs:13, 14, 15, or 27; or
e. a β-farnesene synthase is encoded by a polynucleotide having a nucleic acid sequence selected from the group consisting of SEQ ID NOs:10, 11, 12, 25 or 26.

20. The method of claim 17, wherein the plant cell is a sorghum plant cell, and the cell expresses:
a. an HMG-CoA reductase is encoded by a polynucleotide having at least 70% sequence identity to a nucleic acid sequence of SEQ ID NOs:1, 2, 3, 16, 17, or 28;
b. a 1-deoxy-D-xyululose-5-phophate is encoded by a polynucleotide having at least 70% sequence identity to a nucleic acid sequence of SEQ ID NOs:4, 5, 6, 18, 19, or 20;
c. a farnesyl pyrophosphate synthase is encoded by a polynucleotide having at least 70% sequence identity to a nucleic acid sequence selected from the group consisting of SEQ ID NOs:7, 8, 9, 21, 22, 23, 24, or 29;
d. an AVP1/OMP1 is encoded by a polynucleotide having at least 70% sequence identity to a nucleic acid sequence selected from the group consisting of SEQ ID NOs:13, 14, 15, or 27; or
e. a β-farnesene synthase is encoded by a polynucleotide having at least 70% sequence identity to a nucleic acid sequence selected from the group consisting of SEQ ID NOs:10, 11, 12, 25 or 26.

21. The method of claim 20, wherein the plant cell is a sorghum plant cell, and the cell expresses:
a. an HMG-CoA reductase is encoded by a polynucleotide having a nucleic acid sequence of SEQ ID NOs:1, 2, 3, 16, 17, or 28;
b. a 1-deoxy-D-xyululose-5-phophate is encoded by a polynucleotide having a nucleic acid sequence of SEQ ID NOs:4, 5, 6, 18, 19, or 20;
c. a farnesyl pyrophosphate synthase is encoded by a polynucleotide having a nucleic acid sequence selected from the group consisting of SEQ ID NOs:7, 8, 9, 21, 22, 23, 24, or 29;
d. an AVP1/OMP1 is encoded by a polynucleotide having a nucleic acid sequence selected from the group consisting of SEQ ID NOs:13, 14, 15, or 27; or
e. a β-farnesene synthase is encoded by a polynucleotide having a nucleic acid sequence selected from the group consisting of SEQ ID NOs:10, 11, 12, 25 or 26.

22. The method of claim 17, wherein the plant cell is a sugarcane plant cell, and the cell expresses:
a. an HMG-CoA reductase is encoded by a polynucleotide having at least 70% sequence identity to a nucleic acid sequence of SEQ ID NOs:1, 2, 3, 16, 17, or 28;
b. a 1-deoxy-D-xyululose-5-phophate is encoded by a polynucleotide having at least 70% sequence identity to a nucleic acid sequence of SEQ ID NOs:4, 5, 6, 18, 19, or 20;
c. a farnesyl pyrophosphate synthase is encoded by a polynucleotide having at least 70% sequence identity to a nucleic acid sequence selected from the group consisting of SEQ ID NOs:7, 8, 9, 21, 22, 23, 24, or 29;
d. an AVP1/OMP1 is encoded by a polynucleotide having at least 70% sequence identity to a nucleic acid sequence selected from the group consisting of SEQ ID NOs:13, 14, 15, or 27; or
e. a β-farnesene synthase is encoded by a polynucleotide having at least 70% sequence identity to a nucleic acid sequence selected from the group consisting of SEQ ID NOs:10, 11, 12, 25 or 26.

23. The method of claim 20, wherein the plant cell is a sugarcane plant cell, and the cell expresses:
a. a an HMG-CoA reductase is encoded by a polynucleotide having a nucleic acid sequence of SEQ ID NOs:1, 2, 3, 16, 17, or 28;
b. a 1-deoxy-D-xyululose-5-phophate is encoded by a polynucleotide having a nucleic acid sequence of SEQ ID NOs:4, 5, 6, 18, 19, or 20;
c. a farnesyl pyrophosphate synthase is encoded by a polynucleotide having a nucleic acid sequence selected from the group consisting of SEQ ID NOs:7, 8, 9, 21, 22, 23, 24, or 29;
d. an AVP1/OMP1 is encoded by a polynucleotide having a nucleic acid sequence selected from the group consisting of SEQ ID NOs:13, 14, 15, or 27; or
e. a β-farnesene synthase is encoded by a polynucleotide having a nucleic acid sequence selected from the group consisting of SEQ ID NOs:10, 11, 12, 25 or 26.

24. The method of claim 1, wherein the at least one terpenoid is a sesquiterpenoid.

25. The method of claim 24, wherein the sesquiterpenoid is farnesene.

26. The method of claim 1, wherein at least one heterologous nucleic acid is operably linked to a constitutive promoter.

27. The method of claim 1, wherein at least on heterologous nucleic acid is operably linked to an inducible or tissue-specific promoter.

28. The method of claim 1, wherein an autonomous DNA construct in the plant cell comprises at least one heterologous nucleic acid.

29. The method of claim 28, wherein the autonomous DNA construct is a mini-chromosome.

30. The method of claim 29, wherein the mini-chromosome comprises a centromere derived from the species of the plant cell.

31. The method of claim 1, further comprising isolating the farnesene.

32. The method of claim 31, wherein the isolated farnesene is further processed into farnesane.

33. A plant cell comprising heterologous nucleic acids derived from a plant and encoding for (a) HMG-CoA reductase, (b) 1-deoxy-D-xylulose-5-phosphate synthase, (c) farnesyl pyrophosphate synthase, and (d) β-farnesene synthase, wherein production of at least one terpenoid is significantly increased when compared to a wild-type plant cell not expressing the heterologous nucleic acids.

34. The plant cell of claim 33, wherein
a. the HMG-CoA reductase is an Arabidopsis, Oryza, Saccharomyces or Hevea HMG-CoA reductase;
b. the 1-deoxy-D-xyululose-5-phophate is an Arabidopsis, Oryza, Saccharomyces, or Zea 1-deoxy-D-xyululose;
c. the farnesyl pyrophosphate synthase is an Arabidopsis, Oryza, or Solanum farnesyl pyrophosphate;
d. the AVP1/OMP1 is an Arabidopsis, Oryza, or Triticum AVP1/OMP1; or
e. the β-farnesene synthase is an Arabidopsis, Oryza, or Artemisia β-farnesene synthase.

35. The plant cell of claim 34, wherein
a. the HMG-CoA reductase is an Arabidopsis thaliana, Oryza sativa, Saccharomyces cerevisiae or Hevea HMG-CoA reductase;
b. the 1-deoxy-D-xyululose-5-phophate is an Arabidopsis thaliana, Oryza sativa, Saccharomyces cerevisiae or Zea mays 1-deoxy-D-xyululose;
c. the farnesyl pyrophosphate synthase is an Arabidopsis thaliana, Oryza sativa, or Solanum lycopersicon farnesyl pyrophosphate;
d. the AVP1/OMP1 is an Arabidopsis thaliana, Oryza sativa, or Triticum aestivum AVP1/OMP1; or
e. the β-farnesene synthase is an Arabidopsis thaliana, Oryza sativa, or Artemisia annua β-farnesene synthase.

36. The plant cell of claim 35, wherein
a. an HMG-CoA reductase is encoded by a polynucleotide having at least 70% sequence identity to a nucleic acid sequence of SEQ ID NOs:1, 2, 3, 16, 17, or 28;
b. a 1-deoxy-D-xyululose-5-phophate is encoded by a polynucleotide having at least 70% sequence identity to a nucleic acid sequence of SEQ ID NOs:4, 5, 6, 18, 19, or 20;
c. a farnesyl pyrophosphate synthase is encoded by a polynucleotide having at least 70% sequence identity to a nucleic acid sequence selected from the group consisting of SEQ ID NOs:7, 8, 9, 21, 22, 23, 24, or 29;
d. an AVP1/OMP1 is encoded by a polynucleotide having at least 70% sequence identity to a nucleic acid sequence selected from the group consisting of SEQ ID NOs:13, 14, 15, or 27; or
e. a β-farnesene synthase is encoded by a polynucleotide having at least 70% sequence identity to a nucleic acid sequence selected from the group consisting of SEQ ID NOs:10, 11, 12, 25 or 26.

37. The plant cell of claim 36, wherein
a. an HMG-CoA reductase is encoded by a polynucleotide having a nucleic acid sequence of SEQ ID NOs:1, 2, 3, 16, 17, or 28;
b. a 1-deoxy-D-xyululose-5-phophate is encoded by a polynucleotide having a nucleic acid sequence of SEQ ID NOs:4, 5, 6, 18, 19, or 20;
c. a farnesyl pyrophosphate synthase is encoded by a polynucleotide having a nucleic acid sequence selected from the group consisting of SEQ ID NOs:7, 8, 9, 21, 22, 23, 24, or 29;
d. an AVP1/OMP1 is encoded by a polynucleotide having a nucleic acid sequence selected from the group consisting of SEQ ID NOs:13, 14, 15, or 27; or
e. a β-farnesene synthase is encoded by a polynucleotide having a nucleic acid sequence selected from the group consisting of SEQ ID NOs:10, 11, 12, 25 or 26.

38. The plant cell of claim 33, wherein the plant cell comprises HMG-CoA reductase, farnesyl pyrophosphate synthase, β-farnesene synthase and AVP1/OMP1 heterologous nucleic acids.

39. The method of claim 38, wherein the nucleic acids are operably linked to constitutive promoters.

40. The method of claim 33, wherein the plant cell comprises HMG-CoA reductase, farnesyl pyrophosphate synthase, and β-farnesene synthase heterologous nucleic acids.

41. The method of claim 40, wherein the nucleic acids are operably linked to a tissue-specific or developmental-specific promoter.

42. The method of claim 41, wherein the promoter is a lignin promoter.

43. The method of claim 33, wherein the plant cell comprises 1-deoxy-D-xylulose-5-phosphate synthase, farnesyl pyrophosphate synthase and β-farnesene synthase heterologous nucleic acids.

44. The method of claim 43, wherein the polypeptides encoded by the heterologous nucleic acids are targeted to a chloroplast of the plant cell.

45. The plant cell of claim 33, wherein the plant cell is a cell from a plant selected from the group consisting of a green algae, a vegetable crop plant, a fruit crop plant, a vine crop plant, a field crop plant, a biomass plant, a bedding plant, and a tree.

46. The plant cell of claim 38, wherein the plant is selected from the group consisting of corn, soybean, Brassica, tomato, sorghum, sugarcane, guayule, miscanthus, switchgrass, wheat, barley, oat, rye, wheat, rice, beet, green algae and cotton.

47. The plant cell of claim 46, wherein the plant is sorghum, sugarcane, or guayule.

48. The plant cell of claim 47, wherein the plant is sorghum, and the sorghum is sweet sorghum.

49. The plant cell of claim 33, wherein the at least one terpenoid is a sesquiterpenoid.

50. The plant cell of claim 49, wherein the sesquiterpenoid is farnesene.

51. The plant cell of claim 33, wherein at least one heterologous nucleic acid is operably linked to a constitutive promoter.

52. The plant cell of claim 33, wherein at least on heterologous nucleic acid is operably linked to an inducible or tissue-specific promoter.

53. The plant cell of claim 33, wherein an autonomous DNA construct in the plant cell comprises at least one heterologous nucleic acid.

54. The plant cell of claim 53, wherein the autonomous DNA construct is a mini-chromosome.

55. The plant cell of claim 54, wherein the mini-chromosome comprises a centromere derived from the species of the plant cell.

56. A fuel comprising a terpenoid made according to any of claims 1-32, or made by a plant cell of any of claims 33-55.

57. The fuel of claim 56, wherein the terpenoid is a sesquiterpenoid.

58. The fuel of claim 57, wherein the sesquiterpenoid is farnesene.


SEQUENCE LISTING (29 sequences)

SEQ ID NO: 1 - protein, 91 aa, Arabidopsis thaliana
MPSIEVGTVGGGTQLASQSACLNLLGVKGASTESPGMNARRLATIVAGAVLAGELSLMSAIAAGQLVRSHMKYNRSSRDISGATTTTTTTT

SEQ ID NO: 2 - protein, 576 aa, Oryza sativa
MAVEGRRRVPLPLPPPTRRGKQQQQQGGERARRVQAGDALPLPIRHTNLIFSALFAASLAYLMRRWREKIRTSTPLHVVGLAEILAICGLVASLIYLLSFFGIAFVQSVVSNSDDEEEEEDFLIDSRAAGPVAAQATPPPAPAPFSLLGSACAAPKKMPEEDEEIVAEVVAGKIPSYVLETRLGDCRRAAGIRREALRRTTGREIRGLPLDGFDYASILGQCCELPVGYVQLPVGVAGPLVLDGERFYVPMATTEGCLVASTNRGCKAIAESGGATSVVLQDGMTRAPVARFPSARRAAELKGFLENPANFDTLAMVFNRSSRFARLQRVKCAVAGRNLYMRFSCSTGDAMGMNMVSKGVQNVLDYLQDDFPDMDVISISGNFCSDKKSAAVNWIEGRGKSVVCEAVIKEEVVKKVLKTNVQSLVELNVIKNLAGSAVAGALGGFNAHASNIVTAIFIATGQDPAQNVESSQCITMLEAVNDGKDLHISVTMPSIEVGTVGGGTQLASQSACLDLLGVKGANRESPGSNARLLAAVVAGAVLAGELSLISAQAAGHLVQSHMKYNRSSKDMSKVAS

SEQ ID NO: 3 - protein, 575 aa, Hevea brasiliensis
MDTTGRLHHRKHATPVEDRSPTTPKASDALPLPLYLTNAVFFTLFFSVAYYLLHRWRDKIRNSTPLHIVTLSEIVAIVSLIASFIYLLGFFGIDFVQSFIARASHDVWDLEDTDPNYLIDEDHRLVTCPPANISTKTTIIAAPTKLPTSEPLIAPLVSEEDEMIVNSVVDGKIPSYSLESKLGDCKRAAAIRREALQRMTRRSLEGLPVEGFDYESILGQCCEMPVGYVQIPVGIAGPLLLNGREYSVPMATTEGCLVASTNRGCKAIYLSGGATSVLLKDGMTRAPVVRFASATRAAELKFFLEDPDNFDTLAVVFNKSSRFARLQGIKCSIAGKNLYIRFSYSTGDAMGMNMVSKGVQNVLEFLQSDFSDMDVIGISGNFCSDKKPAAVNWIEGRGKSVVCEAIIKEEVVKKVLKTNVASLVELNMLKNLAGSAVAGALGGFNAHAGNIVSAIFIATGQDPAQNVESSHCITMMEAVNDGKDLHISVTMPSIEVGTVGGGTQLASQSACLNLLGVKGANKESPGSNSRLLAAIVAGSVLAGELSLMSAIAAGQLVKSHMKYNRSSKDMSKAAS

SEQ ID NO: 4 - protein, 717 aa, Arabidopsis thaliana
MASSAFAFPSYIITKGGLSTDSCKSTSLSSSRSLVTDLPSPCLKPNNNSHSNRRAKVCASLAEKGEYYSNRPPTPLLDTINYPIHMKNLSVKELKQLSDELRSDVIFNVSKTGGHLGSSLGVVELTVALHYIFNTPQDKILWDVGHQSYPHKILTGRRGKMPTMRQTNGLSGFTKRGESEHDCFGTGHSSTTISAGLGMAVGRDLKGKNNNVVAVIGDGAMTAGQAYEAMNNAGYLDSDMIVILNDNKQVSLPTATLDGPSPPVGALSSALSRLQSNPALRELREVAKGMTKQIGGPMHQLAAKVDEYARGMISGTGSSLFEELGLYYIGPVDGHNIDDLVAILKEVKSTRTTGPVLIHVVTEKGRGYPYAERADDKYHGVVKFDPATGRQFKTTNKTQSYTTYFAEALVAEAEVDKDVVAIHAAMGGGTGLNLFQRRFPTRCFDVGIAEQHAVTFAAGLACEGLKPFCAIYSSFMQRAYDQVVHDVDLQKLPVRFAMDRAGLVGADGPTHCGAFDVTFMACLPNMIVMAPSDEADLFNMVATAVAIDDRPSCFRYPRGNGIGVALPPGNKGVPIEIGKGRILKEGERVALLGYGSAVQSCLGAAVMLEERGLNVTVADARFCKPLDRALIRSLAKSHEVLITVEEGSIGGFGSHVVQFLALDGLLDGKLKWRPMVLPDRYIDHGAPADQLAEAGLMPSHIAATALNLIGAPREALF

SEQ ID NO: 5 - protein, 720 aa, Oryza sativa
MALTTFSISRGGFVGALPQEGHFAPAAAELSLHKLQSRPHKARRRSSSSISASLSTEREAAEYHSQRPPTPLLDTVNYPIHMKNLSLKELQQLADELRSDVIFHVSKTGGHLGSSLGVVELTVALHYVFNTPQDKILWDVGHQSYPHKILTGRRDKMPTMRQTNGLSGFTKRSESEYDSFGTGHSSTTISAALGMAVGRDLKGGKNNVVAVIGDGAMTAGQAYEAMNNAGYLDSDMIVILNDNKQVSLPTATLDGPAPPVGALSSALSKLQSSRPLRELREVAKGVTKQIGGSVHELAAKVDEYARGMISGSGSTLFEELGLYYIGPVDGHNIDDLITILREVKSTKTTGPVLIHVVTEKGRGYPYAERAADKYHGVAKFDPATGKQFKSPAKTLSYTNYFAEALIAEAEQDNRVVAIHAAMGGGTGLNYFLRRFPNRCFDVGIAEQHAVTFAAGLACEGLKPFCAIYSSFLQRGYDQVVHDVDLQKLPVRFAMDRAGLVGADGPTHCGAFDVTYMACLPNMVVMAPSDEAELCHMVATAAAIDDRPSCFRYPRGNGIGVPLPPNYKGVPLEVGKGRVLLEGERVALLGYGSAVQYCLAAASLVERHGLKVTVADARFCKPLDQTLIRRLASSHEVLLTVEEGSIGGFGSHVAQFMALDGLLDGKLKWRPLVLPDRYIDHGSPADQLAEAGLTPSHIAATVFNVLGQAREALAIMTVPNA

SEQ ID NO: 6 - protein, 719 aa, Zea mays
MALSTFSVPRGFLGVPAQDSHFASAVELHVNKLLQARPINLKPRRRPACVSASLSSEREAEYYSQRPPTPLLDTINYPVHMKNLSVKELRQLADELRSDVIFHVSKTGGHLGSSLGVVELTVALHYVFNAPQDRILWDVGHQSYPHKILTGRRDKMPTMRQTNGLAGFTKRAESEYDSFGTGHSSTTISAALGMAVGRDLKGGKNNVVAVIGDGAMTAGQAYEAMNNAGYLDSDMIVILNDNKQVSLPTATLDGPVPPVGALSSALSKLQSSRPLRELREVAKGVTKQIGGSVHELAAKVDEYARGMISGPGSSLFEELGLYYIGPVDGHNIDDLITILNDVKSTKTTGPVLIHVVTEKGRGYPYAERAADKYHGVAKFDPATGKQFKSPAKTLSYTNYFAEALIAEAEQDSKIVAIHAAMGGGTGLNYFLRRFPSRCFDVGIAEQHAVTFAAGLACEGLKPFCAIYSSFLQRGYDQVVHDVDLQKLPVRFAMDRAGLVGADGPTHCGAFDVAYMACLPNMVVMAPSDEAELCHMVATAAAIDDRPSCFRYPRGNGVGVPLPPNYKGTPLEVGKGRILLEGDRVALLGYGSAVQYCLTAASLVQRHGLKVTVADARFCKPLDHALIRSLAKSHEVLITVEEGSIGGFGSHIAQFMALDGLLDGKLKWRPLVLPDRYIDHGSPADQLAEAGLTPSHIAASVFNILGQNREALAIMAVPNA

SEQ ID NO: 7 - protein, 342 aa, Arabidopsis thaliana
MADLKSTFLDVYSVLKSDLLQDPSFEFTHESRQWLERMLDYNVRGGKLNRGLSVVDSYKLLKQGQDLTEKETFLSCALGWCIEWLQAYFLVLDDIMDNSVTRRGQPCWFRKPKVGMIAINDGILLRNHIHRILKKHFREMPYYVDLVDLFNEVEFQTACGQMIDLITTFDGEKDLSKYSLQIHRRIVEYKTAYYSFYLPVACALLMAGENLENHTDVKTVLVDMGIYFQVQDDYLDCFADPETLGKIGTDIEDFKCSWLVVKALERCSEEQTKILYENYGKAEPSNVAKVKALYKELDLEGAFMEYEKESYEKLTKLIEAHQSKAIQAVLKSFLAKIYKRQK

SEQ ID NO: 8 - protein, 353 aa, Oryza sativa
MAAAVVANGASGDSSKAAFAEIYSRLKEEMLEDPAFEFTDESLQWIDRMLDYNVLGGKCNRGISVIDSFKMLKGTDVLNKEETFLACTLGWCIEWLQAYFLVLDDIMDNSQTRRGQPCWFRVPQVGLIAVNDGIILRNHISRILQRHFKGKLYYVDLIDLFNEVEFKTASGQLLDLITTHEGEKDLTKYNLTVHRRIVQYKTAYYSFYLPVACALLLSGENLDNFGDVKNILVEMGTYFQVQDDYLDCYGDPEFIGKIGTDIEDYKCSWLVVQALERADENQKHILFENYGKPDPECVAKVKDLYKELNLEAVFHEYERESYNKLIADIEAHPNKAVQNVLKSFLHKIYKRQK

SEQ ID NO: 9 - protein, 342 aa, Solanum lycopersicum
MADLKKKFLDVYSVLKSDLLEDTAFEFTDDSRKWVDKMLDYNVPGGKLNRGLSVIDSLSLLKDGKELTADEIFKASALGWCIEWLQAYFLVLDDIMDGSHTRRGQPCWYNLEKVGMIAINDGILLRNHITRILKKYFRPESYYVDLLDLFNEVEFQTASGQMIDLITTLVGEKDLSKYSLSIHRRIVQYKTAYYSFYLPVACALLMVGENLDKHVDVKKILIDMGIYFQVQDDYLDCFADPEVLGKIGTDIQDFKCSWLVVKALELCNEEQKKILFENYGKDNAACIAKIKALYNDLKLEEVFLEYEKTSYEKLTTSIAAHPSKAVQAVLLSFLGKIYKRQK

SEQ ID NO: 10 - protein, 533 aa, Zea mays
MDATAFHPSLWGDFFVKYKPPTAPKRGHMTERAELLKEEVRKTLKAAANQITNALDLIITLQRLGLDHHYENEISELLRFVYSSSDYDDKDLYVVSLRFYLLRKHGHCVSSDVFTSFKDEEGNFVVDDTKCLLSLYNAAYVRTHGEKVLDEAITFTRRQLEASLLDPLEPALADEVHLTLQTPLFRRLRILEAINYIPIYGKEAGRNEAILELAKLNFNLAQLIYCEELKEVTLWWKQLNVETNLSFIRDRIVECHFWMTGACCEPQYSLSRVIATKMTALITVLDDMMDTYSTTEEAMLLAEAIYRWEENAAELLPRYMKDFYLYLLKTIDSCGDELGPNRSFRTFYLKEMLKVLVRGSSQEIKWRNENYVPKTISEHLEHSGPTVGAFQVACSSFVGMGDSITKESFEWLLTYPELAKSLMNISRLLNDTASTKREQNAGQHVSTVQCYMLKHGTTMDEACEKIKELTEDSWKDMMELYLTPTEHPKLIAQTIVDFARTADYMYKETDGFTFSHTIKDMIAKLFVDPISLF

SEQ ID NO: 11 - protein, 533 aa, Zea mays; identical to SEQ ID NO: 10

SEQ ID NO: 12 - protein, 574 aa, Artemisia annua
MSTLPISSVSFSSSTSPLVVDDKVSTKPDVIRHTMNFNASIWGDQFLTYDEPEDLVMKKQLVEELKEEVKKELITIKGSNEPMQHVKLIELIDAVQRLGIAYHFEEEIEEALQHIHVTYGEQWVDKENLQSISLWFRLLRQQGFNVSSGVFKDFMDEKGKFKESLCNDAQGILALYEAAFMRVEDETILDNALEFTKVHLDIIAKDPSCDSSLRTQIHQALKQPLRRRLARIEALHYMPIYQQETSHDEVLLKLAKLDFSVLQSMHKKELSHICKWWKDLDLQNKLPYVRDRVVEGYFWILSIYYEPQHARTRMFLMKTCMWLVVLDDTFDNYGTYEELEIFTQAVERWSISCLDMLPEYMKLIYQELVNLHVEMEESLEKEGKTYQIHYVKEMAKELVRNYLVEARWLKEGYMPTLEEYMSVSMVTGTYGLMIARSYVGRGDIVTEDTFKWVSSYPPIIKASCVIVRLMDDIVSHKEEQERGHVASSIECYSKESGASEEEACEYISRKVEDAWKVINRESLRPTAVPFPLLMPAINLARMCEVLYSVNDGFTHAEGDMKSYMKSFFVHPMVV

SEQ ID NO: 13 - protein, 770 aa, Arabidopsis thaliana
MVAPALLPELWTEILVPICAVIGIAFSLFQWYVVSRVKLTSDLGASSSGGANNGKNGYGDYLIEEEEGVNDQSVVAKCAEIQTAISEGATSFLFTEYKYVGVFMIFFAAVIFVFLGSVEGFSTDNKPCTYDTTRTCKPALATAAFSTIAFVLGAVTSVLSGFLGMKIATYANARTTLEARKGVGKAFIVAFRSGAVMGFLLAASGLLVLYITINVFKIYYGDDWEGLFEAITGYGLGGSSMALFGRVGGGIYTKAADVGADLVGKIERNIPEDDPRNPAVIADNVGDNVGDIAGMGSDLFGSYAEASCAALVVASISSFGINHDFTAMCYPLLISSMGILVCLITTLFATDFFEIKLVKEIEPALKNQLIISTVIMTVGIAIVSWVGLPTSFTIFNFGTQKVVKNWQLFLCVCVGLWAGLIIGFVTEYYTSNAYSPVQDVADSCRTGAATNVIFGLALGYKSVIIPIFAIAISIFVSFSFAAMYGVAVAALGMLSTIATGLAIDAYGPISDNAGGIAEMAGMSHRIRERTDALDAAGNTTAAIGKGFAIGSAALVSLALFGAFVSRAGIHTVDVLTPKVIIGLLVGAMLPYWFSAMTMKSVGSAALKMVEEVRRQFNTIPGLMEGTAKPDYATCVKISTDASIKEMIPPGCLVMLTPLIVGFFFGVETLSGVLAGSLVSGVQIAISASNTGGAWDNAKKYIEAGVSEHAKSLGPKGSEPHKAAVIGDTIGDPLKDTSGPSLNILIKLMAVESLVFAPFFATHGGILFKYF

SEQ ID NO: 14 - protein, 782 aa, Oryza sativa
MNPSARISQVAMAAILPDLATQVLVPAAAVVGIAFAVVQWVLVSKVKMTAERRGGEGSPGAAAGKDGGAASEYLIEEEEGLNEHNVVEKCSEIQHAISEGATSFLFTEYKYVGLFMGIFAVLIFLFLGSVEGFSTKSQPCHYSKDRMCKPALANAIFSTVAFVLGAVTSLVSGFLGMKIATYANARTTLEARKGVGKAFITAFRSGAVMGFLLAASGLVVLYIAINLFGIYYGDDWEGLFEAITGYGLGGSSMALFGRVGGGIYTKAADVGADLVGKVERNIPEDDPRNPAVIADNVGDNVGDIAGMGSDLFGSYAESSCAALVVASISSFGINHEFTPMLYPLLISSVGIIACLITTLFATDFFEIKAVDEIEPALKKQLIISTVVMTVGIALVSWLGLPYSFTIFNFGAQKTVYNWQLFLCVAVGLWAGLIIGFVTEYYTSNAYSPVQDVADSCRTGAATNVIFGLALGYKSVIIPIFAIAFSIFLSFSLAAMYGVAVAALGMLSTIATGLAIDAYGPISDNAGGIAEMAGMSHRIRERTDALDAAGNTTAAIGKGFAIGSAALVSLALFGAFVSRAAISTVDVLTPKVFIGLIVGAMLPYWFSAMTMKSVGSAALKMVEEVRRQFNSIPGLMEGTTKPDYATCVKISTDASIKEMIPPGALVMLSPLIVGIFFGVETLSGLLAGALVSGVQIAISASNTGGAWDNAKKYIEAGASEHARTLGPKGSDCHKAAVIGDTIGDPLKDTSGPSLNILIKLMAVESLVFAPFFATHGGILFKWF

SEQ ID NO: 15 - protein, 762 aa, Triticum aestivum
MAILGELGTEILIPVCGVVGIVFAVAQWFIVSKVKVTPGAASAAGGGKNGYGDYLIEEEEGLNDHNVVVKCAEIQTAISEGATSFLFTMYQYVGMFMVVFAAVIFVFLGSIEGFSTKGQPCTYSTGTCKPALYTALFSTASFLLGAITSLVSGFLGMKIATYANARTTLEARKGVGKAFITAFRSGAVMGFLLSSSGLGVLYITINVFKMYYGDDWEGLFESITGYGLGGSSMALFGRVGGGIYTKAADVGADLVGKVERNIPEDGPRNPAVIADNVGDNVGDIAGMGSDLFGSYAESSCAALVVASISSFGINHDFTAMCYPLLVSSVGIIVCLLTTLFATDFFEIKAASEIEPALKKQLIIFTALMTIGVAVINWLALPAKFTIFNFGAQKDVSNWGLFFCVAVGLWAGLIIGFVTEYYTSNAYSPVQDVADSCRTGAATNVIFGLALGYKSVIIPIFAIAVSIYVSFSIAAMYGIAMAALGMLSTTATGLAIDAYGPISDNAGGIAEMAGMSHRIRERTDALDAAGNTTAAIGKGFAIGSAALVSLALFGAFVSRAGVKVVDVLSPKVFIGLIVGAMLPYWFSAMTRRVCESAALKMVEKVRRQFNTIPGLMKGTAKPDYATCVKISTDASIREMIPPGALVMLTPLIVGTLFGVETLSGVLAGALVSGVQIAISASNTGGAWDNAKKYIEAGNSEHARSLGPKGSDCHKAAVIGDTIGDPLKDTSGPSLNILIKLMAVESLVFAPFFATYGGVLFKYI

SEQ ID NO: 16 - DNA, 1851 bp, Artificial Sequence
  plant optimized sequence
ggatccgagctcatggatgttaggagaagaccaaccagcggcaagacgattcattccgttaagcccaagtcagtggaggacgagtcggcacagaagccctccgacgccttgccactcccgctgtaccttatcaacgctctctgcttcacagtgttcttttacgtggtctattttctcctgtcgcggtggagagaaaagattcgcacgtccactccccttcacgttgtggctttgagcgagatcgccgctattgtcgcgttcgttgcatcttttatctatcttttggggttctttggtatcgatttcgtccagtcattgattctccggccaccgacggacatgtgggccgttgacgatgacgaggaagagacagaagagggcattgtgctccgggaggatacgagaaagctgccgtgcgggcaagcccttgactgttcattgtcggcgcctcccctctctagggcagtcgtttccagccccaaggccatggacccaatcgtcctgcctagccccaagccaaaggttttcgacgaaattccgtttcctaccacaacgactatccccattctcggcgatgaggacgaagagatcattaagtcggtggtcgcgggcactatcccatcctacagcctcgaatccaagctgggggattgcaagagagcagcagcaatcaggagagaggcactccagaggattaccggaaagtctctgtcaggcctgccccttgaagggttcgactacgagagcatcctgggccagtgctgtgagatgccagtggggtatgtccaaatcccggtgggaattgccggccctctcctgcttgatggcaaggaatatagcgtgccaatggccaccacagagggttgcctggtcgcttctaccaaccgcggctgtaaggccatccatctttccggaggagctacgagcgtcttgctcagggatggcatgactagggccccagttgtgcggttcgggaccgcaaagagagctgcacagttgaagctctacctggaagaccctgccaactttgagaccctctcgacatccttcaataagtcttcaaggtttggtcgccttcaatccatcaagtgcgcaattgccggaaagaatctctatatgcgcttctgctgttctacaggggacgccatgggtatgaacatggtgtcaaagggcgttcagaacgtgctcaatttcctgcaaaatgattttccggatatggacgtgatcgggctgtctggtaacttctgctcagacaagaagcctgcagccgtcaattggattgaaggaaggggcaagagcgtcgtttgtgaggcgatcattaagggcgacgtggtcaagaaggtgctcaagactaacgtggaagcacttgtcgagttgaacatgctcaagaatctgaccggttcagctatggcgggagcactgggtggattcaacgcccacgcttcgaatatcgtcaccgccatctacattgctacaggccaggacccagcgcaaaacgtcgaatcgtccaattgcatcacaatgatggaggcagttaatgatggtcaggacctccatgtttcggtgacgatgccatccattgaggtcggcacggttggcgggggtactcagcttgcgagccaatctgcatgtttgaacctgcttggagtgaagggagcatccaaggagaccccaggtgcaaatagcagagtccttgcctctatcgttgctggatcagtgttggctgcggagctttcattgatgtcggccattgcagccggccagctggttaactcccacatgaagtacaacagggctaataaggaggctgcggtcagcaagcctagctcttgaggtacctctagaaagctt

SEQ ID NO: 17 - DNA, 1605 bp, Artificial Sequence
  plant optimized sequence
ggatccgagctcatggctgccgatcaactggtgaagaccgaggttactaagaagtcgtttactgcccctgtccaaaaggcgtccactcccgtgctgaccaacaagaccgttatctcgggttccaaggtgaagtccctctccagcgcccagtcttcatcgtccggaccatcctcctcctccgaggaagacgattcgcgggacatcgagtccctggataagaagattagacctctcgaggaactggaagccctcctgtccagcggcaacacaaagcaactcaagaataaggaggttgccgctctcgtgatccacggcaagctccccttgtacgctcttgaaaagaagttgggagacaccacaagggcggttgcagtgaggcgcaaggcgctttcgattttggccgaggctccggtgctcgcatcagataggctgccttataagaactacgactatgatcgcgtgttcggcgcctgctgtgagaatgtcatcgggtacatgccacttccggtcggtgttatcggacccctcgtgatcgacggcacatcttatcatatcccaatggcgacgactgagggttgcctcgtcgcaagcgcaatgagaggctgtaaggccattaacgctggcgggggtgcaaccacagtgctgactaaggacggtatgaccaggggaccagtggtccgcttccctacgcttaagcgctctggcgcctgcaagatttggctcgattcagaggaagggcagaacgcgattaagaaggcattcaatagcacatctaggtttgcgcgcctccagcacatccaaacgtgtctggcaggtgaccttttgttcatgcggtttagaacaactaccggcgatgctatggggatgaatatgatttcaaagggcgttgagtactcgctcaagcaaatggtggaggaatatggttgggaggacatggaagttgtgtcagtgtcgggaaactactgcactgataagcccgcggcaatcaattggattgagggaagggggaagtccgtcgttgcagaagctaccatcccaggcgacgtggtcagaaaggtcctgaagtctgatgtctcagccctcgttgagctgaacattgctaagaatcttgtcggtagcgcgatggcaggatctgttggaggcttcaacgcccatgccgctaatctggtgacagccgtctttctcgctctgggccaggaccctgctcaaaacgtggagtcttcaaattgcatcacgctcatgaaggaagtcgacggggatctgcggatttccgtcagcatgccgagcatcgaggttggcacaattgggggtggaacggttcttgaacctcagggggcgatgttggatctcctgggcgtcagaggaccacacgcaacagctccaggcacgaacgcgcggcaactcgcaagaatcgtggcatgcgcagtcctggcaggagagctttccttgtgtgcggcacttgccgctgggcatttggtgcagagccacatgactcataacaggaagcctgccgagcccactaagccaaacaatcttgacgctaccgatatcaatcgcttgaaggacggctccgtcacctgcattaagagctaaggtaccaagctt

SEQ ID NO: 18 - DNA, 2193 bp, Artificial Sequence
  plant optimized sequence
ggatccgagctcatggcgttgactacattttcgatttcacgggggggtttcgttggagccctgccgcaagaaggacactttgcacctgccgctgctgagctttcgttgcacaagctgcagtcccggcctcataaggcaaggagacggtccagctcttcaatcagcgcatctctctcaacggagcgggaagccgctgagtaccactctcaaagaccaccgacgcctctcctggacactgtgaactatcccatccatatgaagaatctcagcctgaaggagcttcagcaattggcggacgaactgcgctccgatgtcattttccacgttagcaagacgggcgggcatcttggatcgtccttgggagtggtcgagctgacggtggcactgcactacgtctttaacactccgcaggacaagatcctctgggatgtcggacaccaatcctatcctcataagattctgactggcagaagggacaagatgcccacgatgaggcagactaatggtctctccggattcaccaagcgctcggagtccgaatacgattcgtttggaacaggccatagctctaccacaatctccgcagcattgggaatggcagtgggtagggacctcaagggtggaaagaacaatgttgtggcagtcattggggatggtgcgatgaccgcaggacaggcctacgaggctatgaacaatgccggctatctggacagcgatatgatcgttattcttaacgacaataagcaagtgtctctgcctaccgcaacacttgatggaccagcacctccagtgggtgcgctgtcatcggcactcagcaagctgcagtccagccgccctcttcgggagttgagagaagtggccaagggcgtcaccaagcaaatcggcgggtccgttcacgagctggccgctaaggtggacgaatacgctcgggggatgattagcggatctggctcaacactcttcgaggaacttggcttgtactatatcggacccgtggatggccataacattgacgatcttatcacgattttgagagaggtgaagtccactaagacgactggcccagtcctcatccacgtcgttacggagaaggggaggggttacccgtatgcggaacgcgcggcagacaagtaccatggggtcgcgaagttcgatccagcaactggcaagcagtttaagagcccggcaaagaccttgtcttacacaaactatttcgccgaggctcttatcgcggaggcagaacaagacaatagggtggtcgctattcacgcagctatgggtggaggcaccggcctcaactatttcctgcgccggtttccaaatcgctgcttcgatgtcggcatcgccgagcagcatgctgttacatttgcggcaggattggcctgcgaaggcctcaagccgttctgtgctatctactcttcatttctgcagaggggctatgaccaagttgtgcacgacgtcgatctccagaagctgcctgttcggttcgcgatggacagagcaggactcgtcggagctgatggtccaacccattgcggagcctttgacgttacatacatggcttgtcttccaaacatggtcgttatggccccgtccgatgaggctgaactctgccacatggtggcaaccgcagctgcaatcgacgatagaccaagctgtttccgctacccacgcggaaacggcattggggtccctctgccaccgaattataagggcgttccccttgaggtcggcaagggacgggtgcttttggagggtgaaagagtcgcgctcctgggctacgggtctgcagttcagtattgcctggcagccgcttcacttgtggagagacacggactgaaggtgacggtcgccgacgctagattctgtaagccacttgatcaaactttgatcagaaggctcgcctcgtcccacgaggtccttttgaccgttgaggaaggatcaattgggggtttcggctcgcatgtggcccagtttatggctttggacgggctcctggatggcaagctcaagtggaggcctctcgtcctgcccgaccgctacatcgatcacgggtcaccagcagaccagttggcagaggcaggtctcaccccgtcgcatatcgcggcaacagttttcaacgtgctgggacaagcaagagaagcccttgctattatgacagtgccgaatgcttgaggtacctctagaaagctt

SEQ ID NO: 19 - DNA, 2193 bp, Artificial Sequence
  plant optimized sequence
ggatccgagctcatggccctctctgcgtgttcgttccctgctcatgttgacaaggcgactatcagcgacctccaaaagtatggttatgtgcccagccgcagcctctggagaacggacctcctggcccagagcttgggaaggctcaaccaggctaagtctaagaagggacctggaggaatctgcgcttccctgagcgagagaggcgaataccactcacagaggccaccgactcctcttttggacaccacaaactatcccatccatatgaagaatcttagcattaaggagctgaagcaacttgccgacgaattgcgctcggatgtgatcttcaacgtctcccggacgggtggacacttgggctcctccctcggagtggtcgagctgactgttgcgcttcattacgtgttctcagcacctcgggacaagatcctttgggatgtggggcaccagtcctacccccataagatcctcaccggtaggcgcgagaagatgtatacgattcgccaaactaatggcctctctgggttcaccaagcggtctgagtcagaatacgactgctttggaacaggccactcttcaacgactatctccgcaggactcggtatggcagtgggaagggacctgaagggcaagaagaacaacgttgtggcagtcattggagatggcgcgatgacagcagggcaggcctacgaggctatgaacaatgccggttatcttgactcagatatgatcgttatcttgaacgacaataagcaagtgtcgctccctaccgccacactggatggaccaatccctccagtgggcgcgctgtcgtccgcattgtcgagactccagtccaacaggcctctgcgcgagcttcgggaagttgcaaagggcgtgaccaagcaaatcggaggaccaatgcacgagtgggcagctaaggtggacgaatacgcccgcggcatgatttcggggtccggtagcacactcttcgaggaacttggcttgtactatatcgggcctgtcgatggtcataatattgacgatttgatcgctattctcaaggaggtgaagtccacgaagaccacaggcccagtcctgatccacgtcgttactgagaagggacgcggctacccgtatgcggaaaaggcggcagacaagtaccatggcgtcaccaagttcgatcccgcgacaggaaagcagtttaagggctcagcaatcacgcaatcgtacacgacttatttcgccgaggctctcattgcggaggcagaagtcgacaaggatatcgttgccattcacgcagctatgggtggaggcacggggctcaacctgttccttcggagatttccaactcgctgcttcgacgtcggcatcgccgagcagcatgctgttacctttgcggcagggcttgcctgcgaaggtttgaagccgttctgtgctatctacagctcttttatgcagcgggcgtatgatcaagtggtccacgacgtggatttgcagaagctcccagtccgcttcgcgatggacagagcaggtctcgtgggagcagatggaccaacccattgcggagcattcgacgtcaccttcatggcttgtctgccaaatatggttgtgatggccccgagcgatgaggctgaacttttccacatggtggcaaccgcagctgcaatcgacgatagaccatcttgttttagatacccgagggggaacggtgtcggagttcagctgccaccggggaataagggtattccgctcgaggtcggcaagggacgcatcctgattgagggcgaacgggttgcgctcctgggttatggaaccgcagtgcagtcctgcctcgcagcagctagcctggtcgagcctcacggccttttgatcaccgttgccgacgctagattctgtaagcccctggatcacacacttattaggagcttggccaagtctcatgaggtcctcatcacagttgaggaagggtctattgggggtttcggttcacacgtggcccacttcctcgctctcgacggactcctggatggcaagctgaagtggagacctctggttcttcccgacaggtacatcgatcacggatctccatcagtccagcttattgaggctggattgacgccaagccatgtggcagcaactgtcctgaacatccttggcaataagagggaagcgctgcaaattatgtcatcgtgaggtacctctagaaagctt

SEQ ID NO: 20 - DNA, 2175 bp, Artificial Sequence
  plant optimized sequence
ggatccgagctcatggcgttgactacattttcgatttcacgggggggtttcgttggagccctgccgcaagaaggacactttgcacctgccgctgctgagctttcgttgcacaagctgcagtcccggcctcataaggcaaggagacggtccagctcttcaatcagcgcgtctctgtcagagagaggcgaataccacagccagaggccaccgacacctcttttggacacgactaactatcccatccatatgaagaatctttctattaaggagctgaagcaacttgccgacgaactccgctccgatgtgatcttcaacgtcagccggaccggaggacacttggggtccagcctcggtgtggtcgagctgacagttgcgcttcattacgtgttcagcgcacctcgcgacaagatcctgtgggatgtcggacaccagtcttacccccataagatccttacgggcaggcgcgagaagatgtataccattagacaaacaaatggtctctccggattcacgaagaggtcggagtccgaatacgactgctttgggactggtcactcttcaaccacaatctccgcaggactcggaatggcagtgggaagggacctgaagggcaagaagaacaatgttgtggcagtcattggggatggtgccatgaccgctggacaggcgtacgaggccatgaacaacgccggctatcttgactcggatatgatcgttattttgaacgacaataagcaagtgtccctccctacggctactctggatggaccaatccctccagtgggtgccctgtcgtccgctttgtcccgcctccagagcaaccggccactgagagagcttcgcgaagttgcaaagggcgtgaccaagcaaatcggtggaccgatgcacgagtgggccgctaaggtggacgaatacgcccgggggatgattagcggatctggctcaacactcttcgaggaacttggtttgtactatatcggacctgtcgatggccataatattgacgatttgatcgctattctcaaggaggtgaagtccaccaagacgactggcccagtcctgatccacgtcgttacagagaaggggcgcggttacccgtatgcggaaaaggcggcagacaagtaccatggcgtcacgaagttcgatccggcgactgggaagcagtttaagggttcggcaatcacccaatcctacaccacatatttcgccgaggctctcattgcggaggcagaagtcgacaaggatatcgttgccattcacgcagctatgggaggaggcaccggcctcaacctgttccttcggagatttcctacaagatgcttcgacgtcggcatcgcggagcagcatgcagttacatttgcggcaggacttgcctgcgaaggcttgaagcccttctgtgctatctacagctcttttatgcagagggcgtatgatcaagtggtccacgacgtggatttgcagaagctcccagtccgcttcgccatggacagagctggactcgtgggagcagatggtccaacgcattgcggagccttcgacgtcacttttatggcttgtctcccaaacatggttgtgatggccccgtcagatgaggctgaactgttccacatggtggctaccgcagctgcaatcgacgatagaccatcctgttttcgctacccgagaggaaacggcgtcggagttcagctgccaccgggaaataagggcattccgctcgaggtcggcaagggacgcatcctgattgagggcgaacgggttgcgctcctgggctatgggacggcagtgcagagctgcctcgcagcagcttctctggtcgagcctcatggccttttgatcacggttgccgacgctcgcttctgtaagcccctggatcacactcttattcggtctttggccaagtcacatgaggtcctcatcactgttgaggaaggatcaattggaggcttcggctcgcacgtggcgcacttcctcgcactcgacgggctcctggatggcaagctcaagtggagacctctggttcttcccgacaggtacatcgatcacgggtcgccatccgtgcagcttattgaggctggtttgaccccgagccatgtggcggcaacagtcctgaacatccttggcaataagagggaagcgctgcaaattatgtcatcgtgaggtacctctagaaagctt

SEQ ID NO: 21 - DNA, 1227 bp, Artificial Sequence
  plant optimized sequence
ggatccgagctcatggcaccgacagttatggcatcatccgctacagccgttgctcctttccaggggttgaagtccaccgctactcttcccgttgcgaggaggtccaccacctccttcgcgaaggtgtcaaacggcgggaggatcaggtgcatggcatcggagaaggaaattaggcgcgagcgcttcctgaacgtctttcctaagctggttgaggaacttaatgcctcgctcctggcttacggcatgcccaaggaggcctgtgactggtacgctcactccctcaactataatacgccaggtggaaagttgaacagggggctcagcgtggtcgatacgtacgccatcctgtctaataagactgtcgagcagcttggtcaagaggaatatgaaaaggttgctatcttgggatggtgcattgagcttttgcaggcgtacttcctggtcgcagacgatatgatggacaagtccatcacccggagaggccaaccatgttggtataaggttccggaagtgggggaaatcgcgattaacgacgcattcatgctggaggccgctatctacaagctcctgaagtcacactttcgcaacgagaagtactatatcgacattacggagctgttccatgaagttacgtttcagactgagctgggccaactgatggatcttatcactgcgcccgaagacaaggtggatctgtctaagttctcacttaagaagcactccttcattgtcacctttaagacagcctactatagcttttacctgcctgtggcgcttgcaatgtatgtcgccggcatcacagacgagaaggatcttaagcaggctcgggacgtgttgatcccgctcggcgagtacttccagattcaagacgattatctcgattgctttggaacccctgagcagatcggcaagattgggacagacatccaagataacaagtgttcttgggttattaataaggcccttgagttggcctcagctgaacagagaaagaccctggacgagaactacggcaagaaggatagcgtggcggaagcaaagtgcaagaagattttcaacgacttgaagattgagcagctctaccatgaatatgaggaatctatcgccaaggatctcaaggctaagatttcgcaagtcgacgagtcccggggcttcaaggcggatgttttgacagcatttctcaataaggtgtacaagagatccaagtgaggtacctctagaaagctt

SEQ ID NO: 22 - DNA, 1059 bp, Artificial Sequence
  plant optimized sequence
ggatccgagctcatggctgatctgaagtcgacgtttttgaaggtgtattccgttctgaagcaggagttgctggaggaccccgcatttgagtggacccctgactccaggcagtgggtcgagcgcatgctcgattacaacgttcctggcgggaagctcaatcggggcctgtctgtgattgactcatataagctcctgaaggaggggcaagaacttaccgaggaagagattttcctcgcgtccgcattgggttggtgcattgagtggttgcaggcctactttctcgtcctggacgatatcatggactccagccacacaaggcgcggccaaccttgttggttcagggtgcccaaggtcggactgatcgcagctaacgatgggattcttttgcggaatcacatcccccgcatcctcaagaagcattttcgcggcaaggcttactatgttgacctcctggatttgttcaacgaagtggagtttcagaccgcgtctggtcaaatgatcgacctcattaccacactggaaggagagaaggatctctcgaagtacaccctttccttgcaccggagaatcgtccagtacaagacagcatactatagcttctatctgccagttgcctgcgctcttttgattgccggcgagaacctcgacaatcatatcgtggtcaaggatattctggtgcagatgggtatctacttccaggtccaagacgattatctcgactgttttggagatccggagacgatcggcaagatcggaactgacatcgaagatttcaagtgctcctggctcgttgtgaaggcactcgagctgtgtaacgaggagcagaagaaggtgctgtacgaacactatggcaaggccgacccagcaagcgtcgccaaggtcaaggttctttacaacgagcttaagttgcaaggggttttcacggaatacgagaacgagtcatataagaagctggtcactagcatcgaggctcatccatctaagccggttcaggctgtgcttaagtcgtttttggcgaagatatacaagaggcaaaagtgaggtacctctagaaagctt

SEQ ID NO: 23 - DNA, 1197 bp, Artificial Sequence
  plant optimized sequence
ggatccgagctcatggcaccaaccgtcatggcatcgtccgcaaccgccgtcgcacctttccagggtctgaagtcaacagcaacactcccagtcgcaagaaggtctaccacatcattcgcaaaggtgtccaacggcgggaggatcaggtgcatggccgaccttaagtccacgttcttgaaggtgtacagcgtcctcaagcaggagctgctcgaggacccagcttttgagtggactcccgattcacggcaatgggtggaaagaatgctggactacaacgtcccaggtggcaagctcaatcgcggtttgtccgtgatcgattcctacaagctcttgaaggagggacaggaacttaccgaggaagagattttcctcgcgtccgcactgggctggtgcattgagtggttgcaggcctactttcttgtcttggacgatatcatggactccagccacacaaggcgcgggcaaccatgttggttccgggttccgaaagtgggtctcatcgccgctaacgatggcatcctcctgaggaatcacatcccgcgcattcttaagaagcattttagaggcaaggcatactatgtcgaccttttggatttgttcaacgaagttgagtttcagacggccagcggccaaatgatcgaccttattacgactttggaaggggagaaggatcttagcaagtacacgctctctctgcaccggagaatcgtgcagtacaagactgcttactattctttctatctgcctgtcgcctgcgctctcctgattgcgggcgagaacctcgacaatcatatcgtggtcaaggatattctggttcagatgggcatctacttccaggtgcaagacgattatctggactgttttggcgacccagagaccatcggcaagattgggacagacatcgaagatttcaagtgctcgtggctcgttgtgaaggctcttgagttgtgtaacgaggagcagaagaaggttctgtacgagcactatggcaaggcggacccagcatccgtcgccaaggtcaaggttctctacaacgagctgaagctgcaaggagtgttcaccgaatacgagaacgagtcttataagaagctggtcacatcaatcgaggcgcatccatcgaagccggtccaggctgttctcaagtcatttctggcgaagatatacaagcggcaaaagtgaggtacctctagaaagctt

SEQ ID NO: 24 - DNA, 1083 bp, Artificial Sequence
  plant optimized sequence
ggatccgagctcatggcgtcagagaaggagattagaagggagaggtttttgaatgttttccccaagctggttgaagagttgaatgcgtcactgctggcatacggtatgcctaaggaggcgtgcgactggtacgcacactccctgaactataatacccccggcgggaagttgaaccggggactctcggtggtcgatacctacgccatcctgtccaataagacagttgagcagcttggccaagaggaatatgaaaaggtggctatcttggggtggtgcattgagctgctgcaggcctacttcctcgttgctgacgatatgatggacaagtctatcacaaggcgcggtcaaccatgttggtataaggttccggaagtgggagaaatcgccattaacgacgctttcatgctggaggccgctatctacaagctcttgaagagccactttcgcaacgagaagtactatatcgacattaccgagctgttccatgaagtcacctttcagacagagcttggtcaattgatggatctcatcacagcccctgaagacaaggtcgatctgtccaagttcagccttaagaagcacagcttcattgttacgtttaagactgcgtactattctttctacctgccggtcgcgcttgcaatgtatgttgcgggcatcacggacgagaaggatctgaagcaggcaagggacgtgctgatcccacttggcgagtacttccagattcaagacgattatcttgattgctttgggacgccggagcagatcggcaagatcggaactgacatccaagataacaagtgttcatgggtcatcaacaaggccctcgagctggcatcggctgaacagcgcaagacgctggacgagaactacggcaagaaggattccgtcgcggaagcaaagtgcaagaagattttcaacgacttgaagattgagcagctctaccatgaatatgaggaaagcatcgcgaaggatctcaaggcaaagatttctcaagtcgacgagtcacgggggttcaaggccgatgtgttgactgcttttctcaacaaggtctacaagagatccaagtaaggtaccaagctt

SEQ ID NO: 25 - DNA, 1893 bp, Artificial Sequence
  plant optimized sequence
ggatccgagctcatggcccctacggtcatggcgtcctcagcgactgcggttgcaccctttcaaggtctcaagagcacggcgacactccctgtggcacggagatcgaccacatccttcgccaaggtttccaacggcgggagaatcaggtgcatggacacgctgccaatttccagcgtctcattttcttcatcgacttcgcctcttgtggtcgacgataaggtttcgacgaagcccgacgtgatcaggcacactatgaacttcaatgcttcaatttggggcgatcagtttctgacctacgacgagccagaggacctcgtgatgaagaagcaactcgttgaggaactgaaggaggaagtgaagaaggagctgatcacaattaagggtagcaatgagccgatgcagcacgtgaagctcatcgagttgattgacgcggtccaacgcttgggaatcgcataccatttcgaggaagagatcgaagaggcccttcagcacattcatgtcacctacggcgagcagtgggttgataaggaaaacttgcaatcaatttcgctctggttccgcctcctgcggcagcaaggttttaatgtgtccagcggagtcttcaaggactttatggatgagaagggcaagttcaaggaatctctctgcaacgacgcgcagggaatccttgcattgtacgaggccgctttcatgcgggtggaggacgaaaccattcttgataatgcgttggagtttacaaaggtccacttggatatcattgcaaaggacccgtcatgtgattcttcactcagaacccagatccatcaagccctcaagcagccactgaggagaagacttgcaaggatcgaggcactgcactacatgccgatctaccagcaagagacatcccatgacgaagttcttttgaagctcgctaagctggatttctcggtgttgcagtccatgcacaagaaggagctgagccatatctgcaagtggtggaaggacctcgatctgcaaaacaagctgccttacgtgcgcgaccgggttgtggagggctatttctggattctctccatctactatgagccccagcacgcgagaaccaggatgtttctgatgaagacatgcatgtggcttgtcgttttggacgatacgttcgacaattacggtacttatgaagagctggagattttcacccaagcagtggaacgctggtccattagctgtctcgatatgctgcctgagtacatgaagctcatctatcaggagcttgttaacttgcacgtggagatggaggagagcctggagaaggaagggaagacgtaccaaattcattatgtcaaggagatggccaaggaactggtgagaaattaccttgtcgaggctaggtggctgaaggaaggctacatgcccacccttgaagagtatatgtctgtctcaatggttacgggcacttacgggctcatgatcgcgcgctcttatgtgggtcggggagacattgtcaccgaggatacattcaagtgggtctcgtcctacccaccgatcattaaggcgtcctgcgttatcgtgcgcctgatggacgatattgtcagccacaaggaagagcaggagcggggccatgttgcaagctctatcgagtgctacagcaaggaatctggggcctccgaagaggaggcctgcgagtatatctctcgcaaggttgaagacgcctggaaggtcatcaacagagagtcactgaggccaacggctgtgcctttccccctcctgatgccggccatcaacttggctcggatgtgtgaggtcctctacagcgttaatgacggcttcactcacgccgagggggatatgaagagctatatgaagtctttctttgtccatcctatggtggtctgaggtacctctagaaagctt

SEQ ID NO: 26 - DNA, 1749 bp, Artificial Sequence
  plant optimized sequence
ggatccgagctcatggataccctgcctatttcgtccgtctcgttctcctcttctacgtcgccactggtcgtcgatgataaggtgtctacaaagcctgatgtgatccgccacacgatgaacttcaatgcctctatctggggcgaccagtttctgacttacgacgagcctgaggacctcgtgatgaagaagcaactcgtcgaggaactgaaggaagaagtcaagaaggagctgatcacgattaagggctcaaacgagcccatgcagcacgtgaagctcatcgagttgattgacgcggtgcaaaggctggggatcgcataccatttcgaggaagagatcgaagaggctcttcagcacattcatgtgacatacggcgagcagtgggtcgataaggaaaacttgcaatcaatttcgctctggttcagactcctgaggcagcaaggctttaatgtctccagcggggttttcaaggactttatggatgagaagggcaagttcaaggaatcgctctgcaacgacgcgcagggcatcctcgcattgtacgaggccgctttcatgcgcgttgaggacgaaaccattcttgataatgcgttggagtttacaaaggtccacttggatatcattgcaaaggacccttcttgtgattcttcactccgcacgcagatccatcaagccctcaagcagcctctgaggagaagacttgcaagaatcgaggcactgcactacatgcccatctaccagcaagagacttcccatgacgaagtccttttgaagctcgctaagctggatttctctgttttgcagtcaatgcacaagaaggagctgagccatatctgcaagtggtggaaggacctcgatctgcaaaacaagttgccatacgtgagagacagggtggtcgaggggtatttctggattctctccatctactatgagccgcagcacgcgcgcacgcggatgtttctgatgaagacttgcatgtggcttgttgtgttggacgataccttcgacaattacggcacatatgaagagctggagattttcacccaagcagtggaaaggtggtccattagctgtctcgatatgctgccagagtacatgaagctcatctatcaggagcttgtgaacttgcacgtcgagatggaggagagcctggagaaggaaggaaagacctaccaaattcattatgtcaaggagatggccaaggaactggtccgcaattaccttgttgaggctcggtggctgaaggaaggctacatgccgacacttgaagagtatatgtctgtttcaatggtgaccggtacatacggactcatgatcgccagatcctatgttggcaggggggacattgtgacggaggatactttcaagtgggtgtcgtcctacccaccgatcattaaggcgagctgcgtgatcgtcagactgatggacgatattgtgtctcacaaggaagagcaggagaggggtcatgtcgcaagctctatcgagtgctactcgaaggaatccggagccagcgaagaggaggcctgcgagtatatctcaagaaaggtcgaagatgcctggaaggttattaatagagagtcgctgagaccaaccgctgtgcctttcccactcctgatgccggccatcaacttggctcggatgtgtgaggttctctacagcgtgaatgacggttttacacacgccgagggagatatgaagtcgtatatgaagtccttctttgtccatccaatggtcgtttaaggtaccaagctt

SEQ ID NO: 27 - DNA, 2373 bp, Artificial Sequence
  plant optimized sequence
ggatccgagctcatgaatccttccgcaagaatttcgcaagtggcaatggcagcaatcctccccgatctggctacgcaggtgttggttcccgccgcagcggtggtcggcatcgctttcgcggttgtgcagtgggtgctggtctctaaggtcaagatgacggcagagaggagaggaggagaaggatctcctggagcagctgcaggcaaggacggtggagcagcctcagagtaccttatcgaggaagaggaagggttgaacgaacacaatgtcgttgagaagtgctccgaaatccagcatgcgatttcggagggcgcaacctccttcctctttacagaatacaagtatgtggggctttttatgggtatcttcgccgtcttgatcttcctcttcctcggatctgttgagggcttctctaccaagtcacaaccttgccactactcaaaggataggatgtgtaagcccgcacttgccaacgctatctttagcaccgttgccttcgtgttgggcgctgtgacatcgcttgtctccgggttcttgggtatgaagatcgccacctatgcgaatgcaagaaccacactggaggctaggaagggagtcggcaaggcgtttattacagcattcagaagcggggccgtgatgggtttcctcctggctgcgtctggcctcgtggtcctgtacatcgctattaacctctttggaatctactatggcgacgattgggagggcctgttcgaagccattacgggatacggtctcggagggtccagcatggctctgttcggtagggttggtggaggcatctatactaaggcagccgacgtgggtgctgatctcgtcggaaaggttgagcgcaacattccagaagacgatcctcggaatcccgccgtgatcgcagacaacgttggggataatgtgggtgacattgcgggaatgggcagcgaccttttcggctcttacgcggagtcttcatgcgctgcgttggttgtggcatccatctcgtcctttggcattaatcatgagttcaccccaatgctgtatccgcttttgattagctctgtcgggatcattgcgtgtcttatcacgactttgttcgcaactgacttctttgagatcaaggccgtggatgagattgaacctgctctcaagaagcagctgatcattagcacggtcgttatgactgtgggcatcgcgctcgtctcttggctcgggctgccctactcattcacgattttcaactttggcgcccagaagactgtctataattggcaactcttcctctgcgttgcggtgggactttgggcaggcttgatcattgggttcgtgaccgagtactatacatccaacgcctacagcccagtgcaagacgtcgctgatagctgtcgcacgggcgcagccactaatgtcatctttggtctcgccctgggatataagtcagttatcattccgatcttcgccattgctttctcgatctttctctcattctcgctggctgcgatgtacggcgtcgcggttgcagcccttgggatgttgtccaccatcgcaacaggtctggccattgacgcttatggaccaatctcggataacgccgggggtattgcggagatggccggtatgagccacaggatcagggaacggaccgacgcgcttgatgctgcgggaaataccacagcagccattgggaagggtttcgcaatcggttcagctgcgctggtgtcgcttgccttgtttggagctttcgtctccagagcagcaatcagcacggtggacgtcctcactccaaaggtttttatcggcctcattgtgggggcgatgctgccgtactggttctccgcaatgaccatgaagagcgtcggctctgctgcgctcaagatggttgaggaagtgcggagacagttcaacagcatcccaggtctgatggagggaacgactaagccggactacgccacctgcgtcaagatttctacagatgcttcaatcaaggagatgattccaccaggcgccctcgtgatgctgtccccacttatcgtcggcattttctttggggttgagacactctcgggtctcctggcaggagcactggtctccggcgttcaaatcgccatttccgctagcaacaccggaggcgcgtgggacaatgcaaagaagtacatcgaggcaggagcttccgaacacgcacgcacactgggacctaagggcagcgattgtcataaggcagccgtgatcggcgatacgattggggaccctctcaaggatacttcaggcccctcgttgaacatcctcattaagctgatggctgtcgagtccctggttttcgcccccttctttgctacccatgggggtatcctttttaagtggttctaaggtaccaagctt

SEQ ID NO: 28 - DNA, 1851 bp, Artificial Sequence; identical to SEQ ID NO: 16
  plant codon optimized

SEQ ID NO: 29 - DNA, 1059 bp, Artificial Sequence
  plant codon optimized
atggcgtcagagaaggagattagaagggagaggtttttgaatgttttccccaagctggttgaagagttgaatgcgtcactgctggcatacggtatgcctaaggaggcgtgcgactggtacgcacactccctgaactataatacccccggcgggaagttgaaccggggactctcggtggtcgatacctacgccatcctgtccaataagacagttgagcagcttggccaagaggaatatgaaaaggtggctatcttggggtggtgcattgagctgctgcaggcctacttcctcgttgctgacgatatgatggacaagtctatcacaaggcgcggtcaaccatgttggtataaggttccggaagtgggagaaatcgccattaacgacgctttcatgctggaggccgctatctacaagctcttgaagagccactttcgcaacgagaagtactatatcgacattaccgagctgttccatgaagtcacctttcagacagagcttggtcaattgatggatctcatcacagcccctgaagacaaggtcgatctgtccaagttcagccttaagaagcacagcttcattgttacgtttaagactgcgtactattctttctacctgccggtcgcgcttgcaatgtatgttgcgggcatcacggacgagaaggatctgaagcaggcaagggacgtgctgatcccacttggcgagtacttccagattcaagacgattatcttgattgctttgggacgccggagcagatcggcaagatcggaactgacatccaagataacaagtgttcatgggtcatcaacaaggccctcgagctggcatcggctgaacagcgcaagacgctggacgagaactacggcaagaaggattccgtcgcggaagcaaagtgcaagaagattttcaacgacttgaagattgagcagctctaccatgaatatgaggaaagcatcgcgaaggatctcaaggcaaagatttctcaagtcgacgagtcacgggggttcaaggccgatgtgttgactgcttttctcaacaaggtctacaagagatccaagtaa